(19) United States
(12) Patent Application Publication
(10) Pub. No.: US 2002/0192675 A1
(43) Pub. Date: Dec. 19, 2002

(54) Methods of identifying regulator molecules

(75) Inventors: Maurice Zauderer, Pittsford, NY (US); Ernest S. Smith, Ontario, NY (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: The University of Rochester, Rochester, NY

(21) Appl. No.: 10/061,395

(22) Filed: Feb. 4, 2002

Related U.S. Application Data
(60) Provisional application No. 60/265,589, filed on Feb. 2, 2001.
(60) Provisional application No. 60/265,880, filed on Feb. 5, 2001.
(60) Provisional application No. 60/271,423, filed on Feb. 27, 2001.

(51) Int. Cl. C12Q 1/68; G01N 33/53; G01N 33/567; C12N 15/87

(52) U.S. Cl. 435/6; 435/7.1; 435/7.2; 435/455

(57) ABSTRACT

The invention provides methods and compositions for identifying, i.e., selecting and/or screening for regulator molecules such as polypeptides and/or U1 SnRNAs which irectly or indirectly influence, e.g., induce or suppress, the transcriptional activation of a target transcriptional regulatory region in a eukaryotic host cell. Also provided are regulator molecules identified by such methods, and methods of isolating polynucleotides encoding regulator molecules identified by these methods.

### CROSS-REFERENCE TO RELATED APPLICATIONS

The present application claims benefit of the filing dates of the following applications: U.S. Provisional Application No. 60/265,589, filed Feb. 2, 2001, U.S. Provisional Application No. 60/265,880, filed Feb. 5, 2001, and U.S. Provisional Application No. 60/271,423, filed Feb. 27, 2001; each of which are incorporated herein by reference in their entireties.

### BACKGROUND OF THE INVENTION

1. Field of the Invention

The present invention relates to a high efficiency method of expressing regulator molecules in eukaryotic host cells, a method of producing libraries of regulator molecules for expression in eukaryotic cells, methods of identifying regulator molecules which directly or indirectly influence the transcriptional activation of a target transcriptional regulatory region, regulator molecules identified by such methods, and methods of isolating polynucleotides encoding regulator molecules identified by any of these methods. Desired regulator molecules are selected from and/or screened for from libraries of polynucleotides containing randomized sequences.

2. Related Art

Transcriptional regulatory pathways, also referred to as signaling pathways, in cells often begin with an effector stimulus that leads, often through complex signal transduction pathways, to a change in cellular physiology resulting in an observable phenotypic modification. Despite the key role transcriptional regulatory pathways play in cellular differentiation, cell death, immune cell activation, intracellular interactions, cancer development, and disease pathogenesis in general, in most cases, little is understood about a given regulatory pathway other than the initial stimulus and the ultimate cellular response.

Historically, signal transduction has been analyzed by biochemistry or genetics. The biochemical approach dissects a pathway in a “stepping-stone” fashion: find a molecule that acts at, or is involved in, one end of the pathway, isolate assayable quantities and then try to determine the next molecule in the pathway, either upstream or downstream of the isolated one. The genetic approach is classically a “shot in the dark”: induce or derive mutants in a signaling pathway and map the locus by genetic crosses or complement the mutation with a cDNA library. Limitations of biochemical approaches include a reliance on a significant amount of pre-existing knowledge about the constituents under study and the need to carry such studies out in vitro, post-mortem. Limitations of purely genetic approaches include the need to first derive and then characterize the pathway before proceeding with identifying and cloning the gene.

Screening molecular libraries of chemical compounds for drugs that affect transcriptional regulatory systems has led to important discoveries of great clinical significance. Cyclosporin A (CsA) and FK506, for examples, were selected in standard pharmaceutical screens for inhibition of T-cell activation. It is noteworthy that while these two drugs bind completely different cellular proteins—cyclophilin and FK506 binding protein (FKBP), respectively—the effect of either drug is virtually the same: profound and specific suppression of T-cell activation, phenotypically observable in T cells as inhibition of mRNA production dependent on transcription factors such as NF-AT and NF-KB. Libraries of small peptides have also been successfully screened in vitro in assays for bioactivity. The literature is replete with examples of small peptides capable of modulating a wide variety of signaling pathways. For example, a peptide derived from the HIV-1 envelope protein has been shown to block the action of cellular calmodulin.

A major limitation of conventional in vitro screens is delivery. While only minute amounts of an agent may be necessary to modulate a particular cellular response, delivering such an amount to the requisite subcellular location necessitates exposing the target cell or system to relatively massive concentrations of the agent. The effect of such concentrations may well mask or preclude the targeted response.

Recently, a screening method for identifying regulator molecules was described by Nolan et al. U.S. Pat. No. 6,153,380 (incorporated herein by reference in its entirety). Nolan et al. disclose methods employing, e.g., retroviral vectors to screen random peptides which regulate a desired signaling pathway, thereby resulting in an altered phenotype. The major limitation of using retroviral vectors is that they cannot be employed to study regulatory pathways which result in cessation of cell growth or cell death, either naturally or through genetic engineering.

The present inventors have developed a method that employs a unique poxvirus expression system to efficiently express a library of polynucleotides encoding molecules, e.g., regulator polypeptides or mRNAs, in higher eukaryotic cells such as mammalian cells. The method further provides a means of selecting from this library those molecules that modify a phenotype such as directly or indirectly promoting the transcriptional activation of a target gene, including modified phenotypes resulting in cell death. This method allows efficient selection of regulator molecules, e.g., regulator polypeptides or regulator U1 SnRNAs, that modify a phenotype, for example, through interaction with an unknown or unidentified gene product in a target regulatory pathway. The selected regulator molecule may then serve as a tool to characterize the regulatory pathway which controls the given phenotype.

Thus, it is an object of the present invention to provide improved methods and compositions for the expression of polynucleotide libraries encoding regulator molecules, e.g., regulator polypeptides or regulator U1 SnRNAs, upon introduction into populations of eukaryotic host cells, and improved and expanded methods to select and/or screen for desired regulator molecules in those host cells.

Differentially Expressed Sequences. Cloning, sequencing, and identification of function of mammalian genes is a first priority in a genomic based drug discovery. In particular, it is important to identify and make use of genes which are spatially and/or temporally regulated in an organism, for example, genes involved in differentiation and growth regulation.

Animal model systems such as the fruit fly and the worm are often used in gene identification because of ease of manipulation of the genome and ability to screen for mutants. While these systems have their limitations, large numbers of developmental mutations have been identified in those organisms either by monitoring the phenotypic effects of mutations or by screening for expression of reporter genes incorporated into developmentally regulated genes.

Many features of the mouse make it the best animal model system to study gene function. However, the mouse has not been used for large scale classical genetic mutational analysis because random mutational screening and analysis is very cumbersome and expensive due to long generation times and maintenance costs.

A disadvantage in using animal models for the identification of genes is the need to establish a transgenic animal line for each mutational event. This disadvantage is alleviated in part by using embryonic stem (ES) cell lines because mutational events may be screened in vitro prior to generating an animal. ES cells are totipotent cells isolated from the inner cell mass of the blastocyst. Methods are well known for obtaining ES cells, incorporating genetic material into ES cells, and promotion of differentiation of ES cells. ES cells may be caused to differentiate in vitro or the cells may be incorporated into a developing blastocyst in which the ES cells will contribute to all differentiated tissues of the resulting animal. Vectors for transforming ES cells and suitable genes for use as reporters and selectors are also well known.

Gene entrapment strategies also have been employed to identify developmentally regulated genes. One type of entrapment vector is called a “promoter trap,” which consists of a reporter gene sequence lacking a promoter. Its integration is detected when the reporter is integrated “in-frame” into an exon. In contrast, a “gene trap” vector targets the more prevalent introns of the eucaryotic genome. The latter vector consists of a splice-acceptor site upstream from a reporter gene. Integration of the reporter into an intron results in a fusion transcript containing RNA from the endogenous gene and from the reporter gene sequence.

Gene trap vectors may be made more efficient by incorporation of an internal ribosomal entry site (IRES) such as that derived from the 5′ non-translated region of encephalomyocarditis virus (EMCV). Placement of a IRES site between the splice acceptor and the reporter gene of a gene trap vector means the reporter gene product need not be translated as a fusion product with the endogenous gene product, thereby increasing the likelihood that integration of the vector will result in expression of the reporter gene product.

Gossler, A. et al., Science 244:463-465 (1989) describe the use of enhancer trap gene trap vectors for use in identifying developmentally regulated genes. The gene trap vector consists of the mouse En-2 splice acceptor upstream from lacZ (reporter) and a selector gene (hBa-neo). This and other current methods requires elaborate screening procedures for linking a mutation to a particular spacial/temporal scheme or event whereby the mutation is detected in the relevant tissue.

A more recently developed method is complementation trapping. See WO 99/02719. This method makes use of known genes whose expression is restricted to specific tissue, tissues or specialized cells (“restricted expression”) to facilitate identification and manipulation of new genes and their associated transcription control elements which have similar patterns of expression. The method comprises (i) transforming a eucaryotic cell with a DNA sequence encoding a first indicator component under the control of a promoter having restricted expression; (ii) transforming the cell of (i) or a descendent of the cell of step (i), by operably integrating into the cell's genome DNA lacking a promoter but which comprises a sequence encoding a second indicator component; (iii) producing tissue or specialized cells from the cell of (ii); and (iv) monitoring the tissue or specialized cells of (iii) for a detectable indicator resulting from both the first and second indicator components.

Scott and Craig Current Opinion in Biotechnology 5:40-48 (1994) review random polypeptide libraries. Hupp, T. R. et al., Cell 83:23 7-245 (1995) describe small polypeptides which activate the latent sequence-specific DNA binding function of p53. Palzkill, T. et al., J. Bacteriol. 176:563-568 (1994) report the selection of functional signal cleavage sites from a library of random sequences introduced into TEM-1β-lactamase.

Processing and polyadenylation of precursors to messenger RNA is required for efficient expression of most mammalian genes. Workers at the University of Connecticut have demonstrated that it is possible to interfere with expression of specific sequences at the pre-mRNA level by modifying a 10 bp splice donor site of U1 snRNA to target the terminal exon of a specific gene. Several transgenes including CAT, b-gal and GFP were targeted by stable or transient transfection with specifically modified U1 snRNA. See e.g., Beckley, et al. MoL Cell. Biol 21:2815-2825 (2001). Specific gene expression was reduced by >90% at mRNA and protein level. Importantly, a reduction in the expression of the endogenous bone-specific marker gene osteocalcin by specifically modified U1 snRNA has also been demonstrated. Stable or transient transfection with U1 snRNA modified to target the osteocalcin gene resulted in 80-90% inhibition of osteocaclin mRNA expression in ROS17/2.8 cells.

The inhibitory effect has been shown to be sequence-specific, and the U1 70K protein which binds to an arm of the U1 snRNA was found to play an important role in the inhibition. The mechanism of inhibition is under investigation. Preliminary evidence suggests that there may be an interaction between the 70K protein bound to the modified U1 snRNA and poly(A)polymerase which results in reduced polyadenylation of the targeted gene transcript.

Eukaryotic Expression Libraries. A basic tool in the field of molecular biology is the conversion of poly(A)⁺ mRNA to double-stranded (ds) cDNA, which then can be inserted into a cloning vector and expressed in an appropriate host cell. A method common to many cDNA cloning strategies involves the construction of a “cDNA library” which is a collection of cDNA clones derived from the poly(A)⁺ mRNA derived from a cell of the organism of interest. In a non-limiting example, in order to isolate cDNAs which express immunoglobulin genes, a cDNA library might be prepared from pre B cells, B cells, or plasma cells. Methods of constructing cDNA libraries in different expression vectors, including filamentous bacteriophage, bacteriophage lambda, cosmids, and plasmid vectors, are known. Some commonly used methods are described, for example, in Sambrook et al., Molecular Cloning: A Laboratory Manual, 2d Edition, Cold Spring Harbor Laboratory, publisher, Cold Spring Harbor, N.Y. (1990).

Many different methods of isolating target genes from cDNA libraries have been utilized, with varying success. These include, for example, the use of nucleic acid hybridization probes, which are labeled nucleic acid fragments having sequences complementary to the DNA sequence of the target gene. When this method is applied to cDNA clones in transformed bacterial hosts, colonies or plaques hybridizing strongly to the probe are likely to contain the target DNA sequences. Hybridization methods, however, do not require, and do not measure, whether a particular cDNA clone is expressed. Alternative screening methods rely on expression in the bacterial host, for example, colonies or plaques can be screened by immunoassay for binding to antibodies raised against the protein of interest. Assays for expression in bacterial hosts are often impeded, however, because the protein may not be sufficiently expressed in bacterial hosts, it may be expressed in the wrong conformation, and it may not be processed, and/or transported as it would in a eukaryotic system.

In certain other embodiments, a diverse library may be constructed in vitro using a framework sequence which is common to each member of the library, e.g., a “molecular scaffold,” but in which certain nucleotides and/or amino acids are varied randomly in order to produce the diverse library. When members of the library are expressed in host cells, diverse versions of the molecular scaffold are expressed and can be evaluated for desired properties. Preferably, those nucleotides and/or amino acids which are varied in the molecular scaffold are chosen based on their ability to interact with another molecule of interest, allowing the artisan to select and/or screen for library members which have a desired property. Examples of diverse libraries produced in molecular scaffolds, e.g., polypeptide scaffolds such as an FN3 domain or a nucleotide scaffold such as a U1 SnRNA, are disclosed herein.

Use of mammalian expression libraries to isolate polynucleotides encoding regulator molecules, e.g., regulator polypeptides or regulator U1 SnRNAs, offers several advantages over bacterial libraries. For example, regulator molecules expressed in eukaryotic hosts should be functional and should undergo any normal posttranslational modification. In addition, a protein ordinarily transported through the intracellular membrane system to a desired intracellular compartment or to the cell surface should undergo the complete transport process. Further, use of a eukaryotic system would make it possible to isolate polynucleotides based on functional expression of regulator molecules, e.g., regulator polypeptides or regulator U1 SnRNAs, i.e., their effect on a target cellular process in eukaryotic cells.

With the exception of some recent lymphokine cDNAs isolated by expression in COS cells (Wong, G. G. et al., Science 228:810-815 (1985); Lee, F. et al., Proc. Natl. Acad. Sci. USA 83:2061-2065 (1986); Yokota, T. et al., Proc. Natl. Acad. Sci. USA 83:5894-5898 (1986); Yang, Y. et al., Cell 47:3-10 (1986)), few diverse polynucleotides have been isolated from mammalian expression libraries. There appear to be two principal reasons for this: First, the existing technology (Okayama, H. et al., Mol. Cell. Biol. 2:161-170 (1982)) for construction of large plasmid libraries is difficult to master, and library size rarely approaches that accessible by phage cloning techniques. (Huynh, T. et al., In: DNA Cloning Vol, I, A Practical Approach, Glover, D. M. (ed.), IRL Press, Oxford (1985), pp. 49-78). Second, the existing vectors are, with one exception (Wong, G. G. et al., Science 228:810-815 (1985)), poorly adapted for high level expression. Thus, expression in mammalian hosts previously has been most frequently employed solely as a means of verifying the identity of the protein encoded by a gene isolated by more traditional cloning methods.

Poxvirus Vectors. Poxvirus vectors are used extensively as expression vehicles for protein and antigen expression in eukaryotic cells. The ease of cloning and propagating vaccinia in a variety of host cells has led to the widespread use of poxvirus vectors for expression of foreign protein and as vaccine delivery vehicles (Moss, B., Science 252:1662-7 (1991)).

Large DNA viruses are particularly useful expression vectors for the study of cellular processes as they can express many different proteins in their native form in a variety of cell lines. In addition, gene products expressed in recombinant vaccinia virus have been shown to be efficiently processed and presented in association with MHC class I for stimulation of cytotoxic T cells. The gene of interest is normally cloned in a plasmid under the control of a promoter flanked by sequences homologous to a non-essential region in the virus and the cassette is introduced into the genome via homologous recombination. A panoply of vectors for expression, selection and detection have been devised to accommodate a variety of cloning and expression strategies. However, homologous recombination is an ineffective means of making a recombinant virus in situations requiring the generation of complex libraries or when the insert DNA is large. An alternative strategy for the construction of recombinant genomes relying on direct ligation of viral DNA “arms” to an insert and the subsequent rescue of infectious virus has been explored for the genomes of poxvirus (Merchlinsky et al., Virology 190:522-526 (1992); Pfleiderer et al., J. General Virology 76:2957-2962 (1995); Scheiflinger et al., Proc. Natl. Acad. Sci. USA 89:9977-9981 (1992)), herpesvirus (Rixon et al., J. General Virology 71:2931-2939 (1990)) and baculovirus (Ernst et al., Nucleic Acids Research 22:2855-2856 (1994)).

Poxviruses are ubiquitous vectors for studies in eukaryotic cells as they are easily constructed and engineered to express foreign proteins at high levels. The wide host range of the virus allows one to faithfully express proteins in a variety of cell types. Direct cloning strategies have been devised to extend the scope of applications for poxvirus viral chimeras in which the recombinant genomes are constructed in vitro by direct ligation of DNA fragments to vaccinia “arms” and transfection of the DNA mixture into cells infected with a helper virus (Merchlinsky et al., Virology 190:522-526 (1992); Scheiflinger et al., Proc. Natl. Acad. Sci. USA 89:9977-9981 (1992)). This approach has been used for high level expression of foreign proteins (Pfleiderer et al., J. Gen. Virology 76:2957-2962 (1995)) and to efficiently clone fragments as large as 26 kilobases in length (Merchlinsky et al., Virology 190:522-526 (1992)).

Naked vaccinia virus DNA is not infectious because the virus cannot utilize cellular transcriptional machinery and relies on its own proteins for the synthesis of viral RNA. Previously, temperature sensitive conditional lethal (Merchlinsky et al., Virology 190:522-526 (1992)) or non-homologous poxvirus fowlpox (Scheiflinger et al., Proc. Natl. Acad. Sci. USA 89:9977-9981 (1992)) have been utilized as helper virus for packaging. An ideal helper virus will efficiently generate infectious virus but not replicate in the host cell or recombine with the vaccinia DNA products. Fowlpox virus has the properties of an ideal helper virus as it is used at 37° C., will not revert to a highly replicating strain, and, since it does not recombine with vaccinia DNA or productively infect primate cell lines, can be used at relatively high multiplicity of infection (MOI).

The utility of the vaccinia based direct ligation vector vNotI/tk, has been described by Merchlinsky et al., Virology 190:522-526 (1992). This genome lacks the NotI site normally present in the HindIII F fragment and contains a unique NotI site at the beginning of the thymidine kinase gene in frame with the coding sequence. This allows the insertion of DNA fragments into the NotI site and the identification of recombinant genomes by drug selection. The vNotI/tk vector will only express foreign proteins at the level of the thymidine kinase gene, a weakly expressed gene only made early during viral infection. Thus, the vNotI/tk vector can be used to efficiently clone large DNA fragments but does not fix the orientation of the DNA insert or lead to high expression of the foreign protein.

Customarily, a foreign protein coding sequence is introduced into the poxvirus genome by homologous recombination with infectious virus. In this traditional method, a previously isolated foreign DNA is cloned in a transfer plasmid behind a vaccinia promoter flanked by sequences homologous to a region in the poxvirus which is non-essential for viral replication. The transfer plasmid is introduced into poxvirus-infected cells to allow the transfer plasmid and poxvirus genome to recombine in vivo via homologous recombination. As a result of the homologous recombination, the foreign DNA is transferred to the viral genome.

Although traditional homologous recombination in poxviruses is useful for expression of previously isolated foreign DNA in a poxvirus, the method is not conducive to the construction of libraries, since the overwhelming majority of viruses recovered have not acquired a foreign DNA insert. Using traditional homologous recombination, the recombination efficiency is in the range of approximately 0.1% or less. Thus, the use of poxvirus vectors has been limited to subcloning of previously isolated DNA molecules for the purposes of protein expression and vaccine development.

Alternative methods using direct ligation vectors have been developed to efficiently construct chimeric genomes in situations not readily amenable for homologous recombination (Merchlinsky, M. et al., Virology 190:522-526 (1992); Scheiflinger, F. et al., Proc. Natl. Acad. Sci. USA. 89:9977-9981 (1992). In such protocols, the DNA from the genome is digested, ligated to insert DNA in vitro, and transfected into cells infected with a helper virus (Merchlinsky, M. et al., Virology 190:522-526 (1992), Scheiflinger, F. et al., Proc. Natl. Acad. Sci. USA 89:9977-9981 (1992)). In one protocol, the genome was digested at a unique NotI site and a DNA insert containing elements for selection or detection of the chimeric genome was ligated to the genomic arms (Scheiflinger, F. et al., Proc. Natl. Acad. Sci. USA. 89:9977-9981 (1992)). This direct ligation method was described for the insertion of foreign DNA into the vaccinia virus genome (Pfleiderer et al., J. General Virology 76:2957-2962 (1995)). Alternatively, the vaccinia WR genome was modified by removing the NotI site in the HindIII F fragment and reintroducing a NotI site proximal to the thymidine kinase gene such that insertion of a sequence at this locus disrupts the thymidine kinase gene, allowing isolation of chimeric genomes via use of drug selection (Merchlinsky, M. et al., Virology 190:522-526 (1992)).

The direct ligation vector vNotI/tk allows one to efficiently clone and propagate previously isolated DNA inserts at least 26 kilobase pairs in length (Merchlinsky, M. et al., Virology, 190:522-526 (1992)). Although large DNA fragments are efficiently cloned into the genome, proteins encoded by the DNA insert will only be expressed at the low level corresponding to the thymidine kinase gene, a relatively weakly expressed early class gene in vaccinia. In addition, the DNA will be inserted in both orientations at the NotI site, and therefore might not be expressed at all. Additionally, although the recombination efficiency using direct ligation is higher than that observed with traditional homologous recombination, the resulting titer is relatively low.

Accordingly, poxvirus vectors were previously not used to identify previously unknown genes of interest from a complex population of clones, because a high efficiency, high titer-producing method of cloning did not exist for poxviruses. More recently, however, the present inventor developed a method for generating recombinant poxviruses using tri-molecular recombination. See Zauderer, WO 00/028016, published May 18, 2000, and Zauderer, WO 01/72995, published Oct. 4, 2001, both of which are incorporated herein by reference in their entireties.

Tri-molecular recombination is a novel, high efficiency, high titer-producing method for producing recombinant poxviruses. Using the tri-molecular recombination method in vaccinia virus, the present inventor has achieved recombination efficiencies of at least 90%, and titers at least 2 orders of magnitude higher, than those obtained by direct ligation. According to the tri-molecular recombination method, a poxvirus genome is cleaved to produce two nonhomologous fragments or “arms.” A transfer vector is produced which carries the heterologous insert DNA flanked by regions of homology with the two poxvirus arms. The arms and the transfer vector are delivered into a recipient host cell, allowing the three DNA molecules to recombine in vivo. As a result of the recombination, a single poxvirus genome molecule is produced which comprises each of the two poxvirus arms and the insert DNA.

### SUMMARY OF THE INVENTION

In accordance with one aspect of the present invention, there is provided a method of selecting or identifying polynucleotides which encode regulator molecules, e.g., regulator polypeptides or regulator U1 SnRNAs, which directly or indirectly influence, i.e., induce and/or suppress, the transcriptional activation of a target transcriptional regulatory region, from libraries of polynucleotides expressed in eukaryotic cells.

Also provided is a method of constructing libraries of polynucleotides encoding such regulator molecules in eukaryotic cells using virus vectors, where the libraries are constructed by trimolecular recombination.

Further provided are methods of identifying host cells expressing desired regulator molecules, by selecting and/or screening those host cells for a modified phenotype.

In one aspect of the invention, methods for selecting and/or screening for a regulator molecule capable of modifying a predetermined phenotype of a eukaryotic host cell are provided. The methods comprise the steps of a) providing a population of eukaryotic host cells comprising a target transcriptional regulatory region which is naturally acted upon, i.e., induced and/or suppressed, in a predetermined cellular process, where the target transcriptional regulatory region is operably associated with a gene product, the expression of which produces a detectable modified phenotype; b) introducing a library of polynucleotides encoding a plurality of candidate regulator molecules into the host cells; c) allowing expression of the regulator molecules in the population of host cells; and d) recovering polynucleotides of the library from those host cells which exhibit the modified phenotype, the recovery being either through direct selection or screening. The methods may also include the steps of isolating host cells exhibiting the modified phenotype, or isolating polynucleotides of the library from those host cells which exhibit the modified phenotype.

The invention further provides methods for identifying and/or isolating previously unknown target molecules or regulatory regions associated with the regulatory pathway of interest, using either an isolated polynucleotide which encodes a desired regulator molecule identified as described herein, or the expression product of that polynucleotide.

Regulator molecules encoded by polynucleotide libraries of the present invention typically comprise a “candidate molecule,” either a candidate peptide or a candidate polynucleotide, e.g., RNA, which is diverse among members of the library, and a “molecular scaffold” fused to the candidate molecule such that said candidate molecule is displayed in a manner sufficient to exert a regulator function. For example, candidate peptides are displayed on surface areas of a scaffold polypeptide, and a candidate RNA might be displayed as the initial 10-bp segment of a U1 SnRNA scaffold.

Additionally, polynucleotides contained in the library may be associated with heterologous polynucleotides which may encode heterologous polypeptides encoding, for example, fusion partners of regulatory polypeptides.

Furthermore, the present invention provides molecular libraries of virus vectors, preferably poxvirus vectors, more preferably vaccinia virus vectors, comprising, in operable configuration, polynucleotides encoding regulator molecules as described herein, and cellular libraries containing the viral libraries. In further aspects, the present invention provides kits to screen for regulator molecules, using the methods and compositions described herein, desired regulator molecules isolated by the methods described herein, and compositions comprising those regulator molecules.

### BRIEF DESCRIPTION OF THE FIGURES

FIG. 1. Nucleotide Sequence of p7.5/tk and pEL/tk. The nucleotide sequence of the promoter and beginning of the thymidine kinase gene for v7.5/tk and vEL/tk.

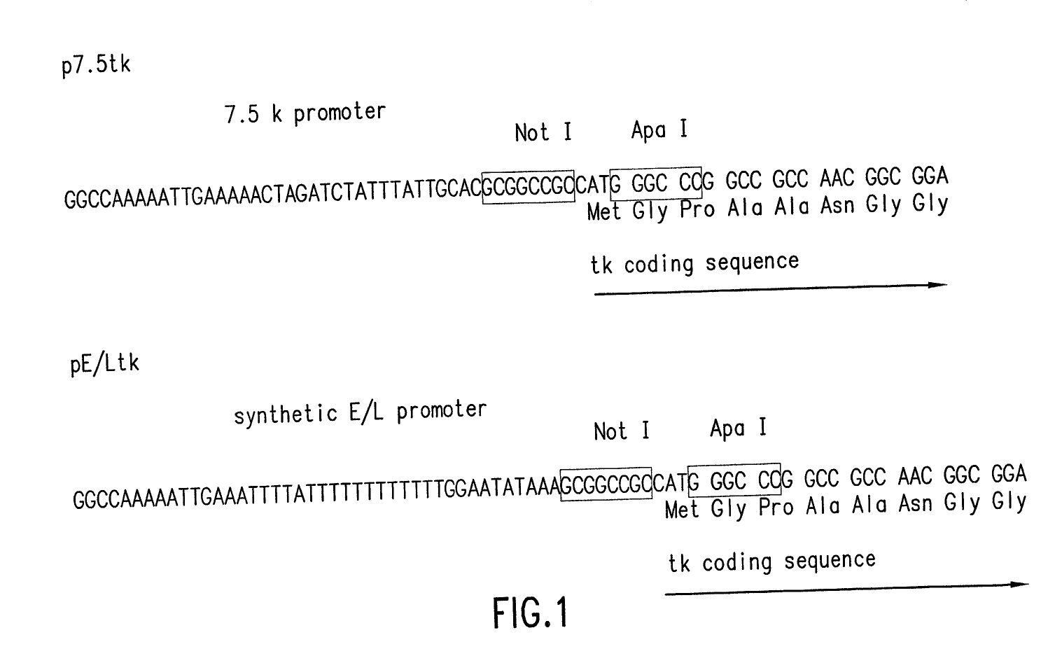

FIG. 2. Schematic of the Tri-Molecular Recombination Method.

FIG. 3. Construction of p7.5/FN3-BC Random/tk Library.

FIG. 4. Construction of p7.5/FN3-BC/FG Random/tk.

FIG. 5. Attenuation of poxvirus-mediated cytopathic effects.

### DETAILED DESCRIPTION OF THE INVENTION

The present invention is broadly directed to methods of identifying and/or producing regulator molecules, e.g., regulator polypeptides or regulator U1 SnRNAs, which directly or indirectly influence, i.e., induce and/or suppress, the transcriptional activation of a target transcriptional regulatory region in a eukaryotic cell. In addition, the invention is directed to methods of identifying polynucleotides which encode regulator molecules from complex expression libraries of polynucleotides encoding such regulator molecules, where the libraries are constructed and screened in eukaryotic host cells. Further embodiments include an isolated regulator molecule produced by any of the above methods, compositions comprising isolated regulator molecules, a library of polynucleotides constructed to express candidate regulator molecules, and a kit allowing production of such regulator molecules.

A particularly preferred aspect of the present invention is the construction of complex polynucleotide libraries from which to isolate polynucleotides encoding desired regulator molecules in eukaryotic host cells using poxvirus vectors constructed by trimolecular recombination. The ability to construct complex libraries in a pox virus based vector and to select and/or screen for specific recombinants on the basis of a modified phenotype, in particular a modified phenotype resulting in cell death, can be the basis for identification of desired regulator molecules in eukaryotic cells, in particular, human cells. It would overcome the limitations of synthesis and assembly of polynucleotide libraries in bacteria or yeast, and limitations of screening or selection schemes afforded by retrovirus vectors.

It is to be noted that the term “phenotype” refers to the total physical and biochemical characteristics displayed by host cells under a particular set of environmental factors, regardless of the actual genotype of the organism. The term “modified phenotype” refers to a change in the form, character, or intensity of a physical or biochemical characteristic displayed by host cells under a particular set of environmental factors. A phenotype might be displayed by a given host cell in response to any number of environmental factors including, but not limited to temperature, exposure to certain molecules, or signalling by and extracellular molecule (e.g., a hormone), or another cell. In certain embodiments a given predetermined phenotype, and any modifications of that phenotype may be those which occur naturally in a given host cell. In alternative embodiments, a host cell is engineered such that a more easily detectable phenotype is substituted into a transcriptional pathway of interest. For example, a reporter gene may be inserted in operable association with a promoter in a cellular regulatory pathway of interest. In either case, it is preferred that the phenotype of interest, and any modifications of that phenotype that are contemplated, are “predetermined,” i.e., they are known and well characterized, and are readily detectable in the host cell used to screen and/or select for regulator molecules thereof of the present invention.

Furthermore, a regulator molecule of the present invention is selected and/or screened for by its ability to “induce” and/or “suppress” transcriptional activation of a target transcriptional regulatory region in a eukaryotic host cell. In this context, the term “induce” is used herein to describe the ability of the desired regulator molecule to effect, either directly or indirectly, transcriptional activation, e.g., initiation of transcription in a given regulatory pathway which results, at its end point, in the display of a modified phenotype. The term “suppress” is used herein to describe the ability of the desired regulator molecule to block or reduce, either directly or indirectly, transcriptional activation, e.g., to stop transcription in a given regulatory pathway which is normally activated, where the absence of that activation results in the display of a modified phenotype. Thus, the action of the desired regulator molecule, on the given phenotype may be direct, for example, activating or suppressing transcription of the gene product which determines the modified phenotype, or indirect, for example, activating or suppressing expression of a gene in a signal transduction pathway which is far removed from the actual gene product responsible for the modified phenotype.

It is to be noted that the term “a” or “an” entity, refers to one or more of that entity; for example, “a regulator molecule,” is understood to represent one or more regulator molecules. As such, the terms “a” (or “an”), “one or more,” and “at least one” can be used interchangeably herein.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject.

The terms “tissue culture” or “cell culture” or “culture” or “culturing” refer to the maintenance or growth of cells, e.g., plant or animal tissue or cells in vitro under conditions that allow preservation of cell architecture, preservation of cell function, further differentiation, or all three. “Primary tissue cells” are those taken directly from tissue, i.e., a population of cells of the same kind performing the same function in an organism. Treating such tissue cells with the proteolytic enzyme trypsin, for example, dissociates them into individual primary tissue cells that grow or maintain cell architecture when seeded onto culture plates. Cell cultures arising from multiplication of primary cells in tissue culture are called “secondary cell cultures.” Most secondary cells divide a finite number of times and then die. A few secondary cells, however, may pass through this “crisis period,” after which they are able to multiply indefinitely to form a continuous “cell line.” The liquid medium in which cells are cultured is referred to herein as “culture medium” or “culture media.”

The term “polynucleotide” refers to any one or more nucleic acid segments, or nucleic acid molecules, e.g., DNA or RNA fragments, present in a nucleic acid or construct. A “polynucleotide encoding a regulator molecule” refers to a polynucleotide which comprises the coding region for such a molecule, e.g., comprises the coding region for a regulator polypeptide or a regulator U1 SnRNA. In addition, a polynucleotide may comprise additional nucleic acid segments which encode regulatory elements such as a promoter or a transcription terminator, or may encode a specific element of a polypeptide or protein, such as a secretory signal peptide or a functional domain. As used herein, the term “identify” and grammatical equivalents refers to methods in which desired molecules, e.g., polynucleotides encoding regulator molecules, are distinguished from a plurality or library of such molecules. Identification methods include “selection” and “screening.” As used herein, “selection” methods are those in which the desired molecules may be directly separated from other candidate molecules in the library. For example, in one selection method described herein, host cells comprising the desired polynucleotides are directly separated from the host cells comprising the remainder of the library by becoming nonadherent, e.g., undergoing a lytic event, and thereby being released from the substrate to which the remainder of the host cells are attached. For another example, FACS (fluorescence-activated cells sorting) is used to separate cells exhibiting the modified phenotype from the remainder of the host cells which do not exhibit the modified phenotype. As used herein, “screening” methods are those in which pools comprising the host cells are subjected to an assay in which the modified phenotype can be detected. For example, aliquots of the pools containing host cells which exhibit the modified phenotype may then divided into successively smaller pools which are likewise assayed, until a pool which is highly enriched for those host cells is achieved.

The present invention provides methods and compositions to create, effectively introduce into cells and select and/or screen for compounds that influence a target transcriptional regulatory region which is naturally induced in a target cellular process. Little or no knowledge of the target regulatory region is required, other than a presumed signaling event and a detectable modified phenotype in the host cell. The disclosed methods are an in vivo strategy for accessing intracellular regulatory mechanisms. The invention also provides for the isolation of the constituents of the pathway, the tools to characterize the pathway, and lead compounds for pharmaceutical development.

By “target transcriptional regulatory region” is meant a component of the transcriptional network of a cell which functions in producing a “target cellular process.” A target transcriptional regulatory region of interest may include, for instance, a promoter to direct mRNA transcription, and/or a transcriptional terminator to direct where transcription should end. In general, target transcriptional regulatory regions may include sites for transcription initiation and termination, and, in the transcribed region, a ribosome binding site for translation.

In addition, target transcriptional regulatory regions of interest may include control regions that regulate as well as engender expression. Generally, such regions will operate by controlling transcription, such as repressor binding sites, activator binding sites, enhancers, silencers, or insulators, among others. Enhancers are regions of DNA which bind certain transcription factors (“Enhancer-binding protein”) to enhance transcription, and may be thousands of base pairs away from the gene they control. Binding increases the rate of transcription of the gene. Enhancers can be located upstream, downstream, or even within the gene they control. Silencers are control regions of DNA that, like enhancers, may be located thousands of base pairs away from the gene they control. However, when transcription factors bind to them, expression of the gene they control is repressed. Insulators are stretches of DNA ,as few as 42 base pairs, located between enhancer(s) and promoter or silencer(s) and promoter of adjacent genes. Their function is to prevent a gene from being influenced by the activation (or repression) of its neighbors.

“Target transcriptional regulatory regions” of the invention should be distinguished from “vector transcriptional regulatory regions,” which are those regulatory regions engineered in a vector to deliver and express candidate regulator molecules to suitable host cells. Vector transcriptional regulatory regions include, e.g., promoters, enhancers, silencers, and transcriptional terminators which are part of a eukaryotic virus vector of the invention. Vector transcriptional regulatory regions may be heterologous to the virus vector being used, or may be native and specific to the virus vector being used. Of particular interest in the present invention are “poxvirus transcriptional regulatory regions.” Poxvirus gene expression and replication takes place entirely in the cytoplasm of infected cells. Accordingly, poxviruses utilize promoters, RNA polymerases, DNA polymerases, and transcriptional termination regions which are different from usual eukaryotic genes which are transcribed in the nucleus.

As used herein, a “target cellular process” is a series of events in a cell leading up to known phenotype. A target cellular process can be relatively simple, for example, it may involve expression of a single gene, the product of which allows display of a phenotype of interest in a cell. Often, a target cellular process is more complex, e.g., involving expression of transcriptional regulatory factors, e.g., inducers or inhibitors, which in turn activate or suppress transcription of another gene which ultimately results in display of the phenotype of interest. The expression of regulatory factors is often caused by external, environmental stimuli acting on the cell.

A example of such a target cellular process is a “signal transduction pathway.” Signal transduction at the cellular level refers to the movement of signals from outside the cell to inside. The movement of signals can be simple, like that associated with receptor molecules of the acetylcholine class: receptors that constitute channels which, upon ligand interaction, allow signals to be passed in the form of small ion movement, either into or out of the cell. These ion movements result in changes in the electrical potential of the cells that, in turn, propagates the signal along the cell. More complex signal transduction involves the coupling of ligand-receptor interactions to many intracellular events. These events include phosphorylations by tyrosine kinases and/or serine/threonine kinases. Protein phosphorylations change enzyme activities and protein conformations. The eventual outcome is an alteration in cellular activity and changes in the program of genes expressed within the responding cells, ultimately resulting in display of the phenotype of interest. In relation to the present invention, while the “endpoint” gene product is often known, it is often difficult for those of ordinary skill in the art to identify and characterize the initial or intermediate steps in signal transduction pathways, or other target cellular processes. Regulator molecules of the present invention are identified based on a phenotype displayed as the end product, but in many cases the regulator molecules identified will actually interact at an unknown target transcriptional regulatory region that is further up in the pathway. Isolated regulator molecules so identified can then be used to further characterize individual steps in the target cellular process.

The present invention provides methods for the selection and/or screening of candidate regulator molecules, e.g., regulator polypeptides or regulator U1 SnRNAs, which are capable of altering the phenotype of cells containing the molecules. The methods of the present invention provide a significant improvement over conventional screening techniques, as they allow the rapid selection and/or screening of desired regulator molecules from large numbers of candidate regulator molecules and their corresponding expression products in a single, in vivo step. Thus, by delivering a library of candidate regulator molecules to a population of host cells and selecting and/or screening for the desired regulator molecules in the same cells, without the need to collect or synthesize in vitro the candidate regulator molecules, highly efficient recovery of polynucleotides encoding desired regulator molecules is accomplished. In addition, the present methods allow selection and/or screening for molecules which can regulate a desired cellular pathway in the absence of significant prior characterization of the cellular pathway per se.

Thus, the present invention provides methods for screening candidate regulator molecules, e.g., regulator polypeptides or regulator U1 SnRNAs, for those capable inducing or suppressing activation of transcription, thereby altering the phenotype of a cell.

By “candidate regulator molecules,” “candidate regulator polypeptides,” “candidate regulator RNAs,” “candidate molecules,” “candidate drugs” or “candidate expression products” or grammatical equivalents herein is meant the expression product of a candidate polynucleotide or candidate nucleic acid, which may be tested for the ability to alter the phenotype of a cell. As is described below, candidate regulator molecules are the expression products of candidate polynucleotides, and encompass several chemical classes, including polypeptides and nucleic acids such as DNA, messenger RNA (mRNA), antisense RNA, nuclear RNA, e.g., U1 SnRNA, transfer RNA, ribosomal RNA, ribozyme components, etc. Thus, the candidate regulator molecules (expression products) may be either translation products of the candidate nucleic acids, i.e., polypeptides, or transcription products of the candidate nucleic acids, i.e., either DNA or RNA.

In one embodiment, the candidate regulator molecules are candidate regulator polypeptides, which are translation products of the candidate polynucleotides. In this embodiment, candidate polynucleotides are introduced into the cells, and the cells express the nucleic acids to form polypeptides. Generally, candidate regulator polypeptides of the present invention comprise at least two components, a “candidate peptide” and a “molecular scaffold.” Molecular scaffolds, as defined below, maintain the candidate peptides in a conformationally restricted or stable form. Candidate peptides are preferably situated on an exposed surface of a molecular scaffold protein, such that the candidate peptides can interact with cellular elements. A regulator polypeptide may comprise one or more candidate peptides, each ranging from about 2 amino acids in length to about 100 amino acids may be used, with polypeptides ranging from about 2 to about 50 being preferred, with from about 2 to about 30 being particularly preferred and from about 2 to about 10 being especially preferred. Generally, polypeptides may be about 2,3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the candidate regulator polypeptides are transcription products of the candidate nucleic acids, and are thus also nucleic acids including mRNA, antisense RNA, nuclear RNAs, e.g., U1 SnRNA, and ribozymes or portions thereof. Generally, regulator RNAs, similar to regulator polypeptides, will comprises at least two components, a “candidate RNA fragment” and a “molecular scaffold.” Candidate RNA fragments are preferably situated on an exposed portion of a molecular scaffold protein, such that the candidate RNA fragments can interact with cellular elements.

By “molecular scaffold” or grammatical equivalents herein is meant a sequence, which, as part of a candidate regulator molecule, causes a candidate peptide or RNA fragment to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the polypeptide with the target protein. This fact has been recognized in the combinatorial library generation systems using biologically generated short peptides in bacterial phage systems. A number of workers have constructed small domain molecules in which one might present randomized peptide structures.

While the candidate regulator molecules may be either nucleic acid or peptides, in certain embodiments the molecular scaffolds are preferably used with polypeptide candidate molecules. Thus, synthetic molecular scaffolds, i.e. artificial polypeptides, are capable of presenting a randomized candidate peptide as a conformationally-restricted domain. Generally such molecular scaffolds comprise a first portion joined to the N-terminal end of the randomized candidate peptide, and a second portion joined to the C-terminal end of the candidate peptide; that is, the peptide is inserted into the molecular scaffold, although variations may be made, as outlined below. To increase the functional isolation of the randomized expression product, the molecular scaffolds are selected or designed to have minimal biological activity when expressed in the target cell.

Preferred molecular scaffolds maximize accessibility to the peptide by presenting it on an exterior loop. Accordingly, suitable molecular scaffolds include, but are not limited to, immunoglobulin-based structures such as minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

In one embodiment, the molecular scaffold is a coiled-coil structure, allowing the presentation of the randomized peptide on an exterior loop. See, for example, Myszka et al., Biochem. 33:2362-2373 (1994), hereby incorporated by reference. Using this system investigators have isolated peptides capable of high affinity interaction with the appropriate target. In general, coiled-coil structures allow for between 6 to 20 randomized positions.

An example of a coiled-coil molecular scaffold is as follows: MGCAALESEVSALESEVASLESEVAALGRGDMPLAAVKSKLSAVKSK LASVKSKLAA CGPP (SEQ ID NO:1). The underlined regions represent a coiled-coil leucine zipper region defined previously (see Martin et al., EMBO J. 13(22):5303-5309 (1994), incorporated by reference). The bolded GRGDMP (SEQ ID NO:2) region represents the loop structure and when appropriately replaced with randomized peptides (i.e. candidate regulator peptides) can be of variable length.

In another embodiment, the molecular scaffold is a minibody structure. A “minibody” is essentially composed of a minimal antibody complementarity region. The minibody molecular scaffold generally provides two randomizing regions that in the folded protein are presented along a single face of the tertiary structure. See for example Bianchi et al.,J. Mol. Biol. 236(2):649-59 (1994), and references cited therein, all of which are incorporated by reference). Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

An example of a minibody molecular scaffold is as follows: MGRNSQATSGFTFSHFYMEWVRGGEYIAASRHKHNKYTTEYSASVK GRYIVSRDTS QSILYLQKKKGPP (SEQ ID NO:3). The bold, underlined regions are the regions which may be randomized. The italized phenylalanine must be invariant in the first randomizing region. The entire polypeptide is cloned in a three-oligonucleotide variation of the coiled-coil embodiment, thus allowing two different randomizing regions to be incorporated simultaneously. This embodiment utilizes non-palindromic BstXI sites on the termini.

In yet another embodiment, the molecular scaffold is a a fibronectin type III domain (FN3) as described in Koide, A. et al., J. Mol. Biol. 284:1141-1151 (1998), which is incorporated herein by reference in its entirety. Several regions of this molecular scaffold may be randomized, in particular the BC loop the FG loop and the terminal tail. Preferred regions to randomize include the BC loop and the FG loop, as described in the examples, infra.

In yet another embodiment, the molecular scaffold is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective molecular scaffolds may be generated by the random regions themselves. For example, the random regions may be “doped” with cysteine residues which, under the appropriate redox conditions, may result in highly crosslinked structured conformations, similar to a molecular scaffold. Similarly, the randomization regions may be controlled to contain a certain number of residues to confer β-sheet or α-helical structures.

At a minimum, candidate regulator molecules comprise a molecular scaffold and randomized expression products of the candidate polynucleotides. That is, every candidate regulator molecule has a randomized portion, as defined below, that is the basis of the selection and/or screening methods outlined herein. In addition to the randomized portion, the candidate regulator molecules may also include additional elements encoded by heterologous polynucleotides, for example, fusion partners.

Heterologous sequences. Each polynucleotides encoding a candidate regulator molecule, may further comprise a heterologous sequence upstream of or downstream from the sequence encoding the regulator molecule. As such, it is noted that the term “heterologous sequence” may include a heterologous polynucleotide sequence, may be located upstream or downstream of the polynucleotide sequence encoding the regulator molecule, and the heterologous sequence may be in operable association with the polynucleotide sequence encoding the regulator molecule. Furthermore, where the regulator molecule is a regulator polypeptide as described herein, the heterologous polynucleotide sequence may encode a heterologous polypeptide, which may be fused, either upstream or downstream or at both ends, to the regulator polypeptide. Generally, if a “heterologous polynucleotide” is associated with a library of polynucleotides encoding candidate regulator molecules, each individual member of the library will comprise the same heterologous polynucleotide.

Some preferred heterologous polypeptides are disclosed in U.S. Pat. No. 6,153,380, which is incorporated herein by reference in its entirety. For example, in a preferred embodiment, candidate regulator polypeptides comprise a targeting sequence capable of constitutively localizing the regulator molecule, to a predetermined cellular locale, including subcellular locations such as the golgi, endoplasmic reticulum, nucleus, nucleoli, nuclear membrane, mitochondria, chloroplast, secretory vesicles, lysosome, and cellular membrane.

In certain embodiments, candidate regulator polypeptides comprise a heterologous polynucleotide encoding a fusion partner. By “fusion partner” or “functional group” herein is meant a sequence that is associated with the candidate peptide, that confers upon all members of the library in that class a common function or ability. Fusion partners can be heterologous (i.e. not native to the host cell), or synthetic (not native to any cell). Suitable fusion partners include, but are not limited to: a) targeting sequences, defined below, which allow the localization of the candidate regulator polypeptide into a subcellular or extracellular compartment; b) rescue sequences as defined below, which allow the purification or isolation of either the candidate regulator polypeptides or the nucleic acids encoding them; c) stability sequences, which confer stability or protection from degradation to the candidate regulator polypeptide or the nucleic acid encoding it, for example resistance to proteolytic degradation; d) dimerization sequences, to allow for polypeptide dimerization; or e) any combination of a), b), c), and d), as well as linker sequences as needed.

In a preferred embodiment, the fusion partner is a targeting sequence. As will be appreciated by those in the art, the localization of proteins within a cell is a simple method for increasing effective concentration and determining function. For example, RAF1 when localized to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signalling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val) (SEQ ID NO:4), Kalderon et al., Cell 39:499-509 (1984); the human retionic acid receptor-β nuclear localization signal (ARRRRP) (SEQ ID NO:5); NFKB p50 (EEVQRKRQKL (SEQ ID NO:6); Ghosh et al., Cell 62:1019 (1990); NFKB p65 (EEKRKRTYE (SEQ ID NO:7); Nolan et al., Cell 64:961 (1991); and others (see for example Boulikas T. J. Cell. Biochem. 55(4):32-58 (1994)) and double basic NLS's exemplified by that of the Xenopus protein, nucleoplasmin (Ala Val LysArg ProAla AlaThr Lys Lys Ala Gly Gln Ala Lys Lys Lys Lys LeuAsp) (SEQ ID NO:8), Dingwall et al., Cell30:449-458, (1982) and Dingwall et al., J. Cell Biol. 107:641-849 (1988)). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall and Laskey Ann. Rev. Cell Biol. 2:367-390 (1986); Bonnerof et al., Proc. Natl. Acad. Sci. USA 84:6795-6799 (1987); Galileo et al., Proc. Natl. Acad. Sci. USA 87:458-462 (1990)).

In a preferred embodiment, the targeting sequence is a membrane anchoring signal sequence. This is useful since many parasites and pathogens bind to the membrane, in addition to the fact that many intracellular events originate at the plasma membrane. Thus, membrane bound libraries are useful for both the identification of important elements in these processes as well as for the discovery of effective inhibitors.

Membrane-anchoring sequences are well known in the art and are based on the genetic geometry of mammalian transmembrane molecules. Peptides are introduced into the membrane based on a signal sequence (designated herein as ssTM) and require a hydrophobic transmembrane domain (herein TM). The transmembrane proteins are introduced into the membrane such that the regions encoded 3′ of the transmembrane domain are intracellular and the sequences 5′ become extracellular. In preferred embodiment, the transmembrane domains are placed 5′ of the Ig or Ig fragment they will serve to anchor it as an intracellular domain. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al., Eur. J. Biochem. 174:671 (1988)) and insulin receptor beta-chain (residues 1-27 are the signal,957-959, are the transmembrane domain and 960-1382 are the cytoplasmic domain; see Hatakeyama supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP (SEQ ID NO:9); Nakauchi et al., Proc. Natl. Acad. Sci. USA 82:5126 (1985) and 1-21 in the case of ICAM-2 (MSSFGYRTLTVALFTLICCPG (SEQ ID NO:10); Staunton et al., Nature (London) 339:61 (1989)). These leader sequences deliver the construct to the membrane while the hydrophobic transmembrane domains, placed 5′ or 3′ of the Ig or Ig fragment, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR (SEQ ID NO:11); Nakauchi supra) and 224-256 from ICAM-2 (MVIIVTVVSVLLSLFVTSVLLCFIFGQHLRQQR (SEQ ID NO:12); Staunton, supra).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT (SEQ ID NO:13); see Homans et al., Nature 333(6170):269-72 (1988), and Moran et al., J. Biol. Chem. 266:1250 (1991)). In order to do this, the GPI sequence from Thy-1 can be cassetted 3′ of the Ig or Ig fragment in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (SEQ ID NO:14) (see Cross et al., Mol. Cell. Biol. 4(9):1834 (1984); Spencer et al., Science 262:1019-1024 (1993)). This motif has already been shown to be effective in the localization of reporter genes and can be used to anchor the zeta chain of the TCR. This motif is placed 5′ of the Ig or Ig fragment in order to localize the construct to the plasma membrane. Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL (SEQ ID NO:15); Stoffel et al, J. Biol. Chem 269:27791 (1994)); from rhodopsin (KQFRNCMLTSLCCGKNPLGD (SEQ ID NO:16); Barnstable et al., J. Mol. Neurosci. 5(3):207 (1994)); and the p21 II-ras 1 protein (LNPPDESGPGCMSCKCVLS (SEQ ID NO:17); Capon et al., Nature 302:33 (1983)).

In a preferred embodiment, the targeting sequence is a lysozomal targeting sequence, including, for example, a lysosomal degradation sequence such as Lamp-2 (KFERQ (SEQ ID NO:18); Dice Ann. N.Y. Acad. Sci. 674:58 (1992); or lysosomal membrane sequences from Lamp-I (MLIPIAGFFALAGLVLIVLIAYLIGRKRSHAGYQTI (SEQ ID NO:19), Uthayakumar et al., Cell. Mol. Biol. Res. 41:405 (1995)) or Lamp-2 (LVPIAVGAALAGVLILVLLAYFIGLKHHHAGYEOF (SEQ ID NO:20), Konecki et al., Biochem. Biophys. Res. Comm. 205:1-5 (1994), both of which show the transmembrane domains in italics and the cytoplasmic targeting signal underlined.

Alternatively, the targeting sequence may be a mitochondrial localization sequence, including mitochondrial matrix sequences (e.g. yeast alcohol dehydrogenase III; MLRTSSLFTRRVQPSLFSRNILRLQST (SEQ ID NO:21); Schatz Eur. J. Biochem. 165:1-6 (1987)); mitochondrial inner membrane sequences (yeast cytochrome c oxidase subunit IV, MLSLRQSIRFFKPATRTLCSSRYLL (SEQ ID NO:22); Schatz, supra); mitochondrial intermembrame space sequences (yeast cytochrome c1; M F S M L S K R W A Q R-TLSKSFYSTATGAASKSGKLTQKLVTAGVMAGITASTLLYADSLTAEA MTA (SEQ ID NO:23); Schatz, supra) or mitochondrial outer membrane sequences (yeast 70 kD outer membrane protein; MKSFITRNKTAILATVAATGTAIGAYYYYNQLQQQQQRGKK (SEQ ID

NO:24); Schatz, supra).

The target sequence may also be an endoplasmic reticulum sequence, including the sequences from calreticulin (KDEL (SEQ ID NO:25); Pelham, Royal Society London Transactions B; 1-10 (1992)) or adenovirus E3/19K protein (LYLSRRSFIDEKKMP (SEQ ID NO:26); Jackson et al., EMBO J. 9:3153 (1990).

Furthermore, targeting sequences also include peroxisome sequences (for example, the peroxisome matrix sequence from Luciferase; SYL; Keller et al., Proc. Natl. Acad. Sci. USA 4:3264 (1987)); famesylation sequences (for example, P21 H-ras 1; LNPPDESGPGCMSCKCVLS (SEQ ID NO:27), Capon, supra); geranylgeranylation sequences (for example, protein rab-5A; LTEPTQPTRNQCCSN (SEQ ID NO:28); Farnsworth Proc. Natl. Acad. Sci. USA 91:11963 (1994)); or destruction sequences (cyclin B1; RTALGDIGN (SEQ ID NO:29); Klotzbucher et al., EMBO J. 1:3053 (1996)).

In one embodiment, the targeting sequence is a secretory signal sequence capable of effecting the secretion of the regulator polypeptide. This approach is particularly suitable for synthesizing regulator polypeptides, from polynucleotides isolated by the methods of the invention, when the isolated regulator polypeptide is to be used in further experiments, for example, to further characterize a target transcriptional regulatory region, or for therapeutic purposes. There are a large number of known secretory signal sequences which are placed 5′ to the regulator polypeptide, and are cleaved to effect secretion through a cellular membrane. Secretory signal sequences and their transferability to unrelated proteins are well known, e.g., Silhavy et al., Microbiol. Rev. 49:398-418 (1985).

Suitable secretory sequences are known, including signals from IL-2 (MYRMQLLSCIALSLALVTNS (SEQ ID NO:30); Villinger et al., J. Immunol. 155:3946 (1995)), growth hormone (MATGSRTSLLLAFGLLCLPWLQEGSAFPT (SEQ ID NO:31); Roskam et al., Nucleic Acids Res. 7:30 (1979)); preproinsulin (MALWMRLLPLLALLALWGPDPAAA FVN (SEQ ID NO:32); Bell et al., Nature 284:26 (1980)); and influenza HA protein (MKAKLLVLLYAFVAGDQI (SEQ ID NO:33); Sekiwawa et al., Proc. Natl. Acad. Sci. 80:3563)), with cleavage between the non-underlined-underlined junction. A particularly preferred secretory signal sequence is the signal leader sequence from the secreted cytokine IL4, which comprises the first 24 amino acids of IL-4 as follows: MGLTSQLLPPLFFLLACAGNFVHG (SEQ ID NO:34).

In certain embodiments, the fusion partner of a candidate regulator polypeptide is a rescue sequence. A rescue sequence is a sequence which may be used to purify or isolate either the regulator polypeptide, or the polynucleotide encoding it. Thus, for example, peptide rescue sequences include purification sequences such as the 6-His (SEQ ID NO:26) tag for use with Ni affinity columns and epitope tags for detection, immunoprecipitation, or FACS (fluorescence-activated cell sorting). Suitable epitope tags include myc (for use with commercially available 9E10 antibody), the BSP biotinylation target sequence (a short peptide sequence that binds to bacterial enzyme BirA), influenza tags (for example, those that are derived from nucleoprotein or hemagglutinin proteins of influenza virus), LacZ (β-galactosidase) or active fragments thereof, and GST (glutathione S-transferase) or active fragment thereof. Suitable epitope tags also include any detectable fragments of any known epitope tags.

In certain embodiments, combinations of heterologous polypeptide fusion partners are used. Thus, for example, any number of combinations of targeting sequences, secretory sequences, rescue sequences, and stability sequences may be used, with or without linker sequences. One can cassette in various fusion polynucleotides encoding heterologous polypeptides 5′ and 3 of the regulator polypeptide-encoding polynucleotide. Table 1 outlines some of the possible combinations as follows. Using Rp as the candidate regulator polypeptide, and representing each targeting sequence by another letter, (e.g. N for nuclear localization sequence) each construct can be named as a string of representative letters reading N-terminal to C-terminal as protein, such as N Rp or if cloned downstream of the regulator polypeptide-encoding polynucleotide, Rp N. As implied here, the heterologous sequences are cloned as cassettes into sites on either side of the polynucleotide encoding the regulator polypeptide. C is for cytoplasmic (e.g. no localization sequence), E is a rescue sequence such as the myc epitope, G is a linker sequence (G10 is a glycine-serine chain of 10 amino acids, and G20 is a glycine-serine chain of 20 amino acids), M is a myristylation sequence, N is a nuclear localization sequence, ssTM is the signal sequence for a transmembrane anchoring sequence, TM is the transmembrane anchoring sequence, GPI is a GPI membrane anchor sequence; S is a secretory signal sequence, etc. As will be appreciated by those in the art, any number of combinations can be made, in addition to those listed below.

TABLE 1

| | |
|---|---|
| cytoplasmic | C Rp |
| | C E Rp |
| | C Rp E |
| secreted | S Rp |
| | S E Rp |
| | S Rp E |
| myristylated | M Rp |
| | M E Rp |
| | M GE20 Rp |
| transmembrane | ssTM Rp |
| (intracellular) | ssTM Rp TM |
| | ssTM Rp E TM |
| | ssTM Rp G20 E TM |
| | ssTM Rp E |
| transmembrane (GPI linked) | ssTM Rp G E TM |
| nuclear localization | N E Rp |
| | N Rp E |

As will be appreciated by those in the art, these modules of sequences can be used in a large number of combinations and variations.

Localization signals as described herein can be located anywhere on the regulator polypeptide so long as the signal is exposed in the regulator polypeptide and its placement does not disrupt the ability of the candidate peptide to bind to cellular elements or the ability of the ability of the molecular scaffold to assume a constrained conformation. For example, it can be placed at the carboxy or amino terminus or anywhere within the regulator polypeptide, providing it satisfies the above conditions.

Additional heterologous polypeptide fusion partners include the following from WO 94/02610 and WO 99/14353, the disclosures of which are incorporated herein by reference in their entireties: For example, signals such as Lys Asp Glu Leu (SEQ ID NO:35) [Munro et al., Cell 48:899-907 (1987)] Asp Asp Glu Leu (SEQ ID NO:36), Asp Glu Glu Leu (SEQ ID NO:37), Gln Glu Asp Leu (SEQ ID NO:38) and Arg Asp Glu Leu (SEQ ID NO:39) [Hangejorden et al., J. Biol. Chem. 266:6015 (1991), for the endoplasmic reticulum; Pro Lys Lys Lys Arg Lys Val (SEQ ID NO:40) [Lanford et al., Cell 46:575 (1986)] Pro Gln Lys Lys Ile Lys Ser (SEQ ID NO:41) [Stanton, L. W. et al., Proc. Natl. Acad. Sci USA 83:1772 (1986); Gln Pro Lys Lys Pro (SEQ ID NO:42) [Harlow et al., Mol. Cell Biol. 5:1605 (1985)], Arg Lys Lys Arg (SEQ ID NO:43), for the nucleus; and Arg Lys Lys Arg Arg Gln Arg Arg Arg Ala His Gln (SEQ ID NO:44), [Seomi et al., J. Virology 64:1803 (1990)], Arg Gln Ala Arg Arg Asn Arg Arg Arg Arg Trp Arg Glu Arg Gln Arg (SEQ ID NO:45) [Kubota et al., Biochem. and Biophys, Res. Comm. 162:963 (1989)], Met Pro Leu Thr Arg Arg Arg Pro Ala Ala Ser Gln Ala Leu Ala Pro Pro Thr Pro (SEQ ID NO:46) [Siomi et al., Cell 55:197 (1988)] for the nucleolar region; Met Asp Asp Gln Arg Asp Leu Ile Ser Asn Asn Glu Gln Leu Pro (SEQ ID NO:47), [Bakke et al., Cell 63:707-716 (1990)] for the endosomal compartment. See, Letourneur et al., Cell 69:1183 (1992) for targeting liposomes. Myristolation sequences can be used to direct the antibody to the plasma membrane. In addition, as shown in Table 2 below, myristoylation sequences can be used to direct the antibodies to different subcellular locations such as the nuclear region. Localization sequences may also be used to direct antibodies to organelles, such as the mitochondria and the Golgi apparatus. The sequence Met Leu Phe Asn Leu Arg Xaa Xaa Leu Asn Asn Ala Ala Phe Arg His Gly His Asn Phe Met Val Arg Asn Phe Arg Cys Gly Gln Pro Leu Xaa (SEQ ID NO:48) can be used to direct the antibody to the mitochondrial matrix, (Pugsley, supra). See, Tang et al., J. Biol. Chem. 267:10122 (1992), for localization of proteins to the Golgi apparatus.

TABLE 2

| AMINO- | SUB- | | |
|---|---|---|---|
| TERMINAL | CELLULAR | | |
| SEQUENCE | LOCATION** | PROTEIN | REFERENCE |
| GCVCSSNP | PM | p56^{USTRATCK} | Marchildon, et al. |
| (SEQ ID | | | Proc. Natl. Acad. Sci. |
| NO:49) | | | USA 81: 7679-7682 |
| | | | (1984) |
| | | | Voronova, et al. Mol. |
| | | | Cell. Biol. 4: |
| | | | 2705-2713 (1984) |
| GQTVTTPL | PM | Mul.V gag | Henderson, et al., |
| (SEQ ID | | | Proc. Natl. Acad. Sci. |
| NO:50) | | | USA 80: 339-343 |
| | | | (1987) |
| GQELSQHE | PM | M-PMV gag | Rhee, et al., J. Virol. |
| (SEQ ID | | | 61: 1045-1053 (1987) |
| NO:51) | | | Schultz, et al., J. |
| | | | Virol. 46: 355-361 |
| | | | (1983) |
| GNSPSYNP | PM | BLV gag | Schultz, et al., J. |
| (SEQ ID | | | Virol. 133: 431-437 |
| NO:52) | | | (1984) |
| GVSGSKGQ | PM | MMTV gag | Schultz et al., supra |
| (SEQ ID | | | |
| NO:53) | | | |
| GQTITTPL | PM | FCL.V gag | Schultz et al., supra |
| (SEQ ID | | | |
| NO:54) | | | |
| GQTLTTPL | PM | BaEV gag | Schultz et al., supra |
| (SEQ ID | | | |
| NO:55) | | | |
| GQIFSRSA | PM | HTLV-I gag | Ootsuyama, et al., Jpn |
| (SEQ ID | | | J. Cancer Res. 76: |
| NO:56) | | | 1132-1135 (1985) |
| GQIHGLSP | PM | HTLV-II gag | Ootsuyama, et al., |
| (SEQ ID | | | supra |
| NO:57) | | | |
| GARASVLS | PM | HIV | Ratner et al., Nature |
| (SEQ ID | | (HTLV-III) | 313: 277-284 (1985) |
| NO:58) | | gag | |
| GCTLSAEE | PM | bovine brain | Schultz et al., |
| (SEQ ID | | G₀ α-subunit | Biochem. |
| NO:59) | | | Biophys. Res. |
| | | | Commun. 146: |
| | | | 1234-1239 (1987) |
| GQNLSTSN | ER | Hepatitis B | Persing et al., J. Virol. |
| (SEQ ID | | Virus pre-S1 | 61: 1672-1677 (1987) |
| NO:60) | | | |
| GAALTILV | N | Polyoma | Streuli et al., Nature |
| (SEQ ID | | Virus VP2 | 326: 619-622 (1987) |
| NO:61) | | | |
| GAALTLLG | N | SV40 | Streuli et al., supra |
| (SEQ ID | | Virus VP2 | |
| NO:62) | | | |
| GAQVSSQK | S,ER | Poliovirus | Chow et al., Nature |
| (SEQ ID | | VP4 | 327: 482-486 (1987) |
| NO:63) | | | Paul et al., Proc. Natl. |
| | | | Acad. Sci. USA 84: |
| | | | 7827-7831 (1987) |
| GAQLSRNT | S,ER | Bovine | Paul et al., supra |
| (SEQ ID | | Enterovirus | |
| NO:64) | | VP4 | |
| GNAAAAKK | G,S,N,C | cAMP- | Carr et al., Proc. Natl. |
| (SEQ ID | | dependent | Acad. Sci. USA 79: |
| NO:65) | | kinase | 6128-6131 (1982) |
| GNEASYPL | S,C | calcincurin B | Aitken et al. FEBS |
| (SEQ ID | | | Lett. 150: 314-318 |
| NO:66) | | | (1982) |
| GSSKSKPK | PM,C | P60^{SFC} | Schultz et al., Science |
| (SEQ ID | | | 227: 427-429 (1985) |
| NO:67) | | | |

**Abbreviations are PM, plasma membranes, G, Golgi; N, Nuclear; C, Cytoskeleton; S, cytoplasm (soluble); M, membrane.
Additional heterologous sequences may be found in Example 1 and Persic, et al., Gene 187: 1-8 (1997).

In certain embodiments, the heterologous polypeptide fusion partner is a stability sequence to confer stability to the candidate regulator polypeptide or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)ₙ GGPP (SEQ ID NO:69), where X is any amino acid and n is an integer of at least four.

In one embodiment, the heterologous polypeptide fusion partner is a dimerization sequence. A dimerization sequence allows the non-covalent association of one random candidate peptide to another candidate random peptide, with sufficient affinity to remain associated under normal physiological conditions. This effectively allows small libraries of candidate regulator polypeptides (for example, 10⁴) to become large libraries if two peptides per cell are generated which then dimerize, to form an effective library of 10⁸ (10⁴ times 10⁴). It also allows the formation of longer random peptides, if needed, or more structurally complex random peptide molecules. The dimers may be homo- or heterodimers.

Dimerization sequences may be a single sequence that self-aggregates, or two sequences, each of which is generated in a different viral construct. That is, nucleic acids encoding both a first random peptide with dimerization sequence 1, and a second random peptide with dimerization sequence 2, such that upon introduction into a cell and expression of the nucleic acid, dimerization sequence 1 associates with dimerization sequence 2 to form a new random peptide structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

The heterologous polypeptide fusion partners may be placed anywhere (i.e. N-terminal, C-terminal, internal) in the structure as the biology and activity permits.

In certain embodiments, a heterologous polypeptide fusion partner includes a linker or tethering sequence. Linker sequences between various targeting sequences (for example, membrane targeting sequences) and the other components of the constructs (such as the candidate peptides or molecular scaffold polypeptides) may be desirable to allow the candidate regulator polypeptides to interact with potential targets unhindered. For example, useful linkers include glycine-serine polymers (including, for example, (GS)ₙ (SEQ ID NO:70), (GSGGS)ₙ (SEQ ID NO:71) and (GGGS)ₙ (SEQ ID NO:72), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In addition, fusion partners, as well as molecular scaffolds as described herein, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

In a preferred embodiment, a regulator polypeptide comprising a candidate peptide linked to a molecular scaffold is added at a cloning site, e.g., Rp, in Table 1 above. Alternatively, in certain embodiments, no molecular scaffold is used in the regulator polypeptide, giving a “free” or “non-constrained” peptide or expression product.

Based on the above description of regulator polypeptides, examples include the following:

a) intracellular, membrane-anchored, linked (i.e. tethered) free peptide: MRPLAGGEHTMASPLTRFLSLNLLLLGESIILGSGPQRPEDCRPRGSVK GTGLDFA CDIYIWAPLAGICVALLLSLIITLICYHSR-GSGGSGSGGSGSGGSGSGGSGSGGSGGG-(X)ₙ-GGPP (SEQ ID NO:73), with the secretion signal from murine CD8 in bold, the transmembrane region of CD8 in underline, and the linker, to provide flexibility (glycine) and solubility (serine) in italics. (X)ₙ represents the random peptide, where n is an integer greater than about six. One embodiment utilizing this structure utilizes biased peptides, as described below, for example using biased SH-3 domain-binding peptide libraries in the non-constrained peptide structures, since a number of surface receptor signaling systems employ SH-3 domains as part of the signaling apparatus.

b) intracellular, membrane-anchored, linked coiled coil: MRPLAGGEHTMASPL TRFLSLNLLLLGESIILGSG PQRPEDCRPRGSVKGTGLDFA CDIYIWAPLAGICVALLLSL IITLICYHSRGSGGSGSGGSGSGGSGSGGSGSGGSGG GCAALESEVSALESEVASLESEVAAL-(X)ₙ-LAAVKSKL SAVKSKLASVKSKLAACGPP (SEQ ID NO:74), with the coiled-coil structure shown in underlined italics.

c) surface-tethered extracellular, non-constrained: MRPLAGGEHTMASPLTRFLSLNLLLLGESHLGSGGG-(X)ₙ-GGSGGSGSGGSGSGGSGSGGSGSGGSGGGPQRPEDCRPRGSVKGTGL DFACDI YIWAPLAGICVALLLSLIITLICYHSRGGPP (SEQ ID NO:75).

d) surface-tethered, extracellular constrained: MRPLAGGEHT MASPLTRFLSLNLLLLGESIILGSGGGCAALESEVSALESEVASLESEVA AL-(X)ₙ-LAAVKSKLSAVKSKLASVKSKLAAC GGSGGSGSGGSGSGG SGSGGSGSGGSGGG PQRPEDCRPRGSVKGTGLD FACDIYIWAPLA GICVALLLSLHTLICYHSRGGPP (SEQ ID NO:76).

e) secreted, non-constrained: MRPLAGGEHTMASPLTRFLSLNLL LLGESIILGSGGG-(X)ₙ-GGPP (SEQ ID NO:77).

f) secreted, constrained: MRPLAGGEHTMASPLTRFLSLNLLLL GESIILGSGG GAALESEVSALESEVASLESEVAAL-(X)ₙ-LAA VKSKLSAVKSKLASVKSKLAACGPP (SEQ ID NO:78).

The candidate regulator polypeptides as described above are encoded by candidate polynucleotides or candidate nucleic acids. By “candidate polynucleotides” or “candidate nucleic acids” herein is meant a polynucleotide or nucleic acid, generally RNA when retroviral vectors are used and generally DNA when nonretroviral vectors such as poxviruses are used, which can be expressed to form candidate regulator molecules; that is, the candidate polynucleotides or nucleic acids encode the candidate regulator polypeptides and the fusion partners, if present, or encode the candidate regulator RNAs such as candidate U1 SnRNAs. In addition, the candidate polynucleotides or nucleic acids will also generally contain enough extra sequence to effect translation or transcription, as necessary. For a regulator polypeptide library, the candidate polynucleotide or nucleic acid generally contains cloning sites which are placed to allow in frame expression of the randomized peptides, molecular scaffolds, and any fusion partners, if present. For example, when molecular scaffolds are used, the molecular scaffold will generally contain the initiating ATG, as a part of the parent vector. Suitable and preferred poxvirus vectors are disclosed herein.

Generally, the candidate polynucleotides or nucleic acids are expressed within the cells to produce expression products of the candidate polynucleotides or nucleic acids. As outlined above, the expression products include translation products, i.e. peptides, or transcription products, i.e., RNA.

The candidate peptide or candidate RNA fragment portions of candidate regulator molecules are randomized, either fully randomized or they are biased in their randomization, e.g. in nucleotide/residue frequency generally or per position. By “randomized” or grammatical equivalents herein is meant that each candidate peptide or RNA fragment consists of essentially random amino acids and nucleotides, respectively. As is more fully described below, the candidate nucleic acids which give rise to the candidate peptides or candidate RNA fragments are chemically synthesized, and thus may incorporate any nucleotide at any position. Thus, when the candidate nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized candidate nucleic acids.

The library should provide a sufficiently structurally diverse population of randomized expression products to effect a probabilistically sufficient range of cellular responses to provide one or more cells exhibiting a desired response. Accordingly, an interaction library must be large enough so that at least one of its members will have a structure that gives it affinity for some molecule, protein, or other factor whose activity is necessary for completion of the signaling pathway of a target transcriptional regulatory region which is naturally induced in a target cellular process. Although it may be difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, for example, has the potential to code for 20⁷ (10⁹) to 20²⁰. Thus, with libraries of 10⁷ to 10⁸ per ml of viral particles the present methods allow a “working” subset of a theoretically complete interaction library for 7 amino acids, and a subset of shapes for the 20²⁰ library. Thus, in a preferred embodiment, at least 10³,at least 10⁴,at least 10⁵, at least 10⁶, at least 10⁷, at least 10⁸, at least 10⁹, or at least 10¹⁰ different expression products are simultaneously analyzed in the subject methods. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is an unacceptable high likelihood that 46.7% of the peptides will prematurely terminate. For free peptide structures this is perhaps not a problem. But for larger structures, such as those envisioned here, such termination will lead to sterile peptide expression. To alleviate this, random residues are encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely. For candidate nucleic acids which are not designed to result in peptide expression products, this is not necessary.

In one embodiment, the library is fully randomized, with no sequence preferences or constants at any position. In another embodiment, the library is biased. That is, some positions within the sequence are either held constant, or are selected from a limited number of possibilities. For example, in one embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In another embodiment, the bias is towards molecules or nucleic acids that interact with known classes of molecules. For example, when the candidate regulator molecule is a polypeptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as pseudosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate regulator polypeptides as well.

Thus, a number of molecules or protein domains are suitable as starting points for the generation of biased randomized candidate regulator polypeptides. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

Where the desired regulator molecule is a nucleic acid, at least 5, at least 10, at least 12, at least 15, or at least 21 nucleotide positions need to be randomized, with more randomized nucleotides being preferable if the randomization is less than perfect. Similarly, at least 2, at least 3, at least 4, at least 5, at least 6, or at least 7 amino acid positions need to be randomized; again, more are preferable if the randomization is less than perfect.

In one embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases:

1. XXXPPXPXX (SEQ ID NO:79), wherein X is a randomized residue;

2. Met Gly aa11 aa10 aa9 aa8 aa7 Arg Pro Leu Pro Pro hyd Pro hyd hyd Gly Gly Pro Pro STOP (SEQ ID NO:81) encoded by the following nucleotide sequence: atg ggc nnk nnk nnk nnk nnk aga cct ctg cct cca sbk ggg sbk sbk gga ggc cca cct TAA. (SEQ ID NO:80).

In this embodiment, the N-terminus flanking region is suggested to have the greatest effects on binding affinity and is therefore entirely randomized. “Hyd” indicates a bias toward a hydrophobic residue, e.g.,—ala, val, met, leu, ile, phe, tyr, trp, pro, cys. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

Candidate polynucleotides are introduced into eukaryotic host cells to select and/or screen for regulator polypeptides capable of altering the phenotype of a cell. By “introduced into” or grammatical equivalents herein is meant that the polynucleotides enter the cells in a manner suitable for subsequent expression. The method of introduction is largely dictated by the host cell type, discussed below. Exemplary methods include CaPO₄ precipitation, liposome fusion, lipofectin™, electroporation, viral infection, etc. A preferred method of introduction is through use of a poxvirus vector library constructed through trimolecular recombination. The candidate polynucleotides may stably integrate into the genome of the host cell (for example, with retroviral introduction), or may exist either transiently or stably in the cytoplasm (i.e. through the use of poxvirus such as vaccinia virus, traditional plasmids, utilizing standard regulatory sequences, selection markers, etc.). As many pharmaceutically important screens require human or model mammalian cell targets, vaccinia virus vectors or other mammalian viral vectors capable of transfecting such targets are preferred.

In a preferred embodiment, the candidate polynucleotides are introduced as part of a viral particle which infects the cells. Generally, infection of the cells is straightforward. Infection may be performed using the infection-enhancing reagent polybrene, which is a polycation that facilitates viral binding to the target cell. Infection can be optimized such that each cell generally expresses a single construct, using the ratio of virus particles to number of cells. Infection follows a Poisson distribution.

In one embodiment, the candidate nucleic acids are introduced into the cells using retroviral vectors. Currently, the most efficient gene transfer methodologies harness the capacity of engineered viruses, such as retroviruses, to bypass natural cellular barriers to exogenous nucleic acid uptake. The use of recombinant retroviruses was pioneered by Richard Mulligan and David Baltimore with the Psi-2 lines and analogous retrovirus packaging systems, based on NIH 3T3 cells (see Mann et al., Cell 33:153-159 (1993), hereby incorporated by reference). Such helper-defective packaging lines are capable of producing all the necessary trans proteins -gag, pol, and env- that are required for packaging, processing, reverse transcription, and integration of recombinant genomes. Those RNA molecules that have in cis the .psi. packaging signal are packaged into maturing virions. Retroviruses are preferred for a number of reasons. First, their derivation is easy. Second, unlike Adenovirus-mediated gene delivery, expression from retroviruses is long-term (adenoviruses do not integrate). Adeno-associated viruses have limited space for genes and regulatory units and there is some controversy as to their ability to integrate. Retroviruses therefore offer the best current compromise in terms of long-term expression, genomic flexibility, and stable integration, among other features. The main advantage of retroviruses is that their integration into the host genome allows for their stable transmission through cell division. This ensures that in cell types which undergo multiple independent maturation steps, such as hematopoietic cell progression, the retrovirus construct will remain resident and continue to express.

A particularly well suited retroviral transfection system is described in Mann et al., supra: Pear et al., Proc. Natl. Acad. Sci. USA 90(18):8392-6 (1993); Kitamura et al., Proc. Natl. Acad. Sci. USA 92:9146-9150 (1995); Kinsella et al., Human Gene Therapy 7:1405-1413; Hofmann et al., Proc. Natl. Acad. Sci. USA 93:5185-5190; Choate et al., Human Gene Therapy 7:2247 (1996); and WO 94/19478; and references cited therein, all of which are incorporated by reference.

In one embodiment of the invention, the library is generated in a viral DNA genome backbone, for example, a poxvirus genome. Suitable and preferred methods for preparing poxvirus libraries are disclosed in the Examples herein.

In an alternative method, standard oligonucleotide synthesis is done to generate the random portion of the candidate regulator polypeptide, using techniques well known in the art (see Eckstein, Oligonucleotides and Analogues, A Practical Approach, IRL Press at Oxford University Press, 1991). Libraries may be commercially purchased. Libraries with up to 10⁹ unique sequences can be readily generated in such DNA viral backbones. After generation of the DNA library, the library is cloned into a first primer. The first primer serves as a “cassette”, which is inserted into the viral construct. The first primer generally contains a number of elements, including for example, the required regulatory sequences (e.g. translation, transcription, promoters, etc), fusion partners, restriction endonuclease (cloning and subcloning) sites, stop codons (preferably in all three frames), regions of complementarity for second strand priming (preferably at the end of the stop codon region as minor deletions or insertions may occur in the random region), etc.

A second primer is then added, which generally consists of some or all of the complementarity region to prime the first primer and optional necessary sequences for a second unique restriction site for subcloning. DNA polymerase is added to make double-stranded oligonucleotides. The double-stranded oligonucletides are cleaved with the appropriate subcloning restriction endonucleases and subcloned into the target viral vectors, described below.

The viruses may include inducible and constitutive promoters. For example, there are situations wherein it is necessary to induce peptide or mRNA expression only during certain phases of the selection process. A large number of both inducible and constitutive promoters are known.

In this manner the primers create a library of fragments, each containing a different random nucleotide sequence that may encode a different peptide. The ligation products are then transformed into bacteria, such as E. coli, and DNA is prepared from the resulting library, as is generally outlined in Kitamura Proc.

Natl. Acad. Sci. USA 92:9146-9150 (1995), hereby expressly incorporated by reference.

Delivery of the library DNA into a viral packaging system results in conversion to infectious virus.

The candidate nucleic acids, as part of the viral construct, are introduced into the cells to screen for regulator molecules capable of modifying the phenotype of a cell through induction or inhibition of activation of a target transcriptional regulatory region.

As will be appreciated by those in the art, the type of host cells used in the present invention can vary widely. Generally, any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a selection and/or screen will be set up in the host cells such that individual host cells exhibit a modified phenotype in the presence of a desired regulator molecule. As is more fully described below, cell types implicated in a wide variety of disease conditions and differentiation states are particularly useful, so long as a suitable selection and/or screen may be designed to allow the identification of individual cells that exhibit a modified phenotype as a consequence of the presence of a desired regulator molecule within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell) , mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. Additional host cell lines may be identified in the catalog the America Type Culture Collection (ATCC), available at their web site<http://www.atcc.org/>, and hereby expressly incorporated by reference. Additional host cell types are given in the Examples herein.

In one embodiment, the host cells may be genetically engineered, that is, contain erogenous nucleic acid, for example, to contain target molecules, reporter constructs, or suicide proteins.

In one embodiment, a first population of host cells into which the library is introduced is subjected to selection and/or is screened. That is, the host cells into which the candidate polynucleotidess are introduced are subjected to selection and/or are screened for a modified phenotype. Thus, in this embodiment, the effect of the regulator moledule is seen in the same cells in which it is made; i.e. an autocrine effect.

By a “plurality of cells” or a “population of host cells” herein is meant roughly from about 10³ cells to 10⁸ or 10⁹, with from 10⁶ to 10⁸ being preferred. This plurality of cells comprises a cellular library, wherein generally each cell within the library contains a member of the viral molecular library, i.e., a different candidate polynucleotide, although as will be appreciated by those in the art, some cells within the library may not contain a viral vector, and some may contain more than one. When methods other than viral infection are used to introduce the candidate polynucleotides into a plurality of cells, the distribution of candidate polynucleotides within the individual cell members of the cellular library may vary widely, as it is generally difficult to control the number of nucleic acids which enter a cell during electroporation, etc.

In certain embodiments, the candidate polynucleotides are introduced into a first plurality of cells, and the effect of the candidate regulator molecules, e.g., regulator polypeptides or regulator U1 SnRNAs, is screened in a second or third plurality of cells, different from the first plurality of cells, i.e., generally a different cell type. That is, the effect of the regulator molecules is due to an extracellular effect on a second cell; i.e., an endocrine or paracrine effect. This is done using standard techniques. The first plurality of cells may be grown in or on one media, and the media is allowed to touch a second plurality of cells, and the effect measured. Alternatively, there may be direct contact between the cells. Thus, “contacting” is functional contact, and includes both direct and indirect. In this embodiment, the first plurality of cells may or may not be screened.

If necessary, the cells are treated to conditions suitable for the expression of the candidate polynucleotidess (for example, when inducible promoters are used), to produce the candidate expression products, either translation or transcription products.

Thus, the methods of the present invention comprise introducing a molecular library of randomized candidate polynucleotides into a plurality of host cells, a cellular library. Each of the polynucleotides comprises a different, generally randomized, nucleotide sequence. The plurality of cells is then subjected to selection and/or screening, as is more fully outlined below, for a cell exhibiting a predetermined modified phenotype. The modified phenotype is due to the presence of a regulator molecule.

By “altered phenotype,” “modified phenotype,” “changed physiology” or other grammatical equivalents herein is meant that the phenotype of the cell is modified in some way, preferably in some detectable and/or measurable way. As will be appreciated in the art, a strength of the present invention is the wide variety of cell types and potential phenotypic changes which may be tested using the present methods, in particular. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptibility, latency, adhesion, and uptake of viruses and bacterial pathogens; in the embodiments where poxvirus vectors are concerned, the phenotypic change can include host cell death. By “capable of modifying the phenotype” herein is meant that the regulator polypeptide can modify the phenotype of the cell in some detectable and/or measurable way, either directly or indirectly, through inducing or inhibiting activation of a target transcriptional regulatory region.

The modified phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the modified phenotype is detected in the cell in which the polynucleotide encoding the desired regulator molecule was introduced; in other embodiments, the modified phenotype is detected in a second cell which is responding to some molecular signal from the first cell.

An modified phenotype of a host cell indicates the presence of a desired regulator molecule. By “transdominant” herein is meant that the regulator molecule indirectly causes the modified phenotype by acting on a second molecule, which leads to a modified phenotype. That is, a transdominant expression product has an effect that is not in cis, i.e., a trans event as defined in genetic terms or biochemical terms. A transdominant effect is a distinguishable effect by a molecular entity (i.e., the encoded peptide or RNA) upon some separate and distinguishable target; that is, not an effect upon the encoded entity itself. As such, transdominant effects include many well-known effects by pharmacologic agents upon target molecules or pathways in cells or physiologic systems; for instance, the β-lactam antibiotics have a transdominant effect upon peptidoglycan synthesis in bacterial cells by binding to penicillin binding proteins and disrupting their functions. An exemplary transdominant effect by a peptide is the ability to inhibit NF-KB signaling by binding to IKB-a at a region critical for its function, such that in the presence of sufficient amounts of the peptide (or molecular entity), the signaling pathways that normally lead to the activation of NF-KB through phosphorylation and/or degradation of IKB-α are inhibited from acting at IKB-a because of the binding of the peptide or molecular entity. In another instance, signaling pathways that are normally activated to secrete IgE are inhibited in the presence of peptide. Or, signaling pathways in adipose tissue cells, normally quiescent, are activated to metabolize fat. Or, in the presence of a peptide, intracellular mechanisms for the replication of certain viruses, such as HIV-I, or Herpes viridae family members, or Respiratory Syncytial Virus, for example, are inhibited.

A transdominant effect upon a protein or molecular pathway is clearly distinguishable from randomization, change, or mutation of a sequence within a protein or molecule of known or unknown function to enhance or diminish a biochemical ability that protein or molecule already manifests. For instance, a protein that enzymatically cleaves β-lactam antibiotics, a β-lactamase, could be enhanced or diminished in its activity by mutating sequences internal to its structure that enhance or diminish the ability of this enzyme to act upon and cleave β-lactam antibiotics. This would be called a cis mutation to the protein. The effect of this protein upon β-lactam antibiotics is an activity the protein already manifests, to a distinguishable degree. Similarly, a mutation in the leader sequence that enhanced the export of this protein to the extracellular spaces wherein it might encounter β-lactam molecules more readily, or a mutation within the sequence that enhance the stability of the protein, would be termed cis mutations in the protein. For comparison, a transdominant effector of this protein would include an agent, independent of the β-lactamase, that bound to the β-lactamase in such a way that it enhanced or diminished the function of the β-lactamase lactamase by virtue of its binding to β-lactamase.

In general, cis-effects are effects within molecules wherein elements that are interacting are covalently joined to each other although these elements might individually manifest themselves as separable domains. Trans-effects (transdominant in that under some cellular conditions the desired effect is manifested) are those effects between distinct molecular entities, such that molecular entity A, not covalently linked to molecular entity B, binds to or otherwise has an effect upon the activities of entity B. As such, most known pharmacological agents are transdominant effectors.

In a preferred embodiment, once a host cell with an modified phenotype is detected, the host cell is recovered and/or isolated from the plurality which do not have modified phenotype. This may be done in any number of ways, as is known in the art, and will in some instances depend on the selection scheme, assay or screen. Suitable recover or isolation techniques include, but are not limited to, apoptosis, nonadherence of a normally adherent host cell, FACS, lysis selection using complement, cell cloning, scanning by Fluorimager, expression of a “survival” protein, expression of a “suicide” protein, induced expression of a cell surface protein or other molecule that can be rendered fluorescent or taggable for physical isolation; expression of an enzyme that changes a non-flourescent molecule to a fluoroscent one; overgrowth against a background of no or slow growth; death of cells and isolation of DNA or other cell vitality indicator dyes, etc.

In a preferred embodiment, polynucleotides encoding desired regulator molecules are “isolated” from recovered host cells, i.e., they are substantially removed from their native environment and are largely separated from polynucleotides in the library which do not encode regulator molecules of interest. This may be done in a number of ways. In a preferred embodiment, primers complementary to DNA regions common to the viral constructs, or to specific components of the library such as a rescue sequence, defined above, are used to “rescue” the unique random sequence. Alternatively, the regulator polypeptide is isolated using a rescue sequence. Thus, for example, rescue sequences comprising epitope tags or purification sequences may be used to pull out the regulator polypeptide, using immunoprecipitation or affinity columns. In some instances, as is outlined below, this may also pull out the primary target molecule, if there is a sufficiently strong binding interaction between the regulator polypeptide and the target molecule. Alternatively, the peptide may be detected using mass spectroscopy.

Once rescued, the sequence of the regulator molecule is determined. This information can then be used in a number of ways.

In a certain embodiments, the regulator molecule is resynthesized and reintroduced into the target cells, to verify the effect. This may be done using viral vectors, e.g., poxviruses or retroviruses, or alternatively using fusions to the HIV-1 Tat protein, and analogs and related proteins, which allows very high uptake into target cells. See for example, Fawell et al., Proc. Natl. Acad. Sci. USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In a preferred embodiment, the sequence of a regulator molecule is used to generate more candidate regulator molecules. For example, the sequence of the randomized regions of the regulator molecule may be the basis of a second round of (biased) randomization, to develop regulator molecules with increased or altered activities. Alternatively, the second round of randomization may change the affinity of the regulator polypeptide. Furthermore, it may be desirable to put the identified random region of the regulator molecule into other molecular scaffolds, or to alter the sequence of the constant region of the molecular scaffold, to alter the conformation/shape of the regulator molecule. It may also be desirable to “walk” around a potential binding site, in a manner similar to the mutagenesis of a binding pocket, by keeping one end of the ligand region constant and randomizing the other end to shift the binding of the peptide around.

In certain embodiments, either the regulator molecule or the polynucleotide encoding it is used to identify target molecules, i.e., the molecules, e.g., regulatory regions, with which the regulator molecule interacts. As will be appreciated by those in the art, there may be primary target molecules, to which the regulator polypeptide binds or acts upon directly, and there may be secondary target molecules, which are part of the signalling pathway affected by the regulator polypeptide; these might be termed “validated targets”.

In a preferred embodiment, the regulator molecule is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a cDNA expression library made from mRNA of the target cell type. Or, peptides can be used as “bait” in either yeast or mammalian two or three hybrid systems. Such interaction cloning approaches have been very useful to isolate DNA-binding proteins and other interacting protein components. The peptide(s) can be combined with other pharmacologic activators to study the epistatic relationships of signal transduction pathways in question. It is also possible to synthetically prepare isolated labeled regulator molecules and use tem to screen a cDNA library expressed in bacteriophage or vaccinia virus library for those cDNAs which bind the regulator molecule. Furthermore, it is also possible that one could use cDNA cloning via viral libraries to “complement” the effect induced by the regulator molecule. In such a strategy, the regulator molecule would be required to be stochiometrically titrating away some important factor for a specific signaling pathway. If this molecule or activity is replenished by over-expression of a cDNA from within a cDNA library, then one can clone the target. Similarly, cDNAs cloned by any of the above library systems can be reintroduced to mammalian cells in this manner to confirm that they act to complement function in the system the peptide acts upon.

Once primary target molecules have been identified, secondary target molecules may be identified in the same manner, using the primary target as the “bait”. In this manner, signalling pathways may be elucidated. Similarly, regulator molecules specific for secondary target molecules may also be discovered, to allow a number of regulator molecules to act on a single pathway, for example for combination therapies.

The screening and/or selection methods of the present invention may be useful to screen a large number of cell types under a wide variety of conditions. In certain embodiments, the host cells are cells that are involved in disease states, and they are tested or screened under conditions that normally result in undesirable consequences on the cells. When a suitable regulator molecule is found, the undesirable effect may be reduced or eliminated. Alternatively, normally desirable consequences may be reduced or eliminated, with an eye towards elucidating the cellular mechanisms associated with the disease state or signalling pathway.

In certain embodiments, the present methods are useful in cancer applications. The ability to rapidly and specifically kill tumor cells is a cornerstone of cancer chemotherapy. In general, using the methods of the present invention, random libraries of candidate regulator molecules can be introduced into any tumor cell (primary or cultured), and desired regulator molecules identified which by themselves induce apoptosis, cell death, loss of cell division or decreased cell growth. This may be done de novo, or by biased randomization toward known peptide agents, such as angiostatin, which inhibits blood vessel wall growth. Alternatively, the methods of the present invention can be combined with other cancer therapeutics (e.g. drugs or radiation) to sensitize the cells and thus induce rapid and specific apoptosis, cell death, loss of cell division or decreased cell growth after exposure to a secondary agent. Similarly, the present methods may be used in conjunction with known cancer therapeutics to screen for agonists to make the therapeutic more effective or less toxic. This is particularly preferred when the chemotherapeutic is very expensive to produce such as taxon.

Known oncogenes such as v-AbI, v-Src, v-Ras, and others, induce a transformed phenotype leading to abnormal cell growth when transfected into certain cells. This is also a major problem with micro-metastases. Thus, in a preferred embodiment, non-transformed cells can be transfected with these oncogenes, and then random libraries of candidate regulator molecules introduced into these cells, to select and/or screen for regulator molecules which reverse or correct the transformed state. One of the signal features of oncogene transformation of cells is the loss of contact inhibition and the ability to grow in soft-agar. When transforming viruses are constructed containing v-Abl, v-Src, or v-Ras in viral vectors, infected into target 3T3 cells, and subjected to puromycin selection, all of the 3T3 cells hyper-transform and detach from the plate. The cells may be removed by washing with fresh medium. This can serve as the basis of a screen, since cells which express a desired regulator molecule will remain attached to the plate and form colonies.

Similarly, the growth and/or spread of certain tumor types is enhanced by stimulatory responses from growth factors and cytokines (PDGF, EGF, Heregulin, and others) which bind to receptors on the surfaces of specific tumors. In one embodiment, the methods of the invention are used to inhibit or stop tumor growth and/or spread, by finding regulator molecules capable of blocking the ability of the growth factor or cytokine to stimulate the tumor cell. The introduction of random libraries of candidate regulator molecules into specific tumor cells with the addition of the growth factor or cytokine, followed by selection and/or screening of regulator molecules which block the binding, signaling, phenotypic and/or functional responses of these tumor cells to the growth factor or cytokine in question.

Similarly, the spread of cancer cells (invasion and metastasis) is a significant problem limiting the success of cancer therapies. The ability to inhibit the invasion and/or migration of specific tumor cells would be a significant advance in the therapy of cancer. Tumor cells known to have a high metastatic potential (for example, melanoma, lung cell carcinoma, breast and ovarian carcinoma) can have random libraries of candidate regulator molecules introduced into them, wit desired regulator molecules being selected which in a migration or invasion assay, inhibit the migration and/or invasion of specific tumor cells. Particular applications for inhibition of the metastatic phenotype, which could allow a more specific inhibition of metastasis, include the metastasis suppressor gene NM23, which codes for a dinucleoside diphosphate kinase. Thus intracellular peptide activators of this gene could block metastasis, and a screen for its upregulation (by fusing it to a reporter gene) would be of interest. Many oncogenes also enhance metastasis. Peptides which inactivate or counteract mutated RAS oncogenes, v-MOS, v-RAF, A-RAF, v-SRC, v-FES, and v-FMS would also act as anti-metastatics. Peptides which act intracellularly to block the release of combinations of proteases required for invasion, such as the matrix metalloproteases and urokinase, could also be effective antimetastatics.

In another embodiment, the random libraries of candidate regulator molecules of the present invention are introduced into tumor cells known to have inactivated tumor suppressor genes, and successful reversal by either reactivation or compensation of the knockout would be screened by restoration of the normal phenotype. A major example is the reversal of p53-inactivating mutations, which are present in 50% or more of all cancers. Since p53's actions are complex and involve its action as a transcription factor, there are probably numerous potential ways a peptide or small molecule derived from a peptide could reverse the mutation. One example would be upregulation of the immediately downstream cyclin-dependent kinase p21CIP1/WAF1. To be useful such reversal would have to work for many of the different known p53 mutations. This is currently being approached by gene therapy; one or more small molecules which do this might be preferable.

Another example involves screening for and/or selection of regulator molecules which restore the constitutive function of the brca-1 or brca-2 genes, and other tumor suppressor genes important in breast cancer such as the adenomatous polyposis coli gene (APC) and the Drosophila discs-large gene (Dlg), which are components of cell—cell junctions. Mutations of brca-1 are important in hereditary ovarian and breast cancers, and constitute an additional application of the present invention.

In another embodiment, the methods of the present invention are used to create novel cell lines from cancers from patients. A virally delivered short regulator molecule which inhibits the final common pathway of programmed cell death should allow for short- and possibly long-term cell lines to be established. Conditions of in vitro culture and infection of human leukemia cells will be established. There is a real need for methods which allow the maintenance of certain tumor cells in culture long enough to allow for physiological and pharmacological studies. Currently, some human cell lines have been established by the use of transforming agents such as Epstein-Barr virus that considerably alters the existing physiology of the cell. On occasion, cells will grow on their own in culture but this is a random event. Programmed cell death (apoptosis) occurs via complex signaling pathways within cells that ultimately activate a final common pathway producing characteristic changes in the cell leading to a non-inflammatory destruction of the cell. It is well known that tumor cells have a high apoptotic index, or propensity to enter apoptosis in vivo. When cells are placed in culture, the in vivo stimuli for malignant cell growth are removed and cells readily undergo apoptosis. The objective would be to develop the technology to establish cell lines from any number of primary tumor cells, for example primary human leukemia cells, in a reproducible manner without altering the native configuration of the signaling pathways in these cells. By introducing nucleic acids encoding regulator molecules which inhibit apoptosis, increased cell survival in vitro, and hence the opportunity to study signalling transduction pathways in primary human tumor cells, is accomplished. In addition, these methods may be used for culturing primary cells, i.e., non-tumor cells.

In yet another embodiment, the present methods are useful in cardiovascular applications. For example, cardiomyocytes may be screened for the prevention of cell damage or death in the presence of normally injurious conditions, including, but not limited to, the presence of toxic drugs (particularly chemotherapeutic drugs), for example, to prevent heart failure following treatment with adriamycin; anoxia, for example in the setting of coronary artery occlusion; and autoimmune cellular damage by attack from activated lymphoid cells (for example as seen in post viral myocarditis and lupus). Candidate regulator molecules are inserted into cardiomyocytes, the cells are subjected to the insult, and regulator molecules are selected that prevent any or all of: apoptosis; membrane depolarization (i.e. decrease arrythmogenic potential of insult); cell swelling; or leakage of specific intracellular ions, second messengers and activating molecules (for example, arachidonic acid and/or lysophosphatidic acid).

In a similar embodiment, the present methods are used to screen for diminished arrhythmia potential in cardiomyocytes. The screens comprise the introduction of the candidate nucleic acids encoding candidate regulator molecules, followed by the application of arrythmogenic insults, with screening for regulator molecules that block specific depolarization of cell membrane. This may be detected using patch clamps, or via fluorescence techniques). Similarly, channel activity (for example, potassium and chloride channels) in cardiomyocytes could be regulated using the present methods in order to enhance contractility and prevent or diminish arrhythmia.

In a further embodiment, the present methods are used to screen for enhanced contractile properties of cardiomyocytes and diminish heart failure potential. The introduction of the libraries encoding candidate regulator molecules of the invention followed by measuring the rate of change of myosin polymerization/depolymerization using fluorescent techniques can be done. Regulator molecules which increase the rate of change of this phenomenon can result in a greater contractile response of the entire myocardium, similar to the effect seen with digitalis.

In an additional embodiment, the present methods are useful to identify regulator molecules that will regulate the intracellular and sarcolemmal calcium cycling in cardiomyocytes in order to prevent arrhythmias. Regulator molecules are identified that regulate sodium-calcium exchange, sodium proton pump function, and regulation of calcium-ATPase activity.

In another embodiment, the present methods are useful to identify regulator molecules that diminish embolic phenomena in arteries and arterioles leading to strokes (and other occlusive events leading to kidney failure and limb ischemia) and angina precipitating a myocardial infarct are selected. For example, regulator molecules which will diminish the adhesion ofplatelets and leukocytes, and thus diminish the occlusion events. Adhesion in this setting can be inhibited by the libraries of the invention being inserted into endothelial cells (quiescent cells, or activated by cytokines, i.e. IL-1, and growth factors, i.e. PDGF/EGF) and then screening for peptides that either: 1) downregulate adhesion molecule expression on the surface of the endothelial cells (binding assay); 2) block adhesion molecule activation on the surface of these cells (signaling assay); or 3) release in an autocrine manner peptides that block receptor binding to the cognate receptor on the adhering cell.

Embolic phenomena can also be addressed by activating proteolytic enzymes on the cell surfaces of endothelial cells, and thus releasing active enzyme which can digest blood clots. Thus, delivery of the libraries of candidate regulator molecules of the invention to endothelial cells is done, followed by standard fluorogenic assays, which will allow monitoring of proteolytic activity on the cell surface towards a known substrate. Regulator molecules can then be identified which activate specific enzymes towards specific substrates.

Arterial inflammation in the setting of vasculitis and post-infarction can be regulated by decreasing the chemotactic responses of leukocytes and mononuclear leukocytes. This can be accomplished by blocking chemotactic receptors and their responding pathways on these cells. Candidate regulator molecule libraries can be inserted into these cells, and the chemotactic response to diverse chemokines (for example, to the IL-8 family of chemokines, RANTES) inhibited in cell migration assays.

Arterial restenosis following coronary angioplasty can be controlled by regulating the proliferation of vascular intimal cells and capillary and/or arterial endothelial cells. Candidate regulator molecule libraries can be inserted into these cell types and their proliferation in response to specific stimuli monitored. One application may be intracellular regulator polypeptides which block the expression or function of c-myc and other oncogenes in smooth muscle cells to stop their proliferation. A second application may involve the expression of regulator molecule libraries in vascular smooth muscle cells to selectively induce their apoptosis. Application of small molecules derived from these peptides may require targeted drug delivery; this is available with stents, hydrogel coatings, and infusion-based catheter systems. Regulator molecules which downregulate endothelin-1A receptors or which block the release of the potent vasoconstrictor and vascular smooth muscle cell mitogen endothelin-1 may also be candidates for therapeutics. Regulator molecules can be identified in these libraries which inhibit growth of these cells, or which prevent the adhesion of other cells in the circulation known to release autocrine growth factors, such as platelets (PDGF) and mononuclear leukocytes.

The control of capillary and blood vessel growth is an important goal in order to promote increased blood flow to ischemic areas (growth), or to cut-off the blood supply (angiogenesis inhibition) of tumors. Candidate regulator molecule libraries can be inserted into capillary endothelial cells and their growth monitored. Stimuli such as low oxygen tension and varying degrees of angiogenic factors can regulate the responses, and peptides isolated that produce the appropriate phenotype. Screening for antagonism of vascular endothelial cell growth factor, important in angiogenesis, would also be useful.

The present methods are also useful in screening for decreases in atherosclerosis producing mechanisms to find peptides that regulate LDL and HDL metabolism. Candidate regulator molecule libraries can be inserted into the appropriate cells (including hepatocytes, mononuclear leukocytes, endothelial cells) and regulator molecules identified which lead to a decreased release of LDL or diminished synthesis of LDL, or conversely to an increased release of HDL or enhanced synthesis of HDL. Regulator molecules can also be identified from candidate libraries which decrease the production of oxidized LDL, which has been implicated in atherosclerosis and isolated from atherosclerotic lesions. This could occur by decreasing its expression, activating reducing systems or enzymes, or blocking the activity or production of enzymes implicated in production of oxidized LDL, such as 15-lipoxygenase in macrophage.

The present methods may also be used in screens and/or selection schemes to regulate obesity via the control of food intake mechanisms or diminishing the responses of receptor signaling pathways that regulate metabolism. Regulator molecules that induce or inhibit the responses of neuropeptide Y (NPY), cholecystokinin and galanin receptors, are particularly desirable. Candidate libraries can be inserted into cells that have these receptors cloned into them, and inhibitory regulator molecules identified that are secreted in an autocrine manner that block the signaling responses to galanin and NPY. In a similar manner, peptides can be found that regulate the leptin receptor.

The present methods are further useful in neurobiology applications. Candidate regulator molecule libraries may be used for screening for and/or selecting of anti-apoptotics for preservation of neuronal function and prevention of neuronal death. Initial screens or selections would be done in cell culture. One application would include prevention of neuronal death, by apoptosis, in cerebral ischemia resulting from stroke. Apoptosis is known to be blocked by neuronal apoptosis inhibitory protein (NAIP); screens for its upregulation, or effecting any coupled step could yield regulator molecules which selectively block neuronal apoptosis. Other applications include neurodegenerative diseases such as Alzheimer's disease and Huntington's disease.

In another embodiment, the present methods are useful in bone biology applications. Osteoclasts are known to play a key role in bone remodeling by breaking down “old” bone, so that osteoblasts can lay down “new” bone. In osteoporosis one has an imbalance of this process. Osteoclast overactivity can be regulated by inserting candidate libraries into these cells, and then looking for regulator molecules that produce: 1) a diminished processing of collagen by these cells; 2) decreased pit formation on bone chips; and 3) decreased release of calcium from bone fragments.

The present methods may also be used to screen for agonists of bone morphogenic proteins, hormone mimetics to stimulate, regulate, or enhance new bone formation (in a manner similar to parathyroid hormone and calcitonin, for example). These have use in osteoporosis, for poorly healing fractures, and to accelerate the rate of healing of new fractures. Furthermore, cell lines of connective tissue origin can be treated with candidate libraries and screened for their growth, proliferation, collagen stimulating activity, and/or proline incorporating ability on the target osteoblasts. Alternatively, candidate libraries can be expressed directly in osteoblasts or chondrocytes and screened for increased production of collagen or bone.

Additionally, the present methods are useful in skin biology applications. Keratinocyte responses to a variety of stimuli may result in psoriasis, a proliferative change in these cells. Candidate regulator molecule libraries can be inserted into cells removed from active psoriatic plaques, and regulator molecules identified which decrease the rate of growth of these cells.

The present methods are also useful in the regulation or inhibition of keloid formation (i.e. excessive scarring). Candidate regulator molecule libraries are inserted into skin connective tissue cells isolated from individuals with this condition, and regulator molecules identified that decrease proliferation, collagen formation, or proline incorporation. Results from this work can be extended to treat the excessive scarring that also occurs in burn patients. If a common peptide motif is found in the context of the keloid work, then it can be used widely in a topical manner to diminish scarring post burn.

Similarly, wound healing for diabetic ulcers and other chronic “failure to heal” conditions in the skin and extremities can be regulated by providing additional growth signals to cells which populate the skin and dermal layers. Growth factor mimetics may in fact be very useful for this condition. Candidate libraries can be inserted into skin connective tissue cells, and regulator molecules identified which promote the growth of these cells under “harsh” conditions, such as low oxygen tension, low pH, and the presence of inflammatory mediators.

Cosmeceutical applications of the present invention include the control of melanin production in skin melanocytes. A naturally occurring peptide, arbutin, is a tyrosine hydroxylase inhibitor, a key enzyme in the synthesis of melanin. Candidate libraries can be inserted into melanocytes and known stimuli that increase the synthesis of melanin applied to the cells. Regulator molecules can be identified that inhibit the synthesis of melanin under these conditions.

Additionally, the present methods are useful in endocrinology applications. The regulator molecule library technology can be applied broadly to any endocrine, growth factor, cytokine or chemokine network which involves a signaling peptide or protein that acts in either an endocrine, paracrine or autocrine manner that binds or dimerizes a receptor and activates a signaling cascade that results in a known phenotypic or functional outcome. The methods are applied so as to isolate a regulator molecule, e.g., a regulator polypeptide, which either mimics the desired hormone (i.e., insulin, leptin, calcitonin, PDGF, EGF, EPO, GMCSF, IL1-17, mimetics) or inhibits its action by either blocking the release of the hormone, blocking its binding to a specific receptor or carrier protein (for example, CRF binding protein), or inhibiting the intracellular responses of the specific target cells to that hormone. Selection of regulator molecules which increase the expression or release of hormones from the cells which normally produce them could have broad applications to conditions of hormonal deficiency.

The present methods are further useful in infectious disease applications. Viral latency (herpes viruses such as CMV, EBV, HBV, and other viruses such as HIV) and their reactivation are a significant problem, particularly in immunosuppressed patients (patients with AIDS and transplant patients). The ability to block the reactivation and spread of these viruses is an important goal. Cell lines known to harbor or be susceptible to latent viral infection can be infected with the specific virus, and then stimuli applied to these cells which have been shown to lead to reactivation and viral replication. This can be followed by measuring viral titers in the medium and scoring cells for phenotypic changes. Candidate regulator molecule libraries can then be inserted into these cells under the above conditions, and regulator molecules identified which block or diminish the growth and/or release of the virus. As with chemotherapeutics, these experiments can also be done with drugs which are only partially effective towards this outcome, and regulator molecules identified which enhance the virucidal effect of these drugs.

One example of many is the ability to block HIV-1 infection. HIV-1 requires CD4 and a co-receptor which can be one of several seven transmembrane G-protein coupled receptors. In the case of the infection of macrophages, CCR-5 is the required co-receptor, and there is strong evidence that a block on CCR-5 will result in resistance to HIV-1 infection. There are two lines of evidence for this statement. First, it is known that the natural ligands for CCR-5, the CC chemokines RANTES, MIP1a and MIP1b are responsible for CD8+ mediated resistance to HIV. Second, individuals homozygous for a mutant allele of CCR-5 are completely resistant to HIV infection. Thus, an inhibitor of the CCR-5/HIV interaction would be of enormous interest to both biologists and clinicians. The extracellular anchored constructs offer superb tools for such a discovery. Into the transmembrane, epitope tagged, glycine-serine tethered constructs (ssTM V G20 E TM), one can place a random, cyclized peptide library of the general sequence CXXXXXXXXXXC or C—(X)ₙ—C (SEQ ID NO:82). Then one infects a cell line that expresses CCR-5 with virus vectors containing this library. Using an antibody to CCR-5 one can use FACS to sort desired cells based on the binding of this antibody to the receptor. All cells which do not bind the antibody will be assumed contain inhibitors of this antibody binding site. These inhibitors, in the viral construct can be further assayed for their ability to inhibit HIV-1 entry.

Viruses are known to enter cells using specific receptors to bind to cells (for example, HIV uses CD4, coronavirus uses CD13, murine leukemia virus uses transport protein, and measles virus usesCD44) and to fuse with cells (HIV uses chemokine receptor). Candidate libraries can be inserted into target cells known to be permissive to these viruses, and regulator molecules identified which block the ability of these viruses to bind and fuse with specific target cells.

The present invention also finds use with other infectious organisms. Intracellular organisms such as Mycobacteria, Listeria, Salmonella, Pneumocystis, Yersinia, Leishmania, or Trypanosoma, can persist and replicate within cells, and become active in immunosuppressed patients. There are currently drugs on the market and in development which are either only partially effective or ineffective against these organisms. Candidate regulator molecule libraries can be inserted into specific cells infected with these organisms (pre- or post-infection), and regulator molecules identified which promote the intracellular destruction of these organisms in a manner analogous to intracellular “antibiotic peptides” similar to magainins. In addition regulator molecules can be identified which enhance the cidal properties of drugs already under investigation which have insufficient potency by themselves, but when combined with a specific regulator molecule from a candidate library, are dramatically more potent through a synergistic mechanism. Finally, regulator molecules can be identified which alter the metabolism of these intracellular organisms, in such a way as to terminate their intracellular life cycle by inhibiting a key organismal event.

Antibiotic drugs that are widely used have certain dose dependent, tissue specific toxicities. For example renal toxicity is seen with the use of gentamicin, tobramycin, and amphotericin; hepatotoxicity is seen with the use of INH and rifampin; bone marrow toxicity is seen with chloramphenicol; and platelet toxicity is seen with ticarcillin, etc. These toxicities limit their use. Candidate regulator molecule libraries can be introduced into the specific cell types where specific changes leading to cellular damage or apoptosis by the antibiotics are produced, and regulator molecules can be identified that confer protection, when these cells are treated with these specific antibiotics.

Furthermore, the present invention finds use in screening for regulator molecules that block antibiotic transport mechanisms. The rapid secretion from the blood stream of certain antibiotics limits their usefulness. For example penicillins are rapidly secreted by certain transport mechanisms in the kidney and choroid plexus in the brain. Probenecid is known to block this transport and increase serum and tissue levels. Candidate regulator molecule libraries can be inserted into specific cells derived from kidney cells and cells of the choroid plexus known to have active transport mechanisms for antibiotics. Regulator molecules can then be identified which block the active transport of specific antibiotics and thus extend the serum halflife of these drugs.

The present methods are also useful in drug toxicities and drug resistance applications. Drug toxicity is a significant clinical problem. This may manifest itself as specific tissue or cell damage with the result that the drug's effectiveness is limited. Examples include myeloablation in high dose cancer chemotherapy, damage to epithelial cells lining the airway and gut, and hair loss. Specific examples include adriamycin induced cardiomyocyte death, cisplatinin-induced kidney toxicity, vincristine-induced gut motility disorders, and cyclosporin-induced kidney damage. Candidate regulator molecule libraries can be introduced into specific cell types with characteristic drug-induced phenotypic or functional responses, in the presence of the drugs, and regulator molecules identified which reverse or protect the specific cell type against the toxic changes when exposed to the drug. These effects may manifest as blocking the drug induced apoptosis of the cell of interest, thus initial screens will be for survival of the cells in the presence of high levels of drugs or combinations of drugs used in combination chemotherapy.

Drug toxicity may be due to a specific metabolite produced in the liver or kidney which is highly toxic to specific cells, or due to drug interactions in the liver which block or enhance the metabolism of an administered drug. Candidate regulator molecule libraries can be introduced into liver or kidney cells following the exposure of these cells to the drug known to produce the toxic metabolite. Regulator polypeptides can be identified which alter how the liver or kidney cells metabolize the drug, and specific molecules identified which prevent the generation of a specific toxic metabolite. The generation of the metabolite can be followed by mass spectrometry, and phenotypic changes can be assessed by microscopy. Such a screen can also be done in cultured hepatocytes, cocultured with readout cells which are specifically sensitive to the toxic metabolite. Applications include reversible (to limit toxicity) inhibitors of enzymes involved in drug metabolism.

Multiple drug resistance, and hence tumor cell selection, outgrowth, and relapse, leads to morbidity and mortality in cancer patients. Candidate regulator molecule libraries can be introduced into tumor cell lines (primary and cultured) that have demonstrated specific or multiple drug resistance. Regulator molecules can then be identified which confer drug sensitivity when the cells are exposed to the drug of interest, or to drugs used in combination chemotherapy. The readout can be the onset of apoptosis in these cells, membrane permeability changes, the release of intracellular ions and fluorescent markers, or other methods disclosed herein. The cells in which multidrug resistance involves membrane transporters can be preloaded with fluorescent transporter substrates, and selection carried out for peptides which block the normal efflux of fluorescent drug from these cells. Candidate regulator molecule libraries are particularly suited to screening for regulator molecules which reverse poorly characterized or recently discovered intracellular mechanisms of resistance or mechanisms for which few or no chemosensitizers currently exist, such as mechanisms involving LRP (lung resistance protein). This protein has been implicated in multidrug resistance in ovarian carcinoma, metastatic malignant melanoma, and acute myeloid leukemia. Particularly interesting examples include screening for molecules which reverse more than one important resistance mechanism in a single cell, which occurs in a subset of the most drug resistant cells, which are also important targets. Applications would include screening for peptide inhibitors of both MRP (multidrug resistance related protein) and LRP for treatment of resistant cells in metastatic melanoma, for inhibitors of both p-glycoprotein and LRP in acute myeloid leukemia, and for inhibition (by any mechanism) of all three proteins for treating pan-resistant cells.

The present methods are further useful in improving the performance of existing or developmental drugs. First pass metabolism of orally administered drugs limits their oral bioavailability, and can result in diminished efficacy as well as the need to administer more drug for a desired effect. Reversible inhibitors of enzymes involved in first pass metabolism may thus be a useful adjunct enhancing the efficacy of these drugs. First pass metabolism occurs in the liver, thus inhibitors of the corresponding catabolic enzymes may enhance the effect of the cognate drugs. Reversible inhibitors would be delivered at the same time as, or slightly before, the drug of interest. Screening of candidate libraries in hepatocytes for inhibitors (by any mechanism, such as protein downregulation as well as a direct inhibition of activity) of particularly problematical isozymes would be of interest. These include the CYP3A4 isozymes of cytochrome P450, which are involved in the first pass metabolism of the anti-HIV drugs saquinavir and indinavir. Other applications could include reversible inhibitors of UDP-glucuronyltransferases, sulfotransferases, N-acetyltransferases, epoxide hydrolases, and glutathione S-transferases, depending on the drug. Screens would be done in cultured hepatocytes or liver microsomes, and could involve antibodies recognizing the specific modification performed in the liver, or cocultured readout cells, if the metabolite had a different bioactivity than the untransformed drug. The enzymes modifying the drug would not necessarily have to be known, if screening was for lack of alteration of the drug.

The present methods are also useful in immunobiology, inflammation, and allergic response applications. Selective regulation of T lymphocyte responses is a desired goal in order to modulate immune-mediated diseases in a specific manner. Candidate regulator molecule libraries can be introduced into specific T cell subsets (TH1, TH2, CD4+, CD8+, and others) and the responses which characterize those subsets (cytokine generation, cytotoxicity, proliferation in response to antigen being presented by a mononuclear leukocyte, and others) modified by members of the library. Regulator molecules can be identified which increase or diminish the known T cell subset physiologic response. This approach will be useful in any number of conditions, including: 1) autoimmune diseases where one wants to induce a tolerant state (select a peptide that inhibits T cell subset from recognizing a self-antigen bearing cell); 2) allergic diseases where one wants to decrease the stimulation of IgE producing cells (select peptide which blocks release from T cell subsets of specific B-cell stimulating cytokines which induce switch to IgE production); 3) in transplant patients where one wants to induce selective immuno suppression (select peptide that diminishes proliferative responses of host T cells to foreign antigens); 4) in lymphoproliferative states where one wants to inhibit the growth or sensitize a specific T cell tumor to chemotherapy and/or radiation; 5) in tumor surveillance where one wants to inhibit the killing of cytotoxic T cells by Fas ligand bearing tumor cells; and 5) in T cell mediated inflammatory diseases such as Rheumatoid arthritis, Connective tissue diseases (SLE), Multiple sclerosis, and inflammatory bowel disease, where one wants to inhibit the proliferation of disease-causing T cells (promote their selective apoptosis) and the resulting selective destruction of target tissues (cartilage, connective tissue, oligodendrocytes, gut endothelial cells, respectively).

Regulation of B cell responses will permit a more selective modulation of the type and amount of immunoglobulin made and secreted by specific B cell subsets. Candidate regulator molecule libraries can be inserted into B cells and regulator molecules identified which inhibit the release and synthesis of a specific immunoglobulin. This may be useful in autoimmune diseases characterized by the overproduction of auto antibodies and the production of allergy causing antibodies, such as IgE. Molecules can also be identified which inhibit or enhance the binding of a specific immunoglobulin subclass to a specific antigen either foreign of self. Finally, regulator molecules can be identified which inhibit the binding of a specific immunoglobulin subclass to its receptor on specific cell types.

Similarly, regulator molecules which affect cytokine production may be identified, generally using two cell systems. For example, cytokine production from macrophages, monocytes, etc. may be evaluated. Similarly, molecules which mimic cytokines, for example erythropoetin and IL1-17, may be identified, or molecules that bind cytokines such as TNF-α, before they bind their receptor.

Antigen processing by mononuclear leukocytes (ML) is an important early step in the immune system's ability to recognize and eliminate foreign proteins. Candidate regulator molecule libraries can be inserted into ML cell lines and molecules identified which alter the intracellular processing of foreign peptides and sequence of the foreign peptide that is presented to T cells by MLs on their cell surface in the context of Class II MHC. One can look for members of the library that enhance immune responses of a particular T cell subset (for example, the peptide would in fact work as a vaccine), or look for a library member that binds more tightly to MHC, thus displacing naturally occurring peptides, but nonetheless the agent would be less immunogenic (less stimulatory to a specific T cell clone). This agent would in fact induce immune tolerance and/or diminish immune responses to foreign proteins. This approach could be used in transplantation, autoimmune diseases, and allergic diseases.

The release of inflammatory mediators (cytokines, leukotrienes, prostaglandins, platelet activating factor, histamine, neuropeptides, and other peptide and lipid mediators) is a key element in maintaining and amplifying aberrant immune responses. Candidate regulator molecule libraries can be inserted into MLs, mast cells, eosinophils, and other cells participating in a specific inflammatory response, and regulator molecules identified which inhibit the synthesis, release and binding to the cognate receptor of each of these types of mediators.

The present methods are also useful in biotechnology applications. Candidate regulator molecule library expression in mammalian cells can also be considered for other pharmaceutical-related applications, such as modification of protein expression, protein folding, or protein secretion. One such example would be in commercial production of protein pharmaceuticals in CHO or other cells. Candidate regulator molecule libraries resulting in regulator molecules which select for an increased cell growth rate (perhaps peptides mimicking growth factors or acting as agonists of growth factor signal transduction pathways), for pathogen resistance (see previous section), for lack of sialylation or glycosylation (by blocking glycotransferases or rerouting trafficking of the protein in the cell), for allowing growth on autoclaved media, or for growth in serum free media, would all increase productivity and decrease costs in the production of protein pharmaceuticals.

Random regulator polypeptides displayed on the surface of circulating cells can be used as tools to identify organ, tissue, and cell specific peptide targeting sequences. Any cell introduced into the bloodstream of an animal expressing a library targeted to the cell surface can be selected for specific organ and tissue targeting. The regulator polypeptide sequence identified can then be coupled to an antibody, enzyme, drug, imaging agent or substance for which organ targeting is desired.

Other regulator molecules which may be identified using the present invention include: 1) regulator molecules which block the activity of transcription factors, using cell lines with reporter genes; 2) regulator molecules which block the interaction of two known proteins in cells, using the absence of normal cellular functions, the mammalian two hybrid system or fluorescence resonance energy transfer mechanisms for detection; and 3) regulator molecules may be identified by tethering a random peptide to a protein binding region to allow interactions with molecules sterically close, i.e., within a signalling pathway, to localize the effects to a functional area of interest.

A publication describing use of the fibronectin type III domain (FN3) as a specific molecular scaffold on which to display random candidate peptides for optimal effect is Koide, A. et al. J. Mol. Biol 284:1141-1151(1988). Additionally, there are several alternatives available including “minibody” (Pessi, A. et al., Nature 362:367-369 (1993)), tendamistat (McConnell, S. J. and Hoess, R. H. J. Mol. Biol. 250:460-470 (1995)), and “camelized” VH domain (Davies J. and Riechmann, L. BiolTechnology 13:475-479 (1995)). Other scaffolds that are not based on the immunoglobulin like folded structure are reviewed in Nygren, P. A. and Uhlen, M. Curr. Opin. Struct. Biol. 7:463-469 (1997). U.S. Pat. No. 6,153,380 describes additional scaffolds, fusion partners, etc., and methods relating to peptide and RNA regulator molecules.

In one embodiment, a method is provided to screen and/or select for regulator molecules using a system where cell death is induced upon expression of a selectable gene product encoding and/or comprising a cytotoxic T cell (CTL) epitope. The selectable gene product encoding the CTL epitope is placed in operable association with a target transcriptional regulatory region which is induced and/or suppressed upon expression of an appropriate regulator molecule. Upon expression of a desired regulator molecule, the CTL epitope is expressed on the surface of the host cell in the context of a defined MHC molecule which is also expressed on the surface of the host cell. The cells are contacted with epitope-specific CTLs which recognize the CTL epitope in the context of the defined MHC molecule, and the cells expressing the CTL epitope rapidly undergo a lytic event. Methods of selecting and recovering host cells expressing specific CTL epitopes are further disclosed, for example, in Zauderer, PCT Publication No. WO 00/028016.

Selection of the host cells is accomplished through recovering those cells, or the contents thereof, which have succumbed to cell death and/or have undergone a lytic event. For example, if host cells are chosen which grow attached to a solid support, those host cells which succumb to cell death and/or undergo a lytic event will be released from the support and can be recovered in the cell supernatant. Alternatively virus particles released from host cells which have succumbed to cell death and/or undergone a lytic event may be recovered from the cell supernatant.

According to this embodiment, the MHC molecule expressed on the surface of the host cells may be either a class I MHC molecule or a class II MHC molecule. To isolate a regulator molecule by virtue of expressing an epitope recognized by a human CD8⁺ CTL, it is preferable to use a host cell which expresses human class I MHC molecules, and to isolate a regulator molecule by virtue of expressing an epitope recognized by a human CD4⁺ CTL, it is preferable to use a host cell which expresses human class II MHC molecules, to allow the CTL to recognize the epitope in association with the appropriate MHC molecules. In a particularly preferred embodiment, the MHC molecule expressed on the host cells is an H-2K^{d} molecule, and the CTL epitope which is expressed is the peptide GYKAGMIHI, designated herein as SEQ ID NO:115.

In another embodiment, a method is provided a method is provided to induce cell death upon expression of a selectable “suicide” gene product encoding a “suicide” protein. The selectable gene product encoding the suicide protein is placed in operable association with a target transcriptional regulatory region which is induced and/or suppressed upon expression of an appropriate regulator molecule. By “suicide gene” is meant a nucleic acid molecule which causes cell death when expressed, but expressing, e.g., a “suicide protein.” Polynucleotides useful as suicide genes include many cell death-inducing sequences which are known in the art. Examples of suicide genes are those which encode toxins such as Pseudomonas exotoxin A chain, diphtheria A chain, ricin A chain, abrin A chain, modeccin A chain, and alpha-sarcin. A preferred suicide gene encodes the diphtheria A toxin subunit. Upon expression of a desired regulator molecule, the promoter of the suicide gene is induced, thereby allowing expression of the suicide gene, and thereby promoting cell death.

In another embodiment, a screening method is provided to recover polynucleotides encoding regulator molecules of interest based upon expression of a selectable gene product encoding and/or comprising a reporter protein. The selectable gene product encoding an easily detectable reporter protein, e.g., beta-galactosidase, green fluorescent protein, or luciferase, is placed in operable association with a target transcriptional regulatory region which is induced and/or suppressed upon expression of an appropriate regulator molecule. Expression of the reporter protein is directly or indirectly upregulated or downregulated as a result of expression of a desired regulator molecule. Pools of host cells expressing candidate regulator molecules are screened or selected for expression of the reporter molecule, or lack thereof, detected in that pool.

Any suitable reporter molecule may be used in these methods, the choice depending upon the host cells used, the detection instruments available, and the ease of detection desired. Suitable reporter molecules include, but are not limited to luciferase, green fluorescent protein, and beta-galactosidase.

In those embodiments involving the use of virus vectors to deliver candidate regulator molecules, kinetic considerations dictate that expression of the reporter construct, suicide gene, or CTL epitope take place prior to the induction of cytopathic effect (CPE) by the virus. Nonetheless, it is preferred that expression of a detectable reporter molecule, suicide gene, or CTL epitope occurs within a period between about 1 hour to about 4 days after, for example, introduction of the library, so as to precede induction of CPE. More preferably, reporter molecule, suicide gene, or CTL epitope expression occurs within about 1 hour about 2 hours, about 3 hours about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 24 hours, about 28 hours, about 32 hours, about 36 hours, about 40 hours, about 44 hours, or about 48 hours after contacting the host cells with antigen. Even more preferably reporter molecule expression occurs within about 12 hours of, for example, introducing the library, etc.

As used herein, a “solid support” or a “solid substrate” is any support capable of binding a cell or antigen, which may be in any of various forms, as is known in the art. Well-known supports include tissue culture plastic, glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration as long as the coupled molecule is capable of binding to a cell. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. The support configuration may include a tube, bead, microbead, well, plate, tissue culture plate, petri plate, microplate, microtiter plate, flask, stick, strip, vial, paddle, etc., etc. A solid support may be magnetic or non-magnetic. Those skilled in the art will know many other suitable carriers for binding cells or antigens, or will be able to readily ascertain the same.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

### Trimolecular Recombination

1.1 Production of an Expression Library. This example describes a tri-molecular recombination method employing modified vaccinia virus vectors and related transfer plasmids that generates close to 100% recombinant vaccinia virus and, for the first time, allows efficient construction of a representative DNA library in vaccinia virus. The trimolecular recombination method is illustrated in FIG. 6.

1.2 Construction of the Vectors. The previously described vaccinia virus transfer plasmid pJ/K, a pUC 13 derived plasmid with a vaccinia virus thymidine kinase gene containing an in-frame Not I site (Merchlinsky, M. et al., Virology 190:522-526), was further modified to incorporate a strong vaccinia virus promoter followed by Not I and Apa I restriction sites. Two different vectors, p7.5/tk and pEL/tk, included, respectively, either the 7.5K vaccinia virus promoter or a strong synthetic early/late (E/L) promoter (FIG. 7). The Apa I site was preceded by a strong translational initiation sequence including the ATG codon. This modification was introduced within the vaccinia virus thymidine kinase (tk) gene so that it was flanked by regulatory and coding sequences of the viral tk gene. The modifications within the tk gene of these two new plasmid vectors were transferred by homologous recombination in the flanking tk sequences into the genome of the Vaccinia Virus WR strain derived vNotI⁻vector to generate new viral vectors v7.5/tk and vEL/tk. Importantly, following Not I and Apa I restriction endonuclease digestion of these viral vectors, two large viral DNA fragments were isolated each including a separate non-homologous segment of the vaccinia tk gene and together comprising all the genes required for assembly of infectious viral particles. Further details regarding the construction and characterization of these vectors and their alternative use for direct ligation of DNA fragments in vaccinia virus are described in Zauderer, PCT Publication WO 00/028016.

1.3 Generation of an Increased Frequency of Vaccinia Virus Recombinants. Standard methods for generation of recombinants in vaccinia virus exploit homologous recombination between a recombinant vaccinia transfer plasmid and the viral genome. Table 3 shows the results of a model experiment in which the frequency of homologous recombination following transfection of a recombinant transfer plasmid into vaccinia virus infected cells was assayed under standard conditions. To facilitate functional assays, a minigene encoding the immunodominant 257-264 peptide epitope of ovalbumin in association with H-2K^{b} was inserted at the Not 1 site in the transfer plasmid tk gene. As a result of homologous recombination, the disrupted tk gene is substituted for the wild type viral tk+ gene in any recombinant virus. This serves as a marker for recombination since tk− human 143B cells infected with tk− virus are, in contrast to cells infected with wild type tk+ virus, resistant to the toxic effect of BrdU. Recombinant virus can be scored by the viral pfu on 143B cells cultured in the presence of 125 mM BrdU.

The frequency of recombinants derived in this fashion is of the order of 0.1% (Table 3).

TABLE 3
Generation of Recombinant Vaccinia Virus
by Standard Homologous Recombination

| | | Titer w/o | Titer w/ | % |
|---|---|---|---|---|
| Virus* | DNA | BrdU | BrdU | Recombinant** |
| vaccinia | — | 4.6 × 10⁷ | 3.0 × 10³ | 0.006 |
| vaccinia | 30 ng pE/Lova | 3.7 × 10⁷ | 3.2 × 10⁴ | 0.086 |
| vaccinia | 300 ng pE/Lova | 2.7 × 10⁷ | 1.5 × 10⁴ | 0.056 |

*vaccinia virus strain vNotI
**% Recombinant = (Titer with BrdU/Titer without BrdU) × 100

This recombination frequency is too low to permit efficient construction of a cDNA library in a vaccinia vector. The following two procedures were used to generate an increased frequency of vaccinia virus recombinants.

(1) One factor limiting the frequency of viral recombinants generated by homologous recombination following transfection of a plasmid transfer vector into vaccinia virus infected cells is that viral infection is highly efficient whereas plasmid DNA transfection is relatively inefficient. As a result many infected cells do not take up recombinant plasmids and are, therefore, capable of producing only wild type virus. In order to reduce this dilution of recombinant efficiency, a mixture of naked viral DNA and recombinant plasmid DNA was transfected into Fowl Pox Virus (FPV) infected mammalian cells. As previously described by others (Scheiflinger, F. et al., Proc. Natl. Acad. Sci. USA 89:9977-1981 (1992)), FPV does not replicate in mammalian cells but provides necessary helper functions required for packaging mature vaccinia virus particles in cells transfected with non-infectious naked vaccinia DNA. This modification of the homologous recombination technique alone increased the frequency of viral recombinants approximately 35 fold to 3.5% (Table 4).

TABLE 4
Generation of Recombinant Vaccinia Virus
by Modified Homologous Recombination

| | | Titer w/o | Titer w/ | % |
|---|---|---|---|---|
| Virus | DNA | BrdU | BrdU | Recombinant* |
| PFV | None | 0 | 0 | 0 |
| None | vaccinia WR | 0 | 0 | 0 |
| PFV | vaccinia WR | 8.9 × 10⁶ | 2.0 × 10² | 0.002 |
| PFV | vaccinia WR + | 5.3 × 10⁶ | 1.2 × 10⁵ | 2.264 |
| | pE/Lova (1:1) | | | |
| PFV | vaccinia WR + | 8.4 × 10⁵ | 3.0 × 10⁴ | 3.571 |
| | pE/Lova (1:10) | | | |

*% Recombinant = (Titer with BrdU/Titer without BrdU) × 100

Table 4. Confluent monolayers of BSC1 cells (5×10⁵ cells/well) were infected with moi=1.0 of fowlpox virus strain HP 1. Two hours later supernatant was removed, cells were washed 2× with Opti-Mem I media, and transfected using lipofectamine with 600 ng vaccinia strain WR genomic DNA either alone, or with 1:1 or 1:10 (vaccinia:plasmid) molar ratios of plasmid pE/Lova. This plasmid contains a fragment of the ovalbumin cDNA, which encodes the SIINFEKL epitope (SEQ ID NO:116), known to bind with high affinity to the mouse class I MHC molecule K^{b}. Expression of this minigene is controlled by a strong, synthetic Early/Late vaccinia promoter. This insert is flanked by vaccinia tk DNA. Three days later cells were harvested, and virus extracted by three cycles of freeze/thaw in dry ice isopropanol/37° C. water bath. Crude virus stocks were titered by plaque assay on human TK- 143B cells with and without BrdU.

A further significant increase in the frequency of viral recombinants was obtained by transfection of FPV infected cells with a mixture of recombinant plasmids and the two large approximately 80 kilobases and 100 kilobases fragments of vaccinia virus v7.5/tk DNA produced by digestion with Not I and Apa I restriction endonucleases. Because the Not I and Apa I sites have been introduced into the tk gene, each of these large vaccinia DNA arms includes a fragment of the tk gene. Since there is no homology between the two tk gene fragments, the only way the two vaccinia arms can be linked is by bridging through the homologous tk sequences that flank the inserts in the recombinant transfer plasmid. The results in Table 5 show that >99% of infectious vaccinia virus produced in triply transfected cells is recombinant for a DNA insert as determined by BrdU resistance of infected tk- cells.

TABLE 5
Generation of 100% Recombinant Vaccinia Virus
Using Tri-Molecular Recombination

| | | Titer w/o | Titer w/ | % |
|---|---|---|---|---|
| Virus | DNA | BrdU | BrdU | Recombinant* |
| PFV | Uncut v7.5/tk | 2.5 × 10⁶ | 6.0 × 10³ | 0.24 |
| PFV | NotI/Apal v7.5/tk arms | 2.0 × 10² | 0 | 0 |
| PFV | NotI/Apal v7.5/tk arms + | 6.8 × 10⁴ | 7.4 × 10⁴ | 100 |
| | pE/Lova (1:1) | | | |

*% Recombinant = (Titer with BrdU/Titer without BrdU) × 100

Table 5. Genomic DNA from vaccinia strain V7.5/tk (1.2 micrograms) was digested with ApaI and NotI restriction endonucleases. The digested DNA was divided in half. One of the pools was mixed with a 1:1 (vaccinia:plasmid) molar ratio of pE/Lova. This plasmid contains a fragment of the ovalbumin cDNA, which encodes the SIINFEKL epitope, (SEQ ID NO:116) known to bind with high affinity to the mouse class I MHC molecule K^{b}. Expression of this minigene is controlled by a strong, synthetic Early/Late vaccinia promoter. This insert is flanked by vaccinia tk DNA. DNA was transfected using lipofectamine into confluent monolayers (5×105 cells/well) of BSC1 cells, which had been infected 2 hours previously with moi=1.0 FPV. One sample was transfected with 600 ng untreated genomic V7.5/tk DNA. Three days later cells were harvested, and the virus was extracted by three cycles of freeze/thaw in dry ice isopropanol/37° C. water bath. Crude viral stocks were plaqued on TK- 143 B cells with and without BrdU selection.

1.4 Construction of a Representative cDNA Library in Vaccinia Virus. A cDNA library is constructed in the vaccinia vector to demonstrate representative expression of known cellular mRNA sequences. Additional modifications have been introduced into the p7.5/tk transfer plasmid and v7.5/tk viral vector to enhance the efficiency of recombinant expression in infected cells. These include introduction of translation initiation sites in three different reading frames and of both translational and transcriptional stop signals as well as additional restriction sites for DNA insertion.

First, the HindIll J fragment (vaccinia tk gene) of p7.5/tk was subcloned from this plasmid into the HindIII site of pBS phagemid (Stratagene) creating pBS.Vtk.

Second, a portion of the original multiple cloning site of pBS.Vtk was removed by digesting the plasmid with SmaI and PstI, treating with Mung Bean Nuclease, and ligating back to itself, generating pBS.Vtk.MCS-. This treatment removed the unique SmaI, BamHI, SalI, and PstI sites from pBS.Vtk.

Third, the object at this point was to introduce a new multiple cloning site downstream of the 7.5k promoter in pBS.Vtk.MCS-. The new multiple cloning site was generated by PCR using 4 different upstream primers, and a common downstream primer. Together, these 4 PCR products would contain either no ATG start codon, or an ATG start codon in each of the three possible reading frames. In addition, each PCR product contains at its 3 prime end, translation stop codons in all three reading frames, and a vaccinia virus transcription double stop signal. These 4 PCR products were ligated separately into the NotI/ ApaI sites of pBS.Vtk.MCS-, generating the 4 vectors, p7.5/ATGO/tk, p7.5/ATG1/tk, p7.5/ATG3/tk, and p7.5/ATG4/tk whose sequence modifications relative to the p7.5/tk vector are shown in FIG. 8. Each vector includes unique BamHI, SmaI, PstI, and SalI sites for cloning DNA inserts that employ either their own endogenous translation initiation site (in vector p7.5/ATG0/tk) or make use of a vector translation initiation site in any one of the three possible reading frames (p7.5/ATG1/tk, p7.5/ATG3/tk, and p7.5/ATG4/tk).

In a model experiment cDNA was synthesized from poly-A+ mRNA of a murine tumor cell line (BCA39) and ligated into each of the four modified p7.5/tk transfer plasmids. The transfer plasmid is amplified by passage through procaryotic host cells such as E. coli as described herein or as otherwise known in the art. Twenty micrograms of Not I and Apa I digested v/tk vaccinia virus DNA arms and an equimolar mixture of the four recombinant plasmid cDNA libraries was transfected into FPV helper virus infected BSC-1 cells for tri-molecular recombination. The virus harvested had a total titer of 6×10⁶ pfu of which greater than 90% were BrdU resistant.

In order to characterize the size distribution of cDNA inserts in the recombinant vaccinia library, individual isolated plaques were picked using a sterile pasteur pipette and transferred to 1.5 ml tubes containing 100 μl Phosphate Buffered Saline (PBS). Virus was released from the cells by three cycles of freeze/thaw in dry ice/isopropanol and in a 37° C. water bath. Approximately one third of each virus plaque was used to infect one well of a 12 well plate containing tk− human 143B cells in 250 μl final volume. At the end of the two hour infection period each well was overlayed with 1 ml DMEM with 2.5% fetal bovine serum (DMEM-2.5) and with BUdR sufficient to bring the final concentration to 125 μg/ml. Cells were incubated in a CO₂ incubator at 37° C. for three days. On the third day the cells were harvested, pelleted by centrifugation, and resuspended in 500 μl PBS. Virus was released from the cells by three cycles of freeze/thaw as described above. Twenty percent of each virus stock was used to infect a confluent monolayer of BSC-1 cells in a 50 mm tissue culture dish in a final volume of 3 ml DMEM-2.5. At the end of the two hour infection period the cells were overlayed with 3 ml of DMEM-2.5. Cells were incubated in a CO₂ incubator at 37° C. for three days. On the third day the cells were harvested, pelleted by centrifugation, and resuspended in 300 μl PBS. Virus was released from the cells by three cycles of freeze/thaw as described above. One hundred microliters of crude virus stock was transferred to a 1.5 ml tube, an equal volume of melted 2% low melting point agarose was added, and the virus/agarose mixture was transferred into a pulsed field gel sample block. When the agar worms were solidified they were removed from the sample block and cut into three equal sections. All three sections were transferred to the same 1.5 ml tube, and 2501 μl of 0.5M EDTA, 1% Sarkosyl, 0.5 mg/ml Proteinase K was added. The worms were incubated in this solution at 37° C. for 24 hours. The worms were washed several times in 500 μl 0.5× TBE buffer, and one section of each worm was transferred to a well of a 1% low melting point agarose gel. After the worms were added the wells were sealed by adding additional melted 1% low melting point agarose. This gel was then electorphoresed in a Bio-Rad pulsed field gel electrophoresis apparatus at 200 volts, 8 second pulse times, in 0.5× TBE for 16 hours. The gel was stained in ethidium bromide, and portions of agarose containing vaccinia genomic DNA were excised from the gel and transferred to a 1.5 ml tube. Vaccinia DNA was purified from the agarose using β-Agarase (Gibco) following the recommendations of the manufacturer. Purified vaccinia DNA was resuspended in 50 μl ddH₂O. One microliter of each DNA stock was used as the template for a Polymerase Chain Reaction (PCR) using vaccinia TK specific primers MM428 and MM430 (which flank the site of insertion) and Klentaq Polymerase (Clontech) following the recommendations of the manufacturer in a 20 μl final volume. Reaction conditions included an initial denaturation step at 95° C. for 5 minutes, followed by 30 cycles of: 94° C. 30 seconds, 55° C. 30 seconds, 68° C. 3 minutes. Two and a half microliters of each PCR reaction was resolved on a 1% agarose gel, and stained with ethidium bromide. Amplified fragments of diverse sizes were observed. When corrected for flanking vector sequences amplified in PCR the inserts range in size between 300 and 2500 bp.

Representative expression of gene products in this library was established by demonstrating that the frequency of specific cDNA recombinants in the vaccinia library was indistinguishable from the frequency with which recombinants of the same cDNA occur in a standard plasmid library. This is illustrated in Table 6 for an IAP sequence that was previously shown to be upregulated in murine tumors.

Twenty separate pools with an average of either 800 or 200 viral pfu from the vaccinia library were amplified by infecting microcultures of 143B tk- cells in the presence of BDUR. DNA was extracted from each infected culture after three days and assayed by PCR with sequence specific primers for the presence of a previously characterized endogenous retrovirus (IAP, intracisternal A particle) sequence. Poisson analysis of the frequency of positive pools indicates a frequency of one IAP recombinant for approximately every 500 viral pfu (Table 6). Similarly, twenty separate pools with an average of either 1,400 or 275 bacterial cfu from the plasmid library were amplified by transformation of DH5 a bacteria. Plasmid DNA from each pool was assayed for the presence of the same IAP sequence. Poisson analysis of the frequency of positive pools indicates a frequency of one IAP recombinant for every 450 plasmids (Table 6).

TABLE 6
Limiting dilution analysis of IAP sequences in a recombinant
Vaccinia library and a conventional plasmid cDNA library

| | | | |
|---|---|---|---|
| # Wells Positive | | | |
| by PCR | F₀ | μ | Frequency |

| | |
|---|---|
| # PFU/well | Vaccinia Library |

| | | | | |
|---|---|---|---|---|
| 800 | 18/20 | 0.05 | 2.3 | 1/350 |
| 200 | 6/20 | 0.7 | 0.36 | 1/560 |

| | |
|---|---|
| # CFU/well | Plasmid Library |

| | | | | |
|---|---|---|---|---|
| 1400 | 20/20 | 0 | — | — |
| 275 | 9/20 | 0.55 | 0.6 | 1/450 |

F₀ = fraction negative wells;
μ = DNA precursors/well = −1nF₀

Similar analysis was carried out with similar results for representation of an alpha tubulin sequence in the vaccinia library. The comparable frequency of arbitrarily chosen sequences in the two libraries constructed from the same tumor cDNA suggests that although construction of the Vaccinia library is somewhat more complex and is certainly less conventional than construction of a plasmid library, it is equally representative of tumor cDNA sequences.

### Discussion

The above-described tri-molecular recombination strategy yields close to 100% viral recombinants. This is a highly significant improvement over current methods for generating viral recombinants by transfection of a plasmid transfer vector into vaccinia virus infected cells. This latter procedure yields viral recombinants at a frequency of the order of only 0.1%. The high yield of viral recombinants in tri-molecular recombination makes it possible, for the first time, to efficiently construct genomic or cDNA libraries in a vaccinia virus derived vector. In the first series of experiments a titer of 6×10⁶ recombinant virus was obtained following transfection with a mix of 20 micrograms of Not I and Apa I digested vaccinia vector arms together with an equimolar concentration of tumor cell cDNA. This technological advance creates the possibility of new and efficient screening and selection strategies for isolation of specific genomic and cDNA clones.

The tri-molecular recombination method as herein disclosed may be used with other viruses such as mammalian viruses including vaccinia and herpes viruses. Typically, two viral arms which have no homology are produced. The only way that the viral arms can be linked is by bridging through homologous sequences that flank the insert in a transfer vector such as a plasmid. When the two viral arms and the transfer vector are present in the same cell the only infectious virus produced is recombinant for a DNA insert in the transfer vector.

Libraries constructed in vaccinia and other mammalian viruses by the tri-molecular recombination method of the present invention may have similar advantages to those described here for vaccinia virus and its use in identifying target antigens in the CTL screening system of the invention. Similar advantages are expected for DNA libraries constructed in vaccinia or other mammalian viruses when carrying out more complex assays in eukaryotic cells. Such assays include but are not limited to screening for DNA encoding receptors and ligands of eukaryotic cells.

### Example 2

### Preparation of Transfer Plasmids

The transfer vectors may be prepared for cloning by known means. A preferred method involves cutting 1-5 micrograms of vector with the appropriate restriction endonucleases (for example SmaI and SalI or BamHI and SalI) in the appropriate buffers, at the appropriate temperatures for at least 2 hours. Linear digested vector is isolated by electrophoresis of the digested vector through a 0.8% agarose gel. The linear plasmid is excised from the gel and purified from agarose using methods that are well known.

Ligation. The cDNA and digested transfer vector are ligated together using well known methods. In a preferred method 50-100 ng of transfer vector is ligated with varying concentrations of cDNA using T4 DNA Ligase, using the appropriate buffer, at 14° C. for 18 to 24 hours.

Transformation. Aliquots of the ligation reactions are transformed by electroporation into E. coli bacteria such as DH10B or DH5 alpha using methods that are well known. The transformation reactions are plated onto LB agar plates containing a selective antibiotic (ampicillin) and grown for 14-18 hours at 37° C. All of the transformed bacteria are pooled together, and plasmid DNA is isolated using well known methods.

Preparation of buffers mentioned in the above description of preferred methods according to the present invention will be evident to those of skill.

### Example 3

### Introduction of Vaccinia Virus DNA Fragments and Transfer Plasmids into Tissue Culture Cells for Trimolecular Recombination

A cDNA or other library is constructed in the 4 transfer plasmids as described in Example 5, or by other art-known techniques. Trimolecular recombination is employed to transfer this cDNA library into vaccinia virus. Confluent monolayers of BSC1 cells are infected with fowlpox virus HP1 at a moi of 1-1.5. Infection is done in serum free media supplemented with 0.1% Bovine Serum Albumin. The BSC1 cells may be in 12 well or 6 well plates, 60 mm or 100 mm tissue culture plates, or 25 cm², 75 cm², or 150 cm² flasks. Purified DNA from v7.5/tk or vEL/tk is digested with restriction endonucleases ApaI and NotI. Following these digestions the enzymes are heat inactivated, and the digested vaccinia arms are purified using a centricon 100 column. Transfection complexes are then formed between the digested vaccinia DNA and the transfer plasmid cDNA library. A preferred method uses Lipofectamine or Lipofectamine Plus (Life Technologies, Inc.) to form these transfection complexes. Transfections in 12 well plates usually require 0.5 micrograms of digested vaccinia DNA and 10 ng to 200 ng of plasmid DNA from the library. Transfection into cells in larger culture vessels requires a proportional increase in the amounts of vaccinia DNA and transfer plasmid. Following a two hour infection at 37° C. the fowlpox is removed, and the vaccinia DNA, transfer plasmid transfection complexes are added. The cells are incubated with the transfection complexes for 3 to 5 hours, after which the transfection complexes are removed and replaced with 1 ml DMEM supplemented with 2.5% Fetal Bovine Serum. Cells are incubated in a CO² incubated at 37° C. for 3 days. After 3 days the cells are harvested, and virus is released by three cycles of freeze/thaw in dry ice/isopropanol/37° C. water bath.

### Example 4

### Transfection of Mammalian Cells

This example describes alternative methods to transfect cells with vaccinia DNA and transfer plasmid. Trimolecular recombination can be performed by transfection of digested vaccinia DNA and transfer plasmid into host cells using for example, calcium-phosphate precipitation (Graham F. L. and Van der Eb, A. J. Virology 52: 456-467 (1973), Chen, C. and Okayama, H. Mol. Cell. Biol. 7:2745-2752 (1987)), DEAE-Dextran (Sussman, D. J. and Milman, G. Mol. Cell. Biol. 4:1641-1643 (1984)), or electroporation (Wong, T. K. and Neumann, E. Biochem. Biophys. Res. Commun. 107: 584-587 (1982), Neumann, E. et al., EMBO J. 1: 841-845 (1982)).

### Example 5

### Trimolecular Recombination Methods

### 5.1 Poxvirus Vectors

As noted above, a preferred virus vector for use in the present invention is a poxvirus vector. “Poxvirus” includes any member of the family Poxviridae, including the subfamililes Chordopoxviridae (vertebrate poxviruses) and Entomopoxviridae (insect poxviruses). See, for example, B. Moss in: Virology, 2d Edition, Fields, B. N. and Knipe, D. M. et al., Eds., Raven Press, p. 2080 (1990). The chordopoxviruses comprise, inter alia, the following genera: Orthopoxvirus (e.g., vaccinia, variolavirus, raccoon poxvirus); Avipoxvirus (e.g., fowlpox); Capripoxvirus (e.g, sheeppox) Leporipoxvirus (e.g., rabbit (Shope) fibroma, and myxoma); and Suipoxvirus (e.g., swinepox). The entomopoxviruses comprise three genera: A, B and C. In the present invention, orthopoxviruses are preferred. Vaccinia virus is the prototype orthopoxvirus, and has been developed and is well-characterized as a vector for the expression of heterologous proteins. In the present invention, vaccinia virus vectors, particularly those that have been developed to perform trimolecular recombination, are preferred. However, other orthopoxviruses, in particular, raccoon poxvirus have also been developed as vectors and in some applications, have superior qualities.

Poxviruses are distinguished by their large size and complexity, and contain similarly large and complex genomes. Notably, poxviruses replication takes place entirely within the cytoplasm of a host cell. The central portions of poxvirus genomes are similar, while the terminal portions of the virus genomes are characterized by more variability. Accordingly, it is thought that the central portion of poxvirus genomes carry genes responsible for essential functions common to all poxviruses, such as replication. By contrast, the terminal portions of poxvirus genomes appear responsible for characteristics such as pathogenicity and host range, which vary among the different poxviruses, and may be more likely to be non-essential for virus replication in tissue culture. It follows that if a poxvirus genome is to be modified by the rearrangement or removal of DNA fragments or the introduction of exogenous DNA fragments, the portion of the naturally-occurring DNA which is rearranged, removed, or disrupted by the introduction of exogenous DNA is preferably in the more distal regions though to be non-essential for replication of the virus and production if infectious virions in tissue culture.

The naturally-occurring vaccinia virus genome is a cross-linked, double stranded linear DNA molecule, of about 186,000 base pairs (bp), which is characterized by inverted terminal repeats. The genome of vaccinia virus has been completely sequenced, but the functions of most gene products remain unknown. Goebel, S. J. et al., Virology 179:247-266, 517-563 (1990); Johnson, G. P. et al., Virology 196:381-401 (1993). A variety of non-essential regions have been identified in the vaccinia virus genome. See, e.g., Perkus, M. E. et al.,Virology 152:285-97 (1986); and Kotwal, G. J. and Moss B. Virology 167:524-37 (1988).

In those embodiments where poxvirus vectors, in particular vaccinia virus vectors, are used to express regulator molecules, any suitable poxvirus vector may be used. It is preferred that the libraries of regulator molecules be carried in a region of the vector which is non-essential for growth and replication of the vector so that infectious viruses are produced. Although a variety of non-essential regions of the vaccinia virus genome have been characterized, the most widely used locus for insertion of foreign genes is the thymidine kinase locus, located in the HindIII J fragment in the genome. In certain preferred vaccinia virus vectors, the tk locus has been engineered to contain one or two unique restriction enzyme sites, allowing for convenient use of the trimolecular recombination method of library generation. See infra, and also Zauderer, WO 00/028016, published May 18, 2000, and Zauderer, WO 01/72995, published Oct. 4, 2001.

Libraries of polynucleotides encoding regulator molecules are inserted into poxvirus vectors, particularly vaccinia virus vectors, under operable association with a transcriptional control region which functions in the cytoplasm of a poxvirus-infected cell.

Poxvirus transcriptional control regions comprise a promoter and a transcription termination signal. Gene expression in poxviruses is temporally regulated, and promoters for early, intermediate, and late genes possess varying structures. Certain poxvirus genes are expressed constitutively, and promoters for these “early-late” genes bear hybrid structures. Synthetic early-late promoters have also been developed. See Hammond J. M. et al., J. Virol. Methods 66:135-138(1997; Chakrabarti S. et al, Biotechniques 23:1094-7 (1997). In the present invention, any poxvirus promoter may be used, but use of early, late, or constitutive promoters may be desirable based on the host cell and/or selection scheme chosen. Typically, the use of constitutive promoters is preferred.

Examples of early promoters include the 7.5-kD promoter (also a late promoter), the DNA pol promoter, the tk promoter, the RNA pol promoter, the 19-kD promoter, the 22-kD promoter, the 42-kD promoter, the 37-kD promoter, the 87-kD promoter, the H3′ promoter, the H6 promoter, the D1 promoter, the D4 promoter, the D5 promoter, the D9 promoter, the D12 promoter, the 13 promoter, the M1 promoter, and the N2 promoter. See, e.g., Moss, B., “Poxviridae and their Replication” in Virology, 2d Edition, Fields, B. N. and Knipe, D. M. et al., Eds., Raven Press, p.2088 (1990). Early genes transcribed in vaccinia virus and other poxviruses recognize the transcription termination signal TTTTTNT, where N can be any nucleotide. Transcription normally terminates approximately 50 bp upstream of this signal. Accordingly, if heterologous genes are to be expressed from poxvirus early promoters, care must be taken to eliminate occurrences of this signal in the coding regions for those genes. See, e.g., Earl, P. L. et al., J. Virol. 64:2448-51 (1990).

Examples of late promoters include the 7.5-kD promoter, the MIL promoter, the 37-kD promoter, the 11-kD promotor, the 11L promoter, the 12L promoter, the 13L promoter, the 15L promoter, the 17L promoter, the 28-kD promoter, the H1L promoter, the H3L promoter, the H5L promoter, the H6L promoter, the H8L promoter, the D11L promoter, the D12L promotor, the D13L promoter, the A1L promoter, the A2L promoter, the A3L promoter, and the P4b promoter. See, e.g., Moss, B., “Poxviridae and their Replication” in Virology, 2d Edition, Fields, B. N. and Knipe, D. M. et al., Eds., Raven Press, p.2090 (1990). The late promoters apparently do not recognize the transcription termination signal recognized by early promoters.

Preferred constitutive promoters for use in the present invention include the synthetic early-late promoters described by Hammond and Chakrabarti, the MH-5 early-late promoter, and the 7.5-kD or “p7.5” promoter. Examples utilizing these promoters are disclosed herein.

### 5.2 Attenuated, Defective, and Inactivated Virus

As will be discussed in more detail below, certain selection and screening methods based on host cell death require that the mechanisms leading to cell death occur prior to any cytopathic effect (CPE) caused by virus infection. The kinetics of the onset of CPE in virus-infected cells is dependent on the virus used, the multiplicity of infection, and the type of host cell. For example, in many tissue culture lines infected with vaccinia virus at an MOI of about 1, CPE is not significant until well after 48 to 72 hours post-infection. This allows a 2 to 3 day time frame for high level expression of regulator molecules, and screening or selection independent of CPE caused by the vector. However, this time frame may not be sufficient for certain selection methods, especially where higher MOIs are used, and further, the time before the onset of CPE may be shorter in a desired cell line. There is, therefore, a need for virus vectors, particularly poxvirus vectors such as vaccinia virus, with attenuated cytopathic effects so that, wherever necessary, the time frame of selection can be extended.

For example, certain attenuations are achieved through genetic mutation. Many vaccinia virus mutants have been characterized. These may be fully defective mutants, i.e., the production of infectious virus particles requires helper virus, or they may be conditional mutants, e.g., temperature sensitive mutants. Conditional mutants are particularly preferred, in that the virus-infected host cells can be maintained in a non-permissive environment, e.g., at a non-permissive temperature, during the period where host gene expression is required, and then shifted to a permissive environment, e.g., a permissive temperature, to allow virus particles to be produced. Alternatively, a fully infectious virus may be “attenuated” by chemical inhibitors which reversibly block virus replication at defined points in the infection cycle. Chemical inhibitors include, but are not limited to hydroxyurea and 5-fluorodeoxyuridine. Virus-infected host cells are maintained in the chemical inhibitor during the period where host gene expression is required, and then the chemical inhibitor is removed to allow virus particles to be produced. A number of attenuated poxviruses, in particular vaccinia viruses, have been developed. For example, modified vaccinia Ankara (MVA) is a highly attenuated strain of vaccinia virus that was derived during over 570 passages in primary chick embryo fibroblasts (Mayr, A. et al., Infection 3:6-14 (1975)). The recovered virus deleted approximately 15% of the wild type vaccinia DNA which profoundly affects the host range restriction of the virus. MVA cannot replicate or replicates very inefficiently in most mammalian cell lines. A unique feature of the host range restriction is that the block in non-permissive cells occurs at a relatively late stage of the replication cycle. Expression of viral late genes is relatively unimpaired but virion morphogenesis is interrupted (Suter, G. and Moss, B., Proc Natl Acad Sci USA 89:10847-51 (1992); Carroll, M. W. and Moss, B., Virology 238:198-211 (1997)). The high levels of viral protein synthesis even in non-permissive host cells make MVA an especially safe and efficient expression vector. However, because MVA cannot complete the infectious cycle in most mammalian cells, in order to recover infectious virus for multiple cycles of selection it will be necessary to complement the MVA deficiency by coinfection or superinfection with a helper virus that is itself deficient and that can be subsequently separated from infectious MVA recombinants by differential expansion at low MOI in MVA permissive host cells.

As an alternative to MVA, some strains of vaccinia virus that are deficient in an essential early gene have been shown to have greatly reduced inhibitory effects on host cell protein synthesis. Attenuated poxviruses which lack defined essential early genes have also been described. See, e.g., U.S. Pat. No. 5,766,882, by Falkner, et al. Examples of essential early genes which may be rendered defective include, but are not limited to the vaccinia virus 17L, F18R, D13L, D6R, A8L, J1R, E7L, F11L, E4L, I1L, J3R, J4R, H7R, and A6R genes. A preferred essential early gene to render defective is the D4R gene, which encodes a uracil DNA glycosylase enzyme. Vaccinia viruses defective in defined essential genes are easily propagated in complementing cell lines which provides the essential gene product.

As used herein, the term “complementation” refers to a restoration of a lost function in trans by another source, such as a host cell, transgenic animal or helper virus. The loss of function is caused by loss by the defective virus of the gene product responsible for the function. Thus, a defective poxvirus is a non-viable form of a parental poxvirus, and is a form that can become viable in the presence of complementation. The host cell, transgenic animal or helper virus contains the sequence encoding the lost gene product, or “complementation element.” The complementation element should be expressible and stably integrated in the host cell, transgenic animal or helper virus, and preferably would be subject to little or no risk for recombination with the genome of the defective poxvirus.

Viruses produced in the complementing cell line are capable of infecting non-complementing cells, and further are capable of high-level expression of early gene products. However, in the absence of the essential gene product, host shut-off, DNA replication, packaging, and production of infectious virus particles does not take place.

In particularly preferred embodiments described herein, selection of desired target gene products expressed in a complex library constructed in vaccinia virus is accomplished through coupling induction of expression of the complementation element to expression of the desired target gene product. Since the complementation element is only expressed in those host cells expressing the desired gene product, only those host cells will produce infectious virus which is easily recovered.

In another preferred aspect, inactivation of the library constructed in a eukaryotic virus vector is carried out by treating a sample of the library constructed in a virus vector with 4′-aminomethyl-trioxsalen (psoralen) and then exposing the virus vector to ultraviolet (UV) light. Psoralen and UV inactivation of viruses is well known to those of ordinary skill in the art. See, e.g., Tsung, K. et al., J. Virol. 70:165-171 (1996), which is incorporated herein by reference in its entirety.

Psoralen treatment typically comprises incubating a cell-free sample of the virus vector with a concentration of psoralen ranging from about 0.1 μg/ml to about 20 μg/ml, preferably about 1 μg/ml to about 17.5 μg/ml, about 2.5 μg/ml to about 15 μg/ml, about 5 μg/ml to about 12.5 μg/ml, about 7.5 μg/ml to about 12.5 μg/ml, or about 9 μg/ml to about 11 μg/ml. Accordingly, the concentration of psoralen may be about 0.1 μg/ml, 0.5 μg/ml,1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. Preferably, the concentration of psoralen is about 10 μg/ml. As used herein, the term “about” takes into account that measurements of time, chemical concentration, temperature, pH, and other factors typically measured in a laboratory or production facility are never exact, and may vary by a given amount based on the type of measurement and the instrumentation used to make the measurement.

The incubation with psoralen is typically carried out for a period of time prior to UV exposure. This time period preferably ranges from about one minute to about 20 minutes prior to the UV exposure. Preferably, the time period ranges from about 2 minutes to about 19 minutes, from about 3 minutes to about 18 minutes, from about 4 minutes to about 17 minutes, from about 5 minutes to about 16 minutes, from about 6 minutes to about 15 minutes, from about 7 minutes to about 14 minutes, from about 8 minutes to about 13 minutes, or from about 9 minutes to about 12 minutes. Accordingly, the incubation time may be about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. More preferably, the incubation is carried out for 10 minutes prior to the UV exposure.

The psoralen-treated viruses are then exposed to UV light. The UV may be of any wavelength, but is preferably long-wave UV light, e.g., about 365 nm. Exposure to UV is carried out for a time period ranging from about 0.1 minute to about 20 minutes. Preferably, the time period ranges from about 0.2 minute to about 19 minutes, from about 0.3 minute to about 18 minutes, from about 0.4 minute to about 17 minutes, from about 0.5 minute to about 16 minutes, from about 0.6 minute to about 15 minutes, from about 0.7 minute to about 14 minutes, from about 8 minute to about 13 minutes, from about 0.9 minute to about 12 minutes from about 1 minute to about 11 minutes, from about 2 minutes to about 10 minutes, from about 2.5 minutes to about 9 minutes, from about 3 minutes to about 8 minutes, from about 4 minutes to about 7 minutes, or from about 4.5 minutes to about 6 minutes. Accordingly, the incubation time may be about 0.1 minute, about 0.5 minute, about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. More preferably, the virus vector is exposed to UV light for a period of about 5 minutes.

The preferred embodiments relating to vaccinia virus may be modified in ways apparent to one of ordinary skill in the art for use with any poxvirus vector. Vectors other than poxvirus or vaccinia virus may be used.

### 5.3 The Tri-Molecular Recombination Method

Traditionally, poxvirus vectors such as vaccinia virus have not been used to identify previously unknown genes of interest from a complex libraries because a high efficiency, high titer-producing method of constructing and screening libraries did not exist for vaccinia. The standard methods of heterologous protein expression in vaccinia virus involve in vivo homologous recombination and in vitro direct ligation. Using homologous recombination, the efficiency of recombinant virus production is in the range of approximately 0.1% or less. Although efficiency of recombinant virus production using direct ligation is higher, the resulting titer is relatively low. Thus, the use of vaccinia virus vector has been limited to the cloning of previously isolated DNA for the purposes of protein expression and vaccine development.

Tri-molecular recombination, as disclosed in Zauderer, PCT Publication No. WO 00/028016, is a novel, high efficiency, high titer-producing method for cloning in vaccinia virus. Using the tri-molecular recombination method, it is possible to generate recombinant viruses at efficiencies of at least 90%, and titers at least at least 2 orders of magnitude higher than those obtained by direct ligation.

Thus, in a preferred embodiment, libraries of polynucleotides capable of expressing regulator molecules are constructed in poxvirus vectors, preferably vaccinia virus vectors, by tri-molecular recombination.

By “tri-molecular recombination” or a “tri-molecular recombination method” is meant a method of producing a virus genome, preferably a poxvirus genome, and even more preferably a vaccinia virus genome comprising a heterologous insert DNA, by introducing two nonhomologous fragments of a virus genome and a transfer vector or transfer DNA containing insert DNA into a recipient cell, and allowing the three DNA molecules to recombine in vivo. As a result of the recombination, a viable virus genome molecule is produced which comprises each of the two genome fragments and the insert DNA.

Thus, the tri-molecular recombination method as applied to the present invention comprises: (a) cleaving an isolated virus genome, preferably a DNA virus genome, more preferably a linear DNA virus genome, and even more preferably a poxvirus or vaccinia virus genome, to produce a first viral fragment and a second viral fragment, where the first viral fragment is nonhomologous with the second viral fragment; (b) providing a population of transfer plasmids comprising polynucleotides which encode regulator molecules through operable association with a transcription control region, flanked by a 5′ flanking region and a 3′ flanking region, wherein the 5′ flanking region is homologous to said the viral fragment described in (a), and the 3′ flanking region is homologous to said second viral fragment described in (a); and where the transfer plasmids are capable of homologous recombination with the first and second viral fragments such that a viable virus genome is formed; (c) introducing the transfer plasmids described in (b) and the first and second viral fragments described in (a) into a host cell under conditions where a transfer plasmid and the two viral fragments undergo in vivo homologous recombination, i.e., trimolecular recombination, thereby producing a viable modified virus genome comprising a polynucleotide which encodes a regulator molecule and (d) recovering modified virus genomes produced by this technique. Preferably, the recovered modified virus genome is packaged in an infectious viral particle.

By “recombination efficiency” or “efficiency of recombinant virus production” is meant the ratio of recombinant virus to total virus produced during the generation of virus libraries of the present invention. As shown in Example 1, the efficiency may be calculated by dividing the titer of recombinant virus by the titer of total virus and multiplying by 100%. For example, the titer is determined by plaque assay of crude virus stock on appropriate cells either with selection (e.g., for recombinant virus) or without selection (e.g., for recombinant virus plus wild type virus). Methods of selection, particularly if heterologous polynucleotides are inserted into the viral thymidine kinase (tk) locus, are well-known in the art and include resistance to bromdeoxyuridine (BDUR) or other nucleotide analogs due to disruption of the tk gene. Examples of selection methods are described herein.

By “high efficiency recombination” is meant a recombination efficiency of at least 1%, and more preferably a recombination efficiency of at least about 2%, 2.5%, 3%, 3.5%, 4%, 5%, 10%, 20%, 30%, 40%, 50%, 60%, 70%, 75%, 80%, 85%, 90%, 95%, or 99%.

A number of selection systems may be used, including but not limited to the thymidine kinase such as herpes simplex virus thymidine kinase (Wigler, et al., Cell 11:223 (1977)), hypoxanthine-guanine phosphoribosyltransferase (Szybalska and Szybalski, Proc. Natl. Acad. Sci. USA 48:2026 (1962)), and adenine phosphoribosyltransferase (Lowy et al., Cell22:817 (1980)) genes which can be employed in tk⁻, hgprt₋ or aprt⁻ cells, respectively. Also, antimetabolite resistance can be used as the basis of selection for the following genes: dhfr, which confers resistance to methotrexate (Wigler et al., Natl. Acad. Sci. USA 77:3567 (1980); O'Hare et al., Proc. Natl. Acad. Sci. USA 78:1527 (1981)); gpt, which confers resistance to mycophenolic acid (Mulligan and Berg, Proc. Natl. Acad. Sci. USA 78:2072 (1981)); neo, which confers resistance to the aminoglycoside G-418 (Colberre-Garapin, et al., J. Mol. Biol. 150:1 (1981)); and hygro, which confers resistance to hygromycin (Santerre, et al., Gene 30:147 (1984)).

Together, the first and second viral fragments or “arms” of the virus genome, as described above, preferably contain all the genes necessary for viral replication and for production of infectious viral particles. Examples of suitable arms and methods for their production using vaccinia virus vectors are disclosed herein. See also Falkner et al., U.S. Pat. No. 5,770,212 for guidance concerning essential regions for vaccinia replication.However, naked poxvirus genomic DNAs such as vaccinia virus genomes cannot produce infectious progeny without virus-encoded protein protein(s)/function(s) associated with the incoming viral particle. The required virus-encoded functions,include an RNA polymerase that recognizes the transfected vaccinia DNA as a template, initiates transcription and, ultimately, replication of the transfected DNA. See Dorner, et al. U.S. Pat. No. 5,445,953.

Thus, o produce infectious progeny virus by trimolecular recombination using a poxvirus such as vaccinia virus, the recipient cell preferably contains packaging function. The ackaging function may be provided by helper virus, i.e., a virus that, together with the transfected naked genomic DNA, provides appropriate proteins and factors necessary for replication and assembly of progeny virus.The helper virus may be a closely related virus, for instance, a poxvirus of the same poxvirus subfamily as vaccinia, whether from the same or a different genus. In such a case it is advantageous to select a helper virus which provides an RNA polymerase that recognizes the transfected DNA as a template and thereby serves to initiate transcription and, ultimately, replication of the transfected DNA. If a closely related virus is used as a helper virus, it is advantageous that it be attenuated such that formation of infectious virus will be impaired. For example, a temperature sensitive helper virus may be used at the non-permissive temperature. Preferably, a heterologous helper virus is used. Examples include, but are not limited to a avipox virus such as fowlpox virus, or an ectromelia virus (mouse pox) virus. In particular, avipoxviruses are preferred, in that they provide the necessary helper functions, but do not replicate, or produce infectious virions in mammalian cells (Scheiflinger, et al, Proc. Natl. Acad. Sci. USA 89:9977-9981 (1992)). Use of heterologous viruses minimizes recombination events between the helper virus genome and the transfected genome which take place when homologous sequences of closely related viruses are present in one cell. See Fenner and Comben, Virology 5:530 (1958); Fenner, Virology 8:499 (1959).

Alternatively, the necessary helper functions in the recipient cell is supplied by a genetic element other than a helper virus. For example, a host cell can be transformed to produce the helper functions constitutively, or the host cell can be transiently transfected with a plasmid expressing the helper functions, infected with a retrovirus expressing the helper functions, or provided with any other expression vector suitable for expressing the required helper virus function. See Domer, et al. U.S. Pat. No 5,445,953.According to the trimolecular recombination method, the first and second viral genomic fragments are unable to ligate or recombine with each other, i.e., they do not contain compatible cohesive ends or homologous regions, or alternatively, cohesive ends have been treated with a dephosphorylating enzyme. In a preferred embodiment, a virus genome comprises a first recognition site for a first restriction endonuclease and a second recognition site for a second restriction endonuclease, and the first and second viral fragments are produced by digesting the viral genome with the appropriate restriction endonucleases to produce the viral “arms,” and the first and second viral fragments are isolated by standard methods. Ideally, the first and second restriction endonuclease recognition sites are unique in the viral genome, or alternatively, cleavage with the two restriction endonucleases results in viral “arms” which include the genes for all essential functions, i.e., where the first and second recognition sites are physically arranged in the viral genome such that the region extending between the first and second viral fragments is not essential for virus infectivity.

In a preferred embodiment where a vaccinia virus vector is used in the trimolecular recombination method, a vaccinia virus vector comprising a virus genome with two unique restriction sites within the tk gene is used. In certain preferred vaccinia virus genomes, the first restriction enzyme is NotI, having the recognition site GCGGCCGC in the tk gene, and the second restriction enzyme is ApaI, having the recognition site GGGCCC in the tk gene. Even more preferred are vaccinia virus vectors comprising a v7.5/tk virus genome or a vEL/tk virus genome.

According to this embodiment, a transfer plasmid with flanking regions capable of homologous recombination with the region of the vaccinia virus genome containing the thymidine kinase gene is used. A fragment of the vaccinia virus genome comprising the HindIII-J fragment, which contains the tk gene, is conveniently used.

Where the virus vector is a poxvirus, the insert polynucleotides are preferably operably associated with poxvirus expression control sequences, more preferably, strong constitutive poxvirus promoters such as p7.5 or a synthetic early/late promoter.

Accordingly, atransfer plasmid of the present invention comprises a polynucleotide encoding a regulator molecule through operable association with a vaccinia virus p7.5 promoter, or a synthetic early/late promoter.

By “insert DNA” is meant one or more heterologous DNA segments to be expressed in the recombinant virus vector. According to the present invention, “insert DNAs” are polynucleotides which encode regulator molecules. A DNA segment may be naturally occurring, non naturally occurring, synthetic, or a combination thereof. Methods of producing insert DNAs of the present invention are disclosed herein.

By “transfer plasmid” is meant a plasmid vector containing an insert DNA positioned between a 5′ flanking region and a 3′ flanking region as described above. The 5′ flanking region shares homology with the first viral fragment, and the 3′ flanking region shares homology with the second viral fragment. Preferably, the transfer plasmid contains a suitable promoter, such as a strong, constitutive vaccinia promoter where the virus vector is a poxvirus, upstream of the insert DNA. The term “vector” means a polynucleotide construct containing a heterologous polynucleotide segment, which is capable of effecting transfer of that polynucleotide segment into a suitable host cell. Preferably the polynucleotide contained in the vector is operably linked to a suitable control sequence capable of effecting the expression of the polynucleotide in a suitable host. Such control sequences include a promoter to effect transcription, an optional operator sequence to control such transcription, a sequence encoding suitable mRNA ribosome binding sites, and sequences which control the termination of transcription and translation. As used herein, a vector may be a plasmid, a phage particle, a virus, a messenger RNA, or simply a potential genomic insert. Once transformed into a suitable host, the vector may replicate and function independently of the host genome, or may in some instances, integrate into the genome itself. Typical plasmid expression vectors for mammalian cell culture expression, for example, are based on pRK5 (EP 307,247), pSV16B (WO91/08291) and pVL1392 (Pharmingen).

However, “a transfer plasmid,” as used herein, is not limited to a specific plasmid or vector. Any DNA segment in circular or linear or other suitable form may act as a vehicle for transferring the DNA insert into a host cell along with the first and second viral “arms” in the tri-molecular recombination method. Other suitable vectors include lambda phage, mRNA, DNA fragments, etc., as described herein or otherwise known in the art. A plurality of plasmids may be a “primary library” such as those described herein for lambda.

### 5.4 Additional Modifications of Trimolecular Recombination

Trimolecular recombination can be used to construct cDNA libraries in vaccinia virus with titers of the order of about 10⁷ pfu. There are several factors that limit the complexity of these cDNA libraries or other libraries. These include: the size of the primary cDNA library or other library, such as a library of polynucleotides encoding regulator molecules, that can be constructed in a plasmid vector, and the labor involved in the purification of large quantities (hundreds of micrograms) of virus “arms,” preferably vaccinia virus “arms” or other poxvirus “arms.” Modifications of trimolecular recombination that would allow for vaccinia or other virus DNA recombination with primary cDNA libraries or other libraries, such as polynucleotides encoding egulator molecules constructed in bacteriophage lambda or DNA or phagemids derived therefrom, or that would allow separate virus DNA arms to be generated in vivo following infection with a modified viral vector could greatly increase the quality and titer of the eukaryotic virus cDNA libraries or other libraries that are constructed using these methods.

### Transfer of cDNA inserts from a Bacteriophage Lambda Library to Vaccinia Virus

Lambda phage vectors have several advantages over plasmid vectors for construction of cDNA libraries or other libraries such as polynucleotides encoding regulator molecules. Plasmid cDNA (or other DNA insert) libraries or linear DNA libraries are introduced into bacteria cells by chemical/heat shock transformation, or by electroporation. Bacteria cells preferentially take up smaller plasmids, which potentially results in a decreased representation of longer cDNAs or other insert DNA, such as polynucleotides encoding egulator molecules,in a library. In addition, transformation can be a relatively inefficient process for introducing foreign DNA or other DNA into a cell and may require the use of commercially prepared competent bacteria in order to construct a cDNA library or other library, such as polynucleotides encoding regulator molecules. In contrast, lambda phage vectors can tolerate cDNA inserts of 12 kilobases or more without any size bias. Lambda vectors are packaged into virions in vitro using high efficiency commercially available packaging extracts so that the recombinant lambda genomes can be introduced into bacterial cells by infection. This results in primary libraries with higher titers and better representation of large cDNAs or other insert DNA, such as polynucleotides encoding egulator molecules,than is commonly obtained in plasmid libraries.

To enable transfer of cDNA inserts or other insert DNA, such as polynucleotides encoding regulator molecules, from a library constructed in a lambda vector to a eukaryotic virus vector such as vaccinia virus, the lambda vector is modified to include vaccinia virus DNA sequences that allow for homologous recombination with the vaccinia virus DNA. The following example uses vaccinia virus homologous sequences, but other viruses may be similarly used. For example, the vaccinia virus HindIII J fragment (comprising the vaccinia tk gene) contained in plasmid p7.5/ATG0/tk (as described in Example 1, infra) can be excised using HindIII and SnaBI (3 kb of vaccinia DNA sequence), and subcloned into the HindIII/SnaBI sites of pT7Blue3 (Novagen cat no. 70025-3) creating pT7B3.Vtk. The vaccinia tk gene can be excised from this vector with SacI and SnaBI and inserted into the SacI/SmaI sites of Lambda Zap Express (Stratagene) to create lambda.Vtk. The lambda.Vtk vector will contain unique NotI, BamHI, SmaI, and SalI sites for insertion of cDNA downstream of the vaccinia 7.5k promoter. cDNA libraries can be constructed in lambda.Vtk employing methods that are well known in the art.

DNA from a cDNA library or other library, such as polynucleotides encoding egulator molecules,constructed in lambda.Vtk, or any similar bacteriophage that includes cDNA inserts or other insert DNA with flanking vaccinia DNA sequences to promote homologous recombination, can be employed to generate cDNA or other insert DNA recombinant vaccinia virus. Methods are well known in the art for excising a plasmid from the lambda genome by coinfection with a helper phage (ExAssist phage, Stratagene cat no. 211203). Mass excision from a lambda based library creates an equivalent cDNA library or other library in a plasmid vector. Plasmids excised from, for example, the lambda.Vtk cDNA library will contain the vaccinia tk sequences flanking the cDNA inserts or other insert DNAs, such as regulator molecules. This plasmid DNA can then be used to construct vaccinia recombinants by trimolecular recombination. Another embodiment of this method is to purify the lambda DNA directly from the initial lambda.Vtk library, and to transfect this recombinant viral (lambda) DNA or fragments thereof together with the two large vaccinia virus DNA fragments for trimolecular recombination.

### Generation of Vaccinia Arms in Vivo

Purification and transfection of vaccinia DNA or other virus DNA “arms” or fragments is a limiting factor in the construction of polynucleotide libraries by trimolecular recombination. Modifications to the method to allow for the requisite generation of virus arms, in particular vaccinia virus arms, in vivo would allow for more efficient construction of libraries in eukaryotic viruses.

Host cells can be modified to express a restriction endonuclease that recognizes a unique site introduced into a virus vector genome. When a vaccinia virus infects these host cells, the restriction endonuclease will digest the vaccinia DNA, and will generate “arms” that can only be repaired, i.e., rejoined, by trimolecular recombination. Examples of restriction endonucleases include the bacterial enzymes NotI and ApaI, the Yeast endonuclease VDE (Hirata, R. et al, J. Biological Chemistry 265:6726-6733 (1990)), the Chlamydomonas eugametos endonuclease I-CeuI and others well-known in the art. For example, a vaccinia strain containing unique NotI and ApaI sites in the tk gene has been constructed, and a strain containing unique VDE and/or I-CeuI sites in the tk gene could be readily constructed by methods known in the art.

Constitutive expression of a restriction endonuclease would be lethal to a cell, due to the fragmentation of the chromosomal DNA by that enzyme. To avoid this complication, in one embodiment host cells are modified to express the gene(s) for the restriction endonuclease(s) under the control of an inducible promoter.

A preferred method for inducible expression utilizes the Tet-On Gene Expression System (Clontech). In this system expression of the gene encoding the endonuclease is silent in the absence of an inducer (tetracycline). This makes it possible to isolate a stably transfected cell line that can be induced to express a toxic gene, i.e., the endonuclease (Gossen, M. et al., Science 268:1766-1769 (1995)). The addition of the tetracycline derivative doxycycline induces expression of the endonuclease. In a preferred embodiment, BSC 1 host cells will be stably transfected with the Tet-On vector controlling expression of the NotI gene. Confluent monolayers of these cells will be induced with doxycycline and then infected with v7.5/tk (unique NotI site in tk gene), and transfected with cDNA or insert DNA recombinant transfer plasmids or transfer DNA or lambda phage or phagemid DNA. Digestion of exposed vaccinia DNA at the unique NotI site, for example, in the tk gene or other sequence by the NotI endonuclease encoded in the host cells produces two large vaccinia DNA fragments which can give rise to full-length viral DNA only by undergoing trimolecular recombination with the transfer plasmid or phage DNA. Digestion of host cell chromosomal DNA by NotI is not expected to prevent production of modified infectious viruses because the host cells are not required to proliferate during viral replication and virion assembly.

In another embodiment of this method to generate virus arms such as vaccinia arms in vivo, a modified vaccinia strain is constructed that contains a unique endonuclease site in the tk gene or other non-essential gene, and also contains a heterologous polynucleotide encoding the endonuclease under the control of the T7 bacteriophage promoter at another non-essential site in the vaccinia genome. Infection of cells that express the T7 RNA polymerase would result in expression of the endonuclease, and subsequent digestion of the vaccinia DNA by this enzyme. In a preferred embodiment, the v7.5/tk strain of vaccinia is modified by insertion of a cassette containing the cDNA encoding NotI with expression controlled by the T7 promoter into the HindIII C or F region (Coupar, E. H. B. et al, Gene 68:1-10 (1988); Flexner, C. et al., Nature 330:259-262 (1987)), generating v7.5/tk/T7NotI. A cell line is stably transfected with the cDNA encoding the T7 RNA polymerase under the control of a mammalian promoter as described ( Elroy-Stein, O. and Moss, B., Proc. Natl. Acad. Sci. USA 87:6743-6747(1990)). Infection of this packaging cell line with v7.5/tk/T7NotI will result in T7 RNA polymerase dependent expression of NotI, and subsequent digestion of the vaccinia DNA into arms. Infectious full-length viral DNA can only be reconstituted and packaged from the digested vaccinia DNA arms following trimolecular recombination with a transfer plasmid or phage DNA. In yet another embodiment of this method, the T7 RNA polymerase can be provided by co-infection with a T7 RNA polymerase recombinant helper virus, such as fowlpox virus (Britton, P. et al., J. General Virology 77:963-967 (1996)).

A unique feature of trimolecular recombination employing these various strategies for generation of large virus DNA fragments, preferably vaccinia DNA fragments in vivo is that digestion of the vaccinia DNA may, but does not need to precede recombination. It suffices that only recombinant virus escapes destruction by digestion. This contrasts with trimolecular recombination employing transfection of vaccinia DNA digested in vitro where, of necessity, vaccinia DNA fragments are created prior to recombination. It is possible that the opportunity for bimolecular recombination prior to digestion will yield a greater frequency of recombinants than can be obtained through trimolecular recombination following digestion.

### 5.5 Construction of MVA Trimolecular Recombination Vectors

In order to construct a Modified Vaccinia Ankara (MVA) vector suitable for trimolecular recombination, two unique restriction endonuclease sites must be inserted into the MVA tk gene. The complete MVA genome sequence is known (GenBank U94848). A search of this sequence revealed that restriction endonucleases AscI, RsrII, SfiI, and XmaI do not cut the MVA genome. Restriction endonucleases AscI and XmaI have been selected due to the commercial availability of the enzymes, and the size of the recognition sequences, 8 bp and 6 bp for AscI and XmaI respectively. In order to introduce these sites into the MVA tk gene a construct will be made that contains a reporter gene (E. coli gusA) flanked by XmaI and AscI sites. The Gus gene is available in pCRII.Gus (Merchlinsky, M. et al., Virology 238:444-451 (1997)). This reporter gene construct will be cloned into a transfer plasmid containing vaccinia tk DNA flanks and the early/late 7.5 k promoter to control expression of the reporter gene. The Gus gene will be PCR amplified from this construct using Gus specific primers. Gus sense 5′ ATGTTACGTCCTGTAGAAACC 3′ (SEQ ID NO:83), and Gus Antisense 5′TCATTGTTTGCCTCCCTGCTG 3′(SEQ ID NO:84). The Gus PCR product will then be PCR amplified with Gus specific primers that have been modified to include NotI and XmaI sites on the sense primer, and AscI and ApaI sites on the antisense primer. The sequence of these primers is: NX-Gus Sense 5′ AAAGCGGCCG CCCCGGGATG TTACGTCC 3′ (SEQ ID NO:85); and AA-Gus antisense 5′ AAAGGGCCCG GCGCGCCTCA TTGTTTGCC 3′ (SEQ ID NO:86). This PCR product will be digested with NotI and ApaI and cloned into the NotI and ApaI sites of p7.5/tk (Merchlinsky, M. et al., Virology 238:444-451 (1997)). The 7.5 k-XmaI-gusA-AscI construct will be introduced into MVA by conventional homologous recombination in permissive QT35 or BHK cells. Recombinant plaques will be selected by staining with the Gus substrate X-Glu (5-bromo-3 indoyl-β-D-glucuronic acid; Clontech) (Carroll, M. W. and Moss, B., Biotechniques 19:352-355 (1995)). MVA-Gus clones, which will also contain the unique XmaI and AscI sites, will be plaque purified to homogeneity. Large scale cultures of MVA-Gus will be amplified on BHK cells, and naked DNA will be isolated from purified virus. After digestion with XmaI and AscI the MVA-Gus DNA can be used for trimolecular recombination in order to construct cDNA expression libraries in MVA.

MVA is unable to complete its life cycle in most mammalian cells. This attenuation can result in a prolonged period of high levels of expression of recombinant cDNAs, but viable MVA cannot be recovered from infected cells. The inability to recover viable MVA from selected cells would prevent the repeated cycles of selection required to isolate functional cDNA recombinants of interest. A solution to this problem is to infect MVA infected cells with a helper virus that can complement the host range defects of MVA. This helper virus can provide the gene product(s) which MVA lacks that are essential for completion of its life cycle. It is unlikely that another host range restricted helper virus, such as fowlpox, would be able to complement the MVA defect(s), as these viruses are also restricted in mammalian cells. Wild type strains of vaccinia virus would be able to complement MVA. In this case however, production of replication competent vaccinia virus would complicate additional cycles of selection and isolation of recombinant MVA clones. A conditionally defective vaccinia virus could be used which could provide the helper function needed to recover viable MVA from mammalian cells under nonpermissive conditions, without the generation of replication competent virus.

The vaccinia D4R open reading frame (orf) encodes a uracil DNA glycosylase enzyme. This enzyme is essential for vaccinia virus replication, is expressed early after infection (before DNA replication), and disruption of this gene is lethal to vaccinia. It has been demonstrated that a stably transfected mammalian cell line expressing the vaccinia D4R gene was able to complement a D4R deficient vaccinia virus (Holzer, G. W. and Falkner, F. G., J. Virology 71:4997-5002 (1997)). A D4R deficient vaccinia virus would be an excellent candidate as a helper virus to complement MVA in mammalian cells.

In order to construct a D4R complementing cell line the D4R orf will be cloned from vaccinia strain v7.5/tk by PCR amplification using primers D4R-Sense 5′ AAAGGATCCA TAATGAATTC AGTGACTGTA TCACACG 3+ (SEQ ID NO:87), and D4R Antisense 5′ CTTGCGGCCG CTTAATAAAT AAACCCTTGA GCCC 3′(SEQ ID NO:88). The sense primer has been modified to include a BamHI site, and the anti-sense primer has been modified to include a NotI site. Following PCR amplification and digestion with BamHI and NotI the D4R orf will be cloned into the BamHI and NotI sites of pIRESHyg (Clontech). This mammalian expression vector contains the strong CMV Immediate Early promoter/Enhancer and the ECMV internal ribosome entry site (IRES). The D4RIRESHyg construct will be transfected into BSC1 cells and transfected clones will be selected with hygromycin. The IRES allows for efficient translation of a polycistronic mRNA that contains the D4Rorf at the 5′ end, and the Hygromycin phosphotransferase gene at the 3′ end. This results in a high frequency of Hygromycin resistant clones being functional (the clones express D4R). BSC1 cells that express D4R (BSC1.D4R) will be able to complement D4R deficient vaccinia, allowing for generation and propagation of this defective strain.

To construct D4R deficient vaccinia, the D4R orf (position 100732 to 101388 in vaccinia genome) and 983 bp (540 0 end) and 610 bp (340 end) of flanking sequence will be PCR amplified from the vaccinia genome. Primers D4R Flank sense 5′ ATTGAGCTCT TAATACTTTT GTCGGGTAAC AGAG 3′ (SEQ ID NO:89), and D4R Flank antisense 5′ TTACTCGAGA GTGTCGCAAT TTGGATTTT 3′ (SEQ ID NO:90) contain a SacI (Sense) and XhoI (Antisense) site for cloning and will amplify position 99749 to 101998 of the vaccinia genome. This PCR product will be cloned into the SacI and XhoI sites of pBluescript II KS (Stratagene), generating pBS.D4R.Flank. The D4R gene contains a unique EcoRI site beginning at nucleotide position 3 of the 657 bp orf, and a unique PstI site beginning at nucleotide position 433 of the orf. Insertion of a Gus expression cassette into the EcoRI and PstI sites of D4R will remove most of the D4R coding sequence. A 7.5k promoter- Gus expression vector has been constructed (Merchlinsky, M. et al., Virology 238: 444-451 (1997)). The 7.5-Gus expression cassette will be isolated from this vector by PCR using primers 7.5 Gus Sense 5′ AAAGAATTCC TTTATTGTCA TCGGCCAAA (SEQ ID NO:91) and 7.5Gus antisense 5′ AATCTGCAGT CATTGTTTGC CTCCCTGCTG 3′ (SEQ ID NO:92). The 7.5Gus sense primer contains an EcoRI site and the 7.5Gus antisense primer contains a PstI site. Following PCR amplification the 7.5Gus molecule will be digested with EcoRI and PstI and inserted into the EcoRI and PstI sites in pBS.D4R.Flank, generating pBS.D4R⁻/7.5Gus⁺. D4R⁻/Gus⁺ vaccinia can be generated by conventional homologous recombination by transfecting the pBS.D4R⁻/7.5Gus⁺ construct into v7.5/tk infected BSC1.D4R cells. D4R⁻/Gus⁺ virus can be isolated by plaque purification on BSC1.D4R cells and staining with X-Glu. The D4R- virus can be used to complement and rescue the MVA genome in mammalian cells.

In a related embodiment, the MVA genome may be rescued in mammalian cells with other defective poxviruses, and also by a psoralen/UV-inactivated wild-type poxviruses. Psoralen/UV inactivation is discussed herein.

### 5.6 Construction and Use of D4R Trimolecular Recombination Vectors

Poxvirus infection can have a dramatic inhibitory effect on host cell protein and RNA synthesis. These effects on host gene expression could, under some conditions, interfere with the selection of specific poxvirus recombinants that have a defined physiological effect on the host cell. Some strains of vaccinia virus that are deficient in an essential early gene have been shown to have greatly reduced inhibitory effects on host cell protein synthesis. Therefore, production of recombinant cDNA libraries in a poxvirus vector that is deficient in an early gene function may be advantageous for selection of certain recombinants that depend on continued active expression of some host genes. Disruption of essential viral genes prevents viral replication. Replication defective strains of vaccinia are rescued by providing the missing function through transcomplementation, such as by a host cell-encoded or helper virus-encoded gene under the control of an inducible promoter.

Infection of a cell population with a poxvirus library constructed in a replication deficient strain should greatly attenuate the effects of infection on host cell signal transduction mechanisms, differentiation pathways, and transcriptional regulation. An additional and important benefit of this strategy is that expression of the essential gene under the control of a inducible promoter can itself be the means of selecting recombinant virus that directly or indirectly lead to activation of that target transcriptional regulatory region. Examples of inducible promoters include the promoter of a gene activated as a result of crosslinking surface immunoglobulin receptors on early B cell precursors or the promoter of a gene that encodes a marker induced following stem cell differentiation. Additional examples of inducible promoters include cell type-restricted promoters, tissue-restricted promoters, temporally-regulated promoters, spatially-regulated promoters, proliferation-induced promoters, cell-cycle specific promoters, etc., such as those described herein or well-known in the art. If such a promoter drives expression of an essential viral gene, then only those viral recombinants that directly or indirectly activate expression of the transcriptional regulator will replicate and be packaged as infectious particles. This method has the potential to give rise to much lower background then selection methods based on expression of dipA or a CTL target epitope because uninduced cells will contain no replication competent vaccinia virus that might be released through non-specific bystander effects. The selected recombinants can be further expanded in a complementing cell line or in the presence of a complementing helper virus or transfected plasmid.

A number of essential early vaccinia genes have been described. Preferably, a vaccinia strain deficient for the D4R gene could be employed. The vaccinia D4R open reading frame (orf) encodes a uracil DNA glycosylase enzyme. This enzyme is reqired for viral DNA replication and disruption of this gene is lethal to vaccinia (Millns, A. K. et al., Virology 198:504-513 (1994)). It has been demonstrated that a stably transfected mammalian cell line expressing the vaccinia D4R gene is able to complement a D4R deficient vaccinia virus (Holzer, G. W. and Falkner, F. G., J. Virology 71:4997-5002 (1997)). In the absence of D4R complementation, infection with the D4R deficient vaccinia results in greatly reduced inhibition of host cell protein synthesis (Holzer and Falkner). It has also been shown that a foreign gene inserted into the tk gene of D4R deficient vaccinia continues to be expressed at high levels, even in the absence of D4R complementation (Himly, M. et al., Protein Expression and Purification 14: 317-326 (1998)). The replication deficient D4R strain is, therefore, well-suited for selection of viral recombinants that depend on continued active expression of some host genes for their physiological effect.

To implement this strategy for selection of specific recombinants from, for example, representative cDNA libraries constructed in a D4R deficient vaccinia strain the following cell lines and vectors may be used:

1. D4R expressing complementing cell line is used for expansion of D4R deficient viral stocks.

2. The D4R gene is deleted or inactivated in a viral strain suitable for trimolecular recombination.

3. Plasmid or viral constructs are generated that express D4R under the control of an inducible promoter such as the promoter for expression of type X collagen, which is induced following induction of chondrocyte differentiation from C3H10T½ progenitor cells. Stable transfectants of these constructs in the relevant cell line are used to rescue specific recombinants. Alternatively, a helper virus expressing the relevant construct can be employed for induction in either cell lines or primary cultures.

### Construction of a D4R Complementing Cell Line

A D4R complementing cell line is constructed as follows. First, the D4R orf (position 100732 to 101388 in vaccinia genome) is cloned from vaccinia strain v7.5/tk by PCR amplification using the following primers: D4R-sense, 5′ AAAGAATTCA TAATGAATTC AGTGACTGTA TCACACG 3′, designated herein as SEQ ID NO:93; and D4R-antisense: 5′ CTTGGATCCT TAATAAATAA ACCCTTGAGC CC 3′, designated herein as SEQ ID NO:94.

The sense primer is modified to include an EcoRI site, and the anti-sense primer is modified to include a BamHI site (both underlined). Following standard PCR amplification and digestion with EcoRI and BamHI, the resulting D4R orf is cloned into the EcoRI and BamHI sites of pIRESneo (available from Clontech, Palo Alto, Calif.). This mammalian expression vector contains the strong CMV immediate early promoter/enhancer and the ECMV internal ribosome entry site (IRES). The D4R/IRESneo construct is transfected into BSC1 cells and transfected clones are selected with G418. The IRES allows for efficient translation of a polycistronic mRNA that contains the D4Rorf at the 5′ end, and the neomycin phosphotransferase gene at the 3′ end. This results in a high frequency of G418 resistant clones being functional (the clones express D4R). Transfected clones are tested by northern blot analysis using the D4R gene as probe in order to identify clones that express high levels of D4R mRNA. BSC1 cells that express D4R (BSC1.D4R) are able to complement D4R deficient vaccinia, allowing for generation and propagation of D4R defective viruses.

### Construction of D4R Deficient Vaccinia Vector

A D4R-deficient vaccinia virus, suitable for trimolecular recombination as described herein, is constructed by disruption of the D4R orf (position 100732 to 101388 in vaccinia genome) through the insertion of an E. coli Gus A expression cassette into a 300-bp deletion, by the following method.

In order to insert the GusA gene, regions flanking the insertion site are amplified from vaccinia virus as follows. The left flanking region is amplified with the following primers: D4R left flank sense: 5′AATAAGCTTT GACTCCAGAT ACATATGGA 3′, designated herein as SEQ ID NO:95; and D4R left flank antisense: 5′ AATCTGCAGC ACCAGTTCCA TCTTT 3′, designated herein as SEQ ID NO:96. These primers amplify a region extending from position 100167 to position 100960 of the vaccinia genome, and have been modified to include a HindIII (Sense) and PstI (Antisense) site for cloning (both underlined). The resulting PCR product is digested with HindIII and PstI, and cloned into the HindIII and PstI sites of pBS (available from Stratagene), generating pBS.D4R.LF. he right flanking region is amplified with the following primers: D4R right flank sense: 5′ AATGGATCCT CATCCAGCGG CTA 3′, designated herein as SEQ ID NO:97; and D4R right flank antisense: 5′ AATGAGCTCT AGTACCTACA ACCCGAA 3′, designated herein as SEQ ID NO:98. These primers amplify a region extending from position 101271 to position 101975 of the vaccinia genome, and have been modified to include a BamHI (Sense) and SacI (Antisense) site for cloning (both underlined). The resulting PCR product is digested with BamHI and SacI, and cloned into the BamHI and SacI sites of pBS.D4R.LF, creating pBS.D4R.LF/RF.

An expression cassette comprising the GusA coding region operably associated with a poxvirus synthetic early/late (E/L) promoter is inserted into pBS.D4R.LF/RF by the following method. The E/L promoter- Gus cassette is derived from the pEL/tk-Gus construct described in Merchlinsky, M. et al., Virology 238: 444-451 (1997). The NotI site immediately upstream of the Gus ATG start codon is removed by digestion of pEL/tk-Gus with NotI, followed by a fill in reaction with Klenow fragment and religation to itself, creating pEL/tk-Gus(NotI-). The E/L-Gus expression cassette is isolated from pEL/tk-Gus(NotI-) by standard PCR using the following primers: EL-Gus sense: 5′ AAAGTCGACG GCCAAAAATT GAAATTTT 3′, designated herein as SEQ ID NO:99; and EL-Gus antisense: 5′ AATGGATCCT CATTGTTTGC CTCCC 3′, designated herein as SEQ ID NO:100. The EL-Gus sense primer contains a SalI site and the EL-Gus antisense primer contains a BamHI site (both underlined). Following PCR amplification the EL-Gus cassette is digested with SalI and BamHI and inserted into the SalI and BamHI sites in pBS.D4R.LF/RF generating pBS.D4R-ELGus. This transfer plasmid contains an EL-Gus expression cassette flanked on both sides by D4R sequence. There is also a 300 bp deletion engineered into the D4R orf.

D4R⁻/Gus⁺ vaccinia viruses suitable for trimolecular recombination are generated by conventional homologous recombination following transfection of the pBS.D4R⁻/ELGus construct into v7.5/tk-infected BSC1.D4R cells. D4R⁻/Gus⁺ virus are isolated by plaque purification on BSC1.D4R cells and staining with X-Glu (Carroll, M. W. and Moss, B. Biotechniques 19:352-355 (1995)). This new strain is designated v7.5/tk/Gus/D4R.

DNA purified from v7.5/tk/Gus/D4R is used to construct, for example, representative cDNA libraries by the trimolecular recombination method using the BSC1.D4R complementing cell line.

### Preparation of Host Cells Expressing D4R Under the Control of Inducible Promoters

Host cells which express the D4R gene upon induction of an inducible promoter are prepared as follows. Plasmid constructs are generated that express the vaccinia D4R gene under the control of an inducible promoter. Examples of inducible promoters include, but are not limited to the promoter for a marker of differentiation, such as type X collagen. The vaccinia D4R orf is amplified by PCR using primers D4R sense and D4R antisense described above. These PCR primers are modified as needed to include desirable restriction endonuclease sites. The D4R orf is then cloned in a suitable eukaryotic expression vector (which allows for the selection of stably transformed cells) in operable association with any desired promoter employing methods known to those skilled in the art.

The D4R gene, in operable association with the inducible promoter such as the type X collagen promoter is stably transfected into a suitable cell line, for example, C3H110T½ progenitor cells. The resulting host cells are used in the selection, screening, or production of regulator molecules using libraries prepared in v7.5/tk/Gus/D4R. Differentiation results in the induction of expression of the D4R gene product. Expression of D4R complements the defect in the v7.5/tk/Gus/D4R genomes in which the libraries are produced, allowing the production of infectious virus particles.

### Example 6

### Construction of Random Peptide Libraries in a Scaffold Based on Fibronectin Type III Domain

### 6.1 Construction of p7.5/FN3/tk

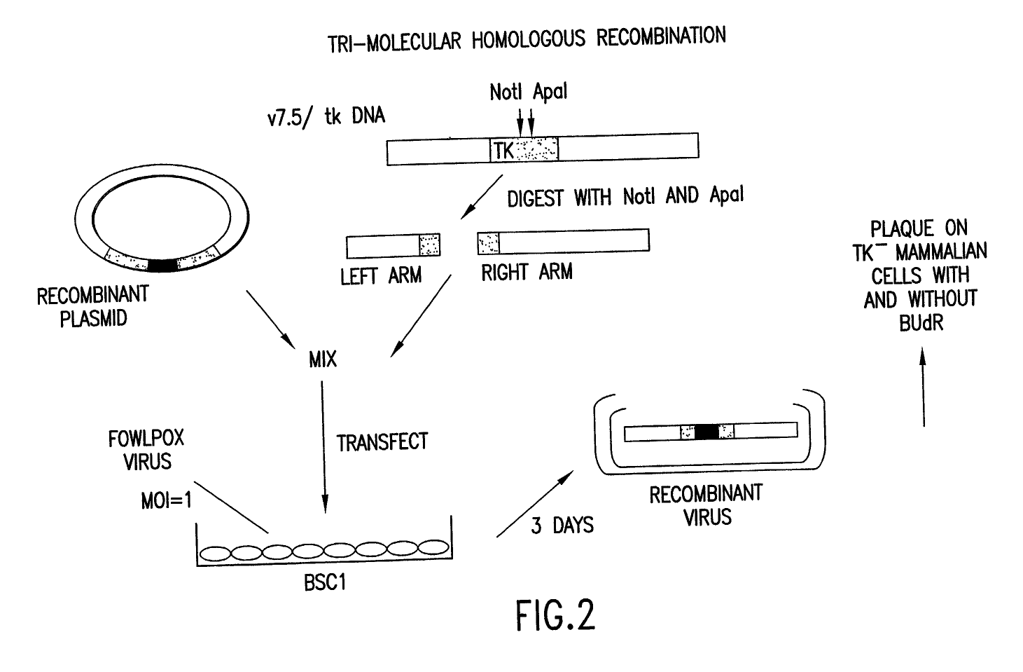

The Fibronectin (FN3) gene (Koide, A. et al., J. Mol. Bio. 284:1141-1151 (1998), which is incorporated by reference herein in its entirety) is subcloned into the vaccinia virus transfer plasmid p7.5/tk by the following method. Briefly, the FN3- pBluescript based phage display vector (Koide et al., FIG. 2) is digested with NdeI and the overhang filled in using pfu DNA Polymerase, creating a blunt 5′ end. The FN3 cDNA is excised from the phagemid vector by digestion with XhoI. Following gel purification the FN3 cDNA is inserted into the SmaI (blunt end site) and SalI (overhang is compatible with XhoI overhang) sites of p7.5/tk, prepared as described in Example 1, and in Zauderer, PCT Publication WO 00/028016 , creating p7.5/FN3/tk. This ligation disrupts both the SmaI and SalI sites in p7.5/FN3/tk.

A cDNA library encoding random peptide sequences in the scaffold of the Fibronectin type III domain is constructed in two stages. In the first stage, amino acids 29 (Val) and 30 (Thr), the second and third residues of the BC loop, are randomized as described below. This random library (p7.5/FN3-BC Random/tk) comprises 400 independent clones (20²). Plasmid DNA from this random library is purified. In the second stage, amino acids 78-80 (Arg, Gly, Asp), the second, third and fourth residues of the FG loop of FN3 in p7.5/FN3-BC Random/tk, are randomized and used to construct a second more diverse library, p7.5/FN3-BC/FG Random/tk). This library is constructed to contain a minimum of 3×10⁶ (20²×20³) clones in order to ensure complete sampling of all combinations of randomized residues in both BC and FG loops.

### 6.2 Construction of p7.5/FN3-BC Random/tk Library

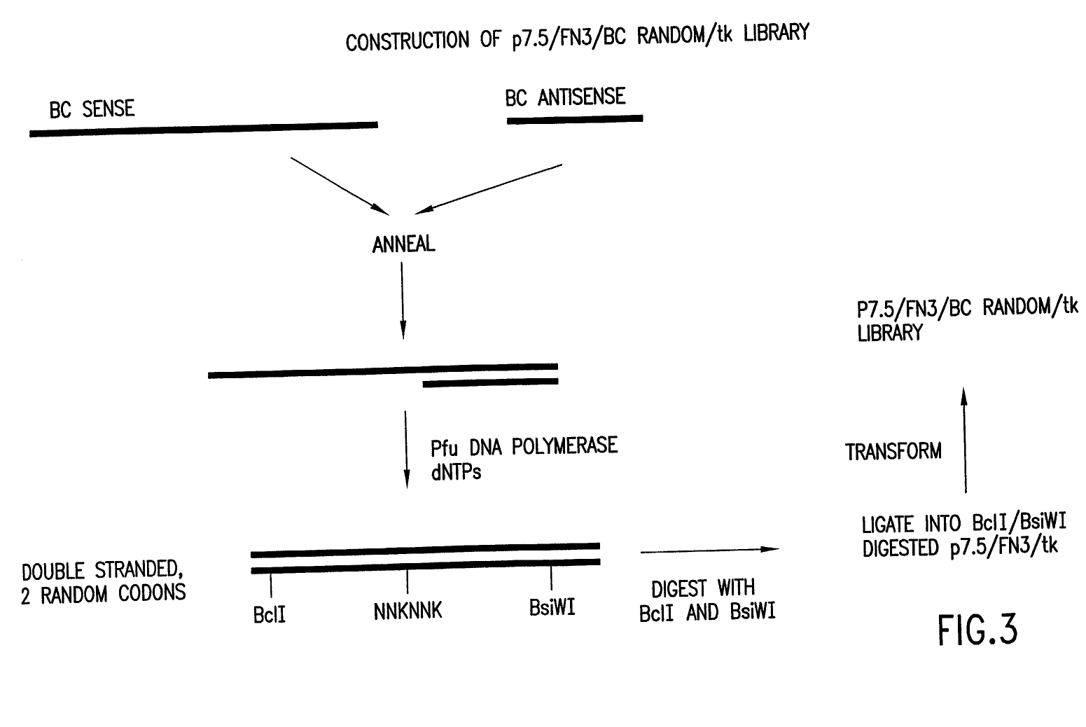

Amino acids 2 and 3 of the FN3 BC loop are randomized according to the following method. See FIG. 3 for a diagram of this cloning strategy. A single stranded oligonucleotide, BC Sense, encoding the BC Loop region of FN3 is synthesized. The sequence of this oligonucleotide, denoted herein as SEQ ID NO:101, is: 5′ AAAATGATCA GCTGGGATGC TCCTGCANNKN NKGTGCGTTA TTACCGTATC ACGTACGGTG A3′. N denotes an equimolar mixture of A, G, T, C. K denotes an eqimolar mixture of G and T. NNK can encode all 20 amino acids and one stop codon. Important features of this oligonucleotide are indicated in bold type and include: 5′ BclI site (TGATCA), 2 random codons (NNK), BsiWI site (CGTACG).

A synthetic oligonucleotide complementary to BC Sense is annealed to BC Sense. The sequence of this second oligonucleotide, BC Antisense, denoted herein as SEQ IDNO:102, is: 5′ TCACCGTACG TGATACGG3′. The region of BC Sense that BC Antisense anneals to is underlined above. After annealing BC Antisense to BC Sense, BC Sense is copied by the addition of a DNA Polymerase such as Pfu and all 4 dNTPs. This generates a double stranded DNA with a BclI site at the 5′ end, 2 random codons, and a BsiWI site at the 3′ end. This DNA is digested with BclI and BsiWI and ligated into the matching sites in p7.5/FN3/tk. The product of this ligation reaction is transformed into bacteria in order to generate the plasmid library p7.5/FN3/BC Random/tk. This library preferably contains more than 400 independent clones.

### 6.3 Construction of p7.5/FN3-BC/FG Random/tk

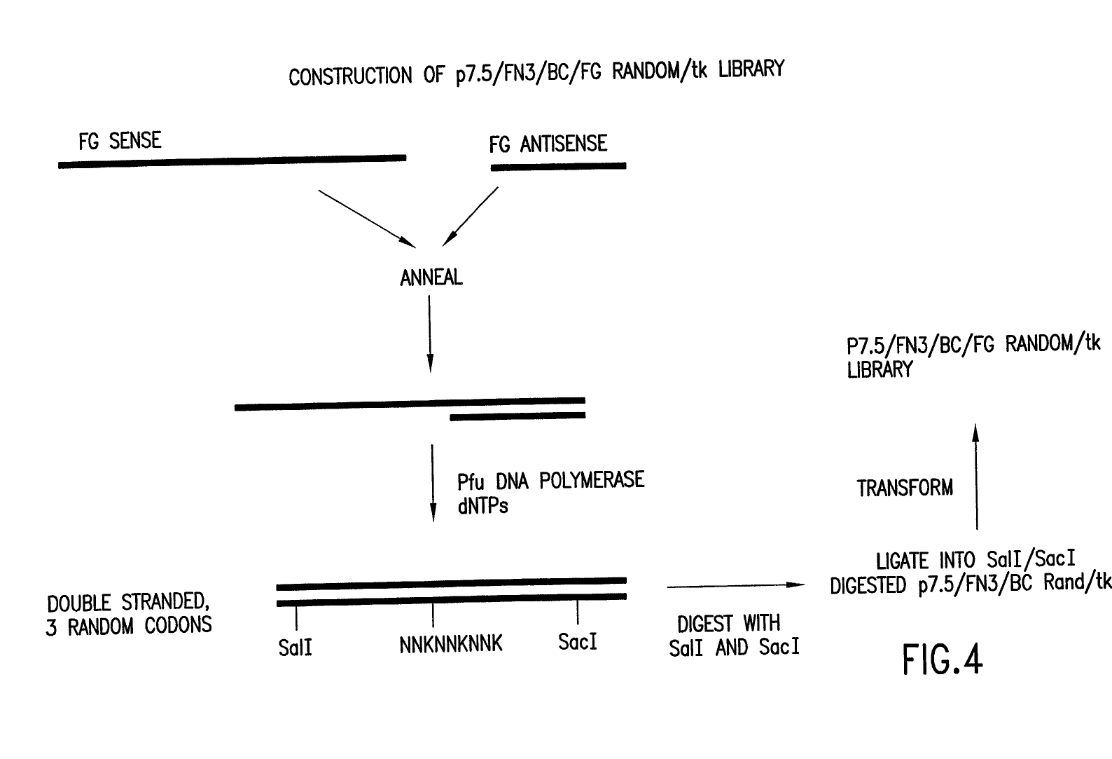

Amino acids 2-4 of the FN3 FG loop are randomized by the following method. See FIG. 4 for a diagram of this cloning strategy. A single stranded oligonucleotide, FG Sense, encoding these three randomized codons will be synthesized. The sequence of FG Sense, denoted herein as SEQ IDNO:103, is: 5′GGGTGTCGAC TATACCATCA CTGTATACGC TGTTACTGGC NNKNNKNNKA GCCCAGCGAG CTCCAAGCCA3′. N denotes an equimolar mixture of A, G, T, C, and K denotes an equimolar mixture of G and T. NNK can encode all 20 amino acids and one stop codon. Important features of this oligonucleotide are indicated in bold type and include: 5′ SalI site (GTCGAC), 3 random codons (NNK), SacI site (GAGCTC).

An oligonucleotide complementary to FG Sense is annealed to FG Sense. The sequence of this oligonucleotide, FG Antisense, denoted herein as SEQ ID NO:104, is:5′TGGCTTGGAG CTCGCTGG3′. The region of FG Sense that FG Antisense anneals to is underlined above. After annealing FG Antisense to FG Sense, FG Sense is copied by the addition of a DNA Polymerase such as Pfu and all 4 dNTPs. This generates double stranded DNA with a SalI site at the 5′ end, 3 random codons, and a SacI site at the 3′ end. This double stranded DNA is digested with SalI and SacI, and used as a pool of inserts to construct a random library. Plasmid DNA purified from the p7.5/FN3/BC Random/tk (BC Random library constructed above) is digested with SalI and SacI. The SalI and SacI digested FG Random oligonucleotides are ligated into SalI and SacI digested p7.5/FN3/BC Random/tk DNA, produced as described in section 6.2. These ligated molecules are transformed into bacteria in order to construct the library p7.5/FN3/BC/FG Random/tk. This library contains 2 random codons in the BC loop, and three random codons in the FG loop. This library preferably contains a minimum of 3×10⁶ (20²×20³) clones in order to ensure complete sampling of all combinations of randomized residues.

As described by Koide et al., a number of other modifications to the FN3 domain can contribute to stability of the scaffold and the peptide loops. These include substitution of threonine for arginine in the sixth position of the FN3 natural sequence to prevent thrombin cleavage at this residue. Also, residues 82-84, the 6^{th}, 7^{th}, and 8^{th} residues of the FG loop may be deleted to provide greater structural integrity to the incorporated peptides.

The p7.5/FN3/BC/FG Random/tk random peptide plasmid library is then transferred to a vaccinia virus based vector employing trimolecular recombination as described in Example 1. The same principles of construction are applicable to other poxvirus vectors.

### Example 7

### Identification of Regulatory Pathway Genes Involved in Muskuloskeletal Stem Cell Differentiation and Disease by Screening and/or Selection with Candidate Regulator Molecules using Suicide and Other Reporter Gene Constructs

Functionally mature and terminally differentiated cells of the musculoskeletal system, as defined by the expression of a specific gene product (a marker) that is only produced in those cells, are derived from stem cells. These stem cells are instructed to initiate the appropriate differentiation program by soluble factors, which initiate a signaling cascade that results in new gene expression. The products of new gene expression are directly involved in the cellular differentiation process. It has been demonstrated in other cell systems that the signal that normally initiates this differentiation process can be circumvented by introducing a downstream gene into the stem cell. Culture systems have been developed that reproduce the normal differentiation of chondrocytes, osteoblasts, and osteoclasts from progenitor cells. Appropriate markers are used to evaluate the authenticity and purity at various stages of differentiation.

Random regulator molecules, e.g., regulator polypeptides or regulator U1 SnRNAs are prepared in vaccinia virus as described herein. The libraries are used to infect a stem cell line which has been modified to contain a suicide gene construct such that if the differentiation program is initiated, the cell will die and release its recombinant virus. This virus, containing a polynucleotide which encodes a polypeptide or U1 SnRNA that regulates the differentiation program, can be readily recovered by washing, aspiration, etc., as described herein. To verify the regulatory function of the recovered target polynucleotides, the recovered polynucleotides are used to isolate the full-length regulatory cDNA which is then introduced into human primary stem cells, which can then be assessed for development into the appropriate lineage.

Combining trimolecular recombination, in vitro musculoskeletal cell differentiation, and direct selection and/or screening for regulatory molecules as described herein allows for the identification of genes that control growth and development. The genes identified are candidate pharmaceuticals or pharmiaceutical targets.

Stem cells. The genes that regulate differentiation of mature tissues from precursors or stem cells have been especially difficult to study because terminally differentiated cells often cease to proliferate. As a result it is in effect impossible to recover specific functional genes that induce differentiation following DNA transfection or retroviral transduction. It is, however, possible to design a system in which differentiation results in cell death. Under these conditions, genes that promote differentiation can be isolated from a vaccinia library that expresses cDNA of the differentiated cell type by “lethality based selection.” Every differentiated cell is distinguished from its precursors by expression of some specific gene product. Transcriptional activation of the promoter for that gene often serves as a surrogate marker of differentiation. If a construct of that specific promoter driving expression of a toxin such as the diphtheria A chain is transfected into a proliferating precursor, then any regulator molecule that directly or indirectly promotes differentiation will result in cell death. If that regulator molecule, e.g., a regulator polypeptide or a regulator U1 SnRNA, is introduced as a member of a random regulator molecule library produced in a vaccinia expression vector, then it can be readily recovered from dying differentiated cells. These methods are applicable to any stem cell population that can be induced to differentiate into a well-defined cell type or tissue. Stem cells have been described for a wide variety of tissues including but not limited to different types of blood cells, epidermal cells, neurons, glial cells, kidney cells, and liver cells. Among these also are the different stem cells of the musculoskeletal system including the precursors of chondrocytes, osteoblasts, osteoclasts, and myocytes.

Osteoclasts. Bone is the only organ that contains a cell type, the osteoclast, whose function is to destroy the organ in which it develops and resides. This destruction, or resorption, of bone occurs throughout life and in the healthy individual is counterbalanced by de novo bone formation in a processs called bone remodeling. The genetic control of osteoclast differentiation is one of the best understood examples of stem cell differentiation. The methods and strategies of this invention can be applied to identify genes that regulate stem cell differentiation just as they have been applied to identify the targets of immune cytotoxicity, as described in Zauderer, P.C.T. Publication No. WO 00/028016. This is illustrated specifically for the analysis of osteoclast differentiation.

Strategies are described to detect and isolate both genes that positively or negatively regulate differentiation including genes that are expressed in the differentiating cell itself or that are a secreted product of another producing cell that influences differentiation in a paracrine fashion. In all cases a cell type or cell line that can be induced to differentiate into mature osteoclasts in response to a specific signal, preferably RANK Ligand (RANKL), is employed to detect and isolate recombinant vaccinia virus expressing regulator molecules that regulate osteoclast differentiation. In a preferred embodiment, RAW cells are employed. RAW cells are a continuously growing murine myelomonocytic cell line that can be induced to differentiate into osteoclasts by treatment with a range of concentrations of RANK ligand (RANKL), preferably 10 ng/ml (Hsu, H. et al., Proc Natl Acad Sci USA 96(7):3540-45 (1999); Owens, J. M. et al., J. Cell Physiol 1 79:170 (1999)). These or similarly responsive cells are transfected with a suicide gene construct comprising a promoter that normally drives expression of a gene product that is recognized as a marker of fully differentiated osteoclasts but which is linked in this construct to expression of a suicide gene. In a preferred embodiment the promoter is that of the osteoclast differentiation marker TRAP and the suicide gene encodes the A chain of diphtheria toxin (TRAP/DT-A).

### 7.1 Detection and Isolation of Genes That Positively Regulate Differentiation

Regulator polypeptide-based strategy. A vaccinia library of random candidate regulator polypeptides, e.g., Fn3-based regulator polypeptides is constructed as described in Examples 5 and 6. RAW cells or other osteoclast progenitor cells that have been transfected with a TRAP/DT-A or similar suicide gene construct are infected with the vaccinia library, at a preferred multiplicity of infection (MOI) of between 0.1 and 10. Any vaccinia recombinant that expresses a regulator polypeptide that promotes differentiation to the mature TRAP expressing phenotype will result in synthesis of the toxin, and death of the infected cell. Such cells and their contents are released from the cell monolayer. Vaccinia virus recombinants extracted from the cells and cell contents released into the culture supernatant are enriched for the desired vaccinia recombinants. As described for selection of recombinants that encode cytotoxic target antigens described in Zauderer, P.C.T. Publication No. WO 00/028016, this selection process can be repeated through multiple cycles until the desired level of enrichment is achieved. Following isolation of a polynucleotide which encodes a suitable regulator polypeptide according to this method, the variable regions contained therein are used as a probe to isolate a full length cDNA encoding the native regulatory polypeptide of interest. TRAF6 (Lomaga, M. A. et al., Genes Dev 13:1015 (1999)), c-Fos (Wang, Z. Q. et al., Nature 360:741 (1992)), and c-Src (Soriano, P. et al., Cell 64:693 (1991)), are examples of positive regulators of osteoclast differentiation that could have been isolated through this method.

U1 SnRNA-based strategy. According to this method, a random library of sequences expressed in a U1 SnRNA scaffold is produced in vaccinia virus. Candidate regulator molecules in this library may inhibit processing of nascent nuclear transcripts, thereby suppressing expression of genes in a regulatory pathway of interest. To detect sequences required for differentiation, RAW cells or other progenitor cells transfected with TRAP/DT-A or similar suicide construct are treated with an agent that induces differentiation, in a preferred embodiment with 10 ng/ml RANKL. Under these conditions almost all transfectants differentiate and undergo suicide gene mediated cell death. Only cells that have been infected with a vaccinia recombinant that inhibits expression of an essential regulator of differentiation will survive and remain adherent. Virus extracted from the remaining adherent monolayer will, therefore, be enriched for sequences homologous to the desired positive regulators of differentiation. This selection process can also be repeated through several cycles until the desired degree of enrichment of recombinants in the adherent monolayer is achieved. The variable regions inserted in the U1 SnRNA scaffold are then isolated from the recovered vaccinia viruses, and are employed to select the actual full-length coding sequences of the regulatory genes of interest. TRAF6 (Lomaga, M. A. et al., Genes Dev 13:1015 (1999)), c-Fos (Wang, Z. Q. et al., Nature 360:741 (1992)), and c-Src (Soriano, P. et al., Cell 64:693 (1991)), are examples of positive regulators of osteoclast differentiation that could have been isolated through this method.

### 7.2 Detection and Isolation of Genes That Negatively Regulate Differentiation.

Regulator polypeptide-based strategy. A vaccinia library of random candidate regulator polypeptides, e.g., Fn3-based regulator polypeptides, constructed as described in Examples 5 and 6, is used to infect indicator cells transfected with TRAP/DT-A or similar suicide construct as described above. The indicator cells are treated with an agent that induces differentiation, preferably 10 ng/ml RANKL. Under these conditions almost all transfectants differentiate and undergo suicide gene mediated cell death. Only cells that are infected with a vaccinia recombinant expressing a regulator polypeptide that inhibits differentiation will survive and remain adherent. Virus extracted from the remaining adherent monolayer will, therefore, be enriched for sequences homologous to the desired negative regulators of differentiation. This selection process can be repeated through several cycles until the desired degree of enrichment of recombinants in the adherent monolayer is achieved. A negative intracellular regulator of osteoclast differentiation has not as yet been isolated. However, it has been suggested that the Est-1 transcription factor plays such a role in differentiation of B lymphocytes (Bories, J. C. et al., Nature 377(6550):635-8 (1995)).

U1 SnRNA-based strategy. According to this method, a random library of sequences expressed in a U1 SnRNA scaffold is produced in vaccinia virus. Candidate regulator molecules in this library may inhibit processing of nascent nuclear transcripts, thereby suppressing expression of genes in a regulatory pathway of interest. If the targeted sequence encodes an essential factor that suppresss cell differentiation, then in the absence of an effective inhibitory signal RAW cells or other progenitor cells transfected with TRAP/DT-A or similar suicide construct will either spontaneously differentiate or will differentiate in response to otherwise suboptimal signals. Differentiation to the mature TRAP expressing phenotype will result in synthesis of the toxin, and death of the infected cell. Such cells and their contents will be released from the cell monolayer. Vaccinia virus recombinants extracted from the cells and cell contents released into the culture supernatant are enriched for U1 SnRNAs possessing variable regions which suppress expression of negative regulators of differentiation. This selection process can be repeated through multiple cycles until the desired level of enrichment is achieved. The U1 SnRNAs obtained can be employed as a probe to isolate transcripts or cDNAs encoding the actual full-length coding sequence of the desired negative regulator molecule. A negative intracellular regulator of osteoclast differentiation has not as yet been isolated. However, it has been suggested that the Est-1 transcription factor plays such a role in differentiation of B lymphocytes (Bories, J. C. et al., Nature 377(6550):635-8 (1995)).

### 7.3 Detection and Isolation of Secreted Products That Regulate Differentiation

In another embodiment of the present invention, inserts are selected based on autocrine or paracrine activity. Thus, gene products such as proteins or peptides expressed in a host cells may function on that host cell after being secreted, or may function on a second cell after being secreted. Such second cell may be the same type of cell as the host cell or may be a different type of cell from the host cell. The secreted gene product may modulate differentiation, such as inducing or suppressing differentiation. If the gene to be identified and isolated functions only in paracrine fashion, that is being produced in one cell that affects activation or differentiation of a second cell, then the strategy of “lethality based” selection described in the previous paragraphs is not applicable since the expressing cell does not itself become non-viable or non-adherent. Nevertheless, as described below, the efficiency with which vaccinia recombinants can be introduced in a wide variety of cells and the high level of expression from replicating viral genomes is a great advantage for screening functional gene expression even where direct selection is not possible.

A vaccinia library of random candidate regulator polypeptides produced essentially as described in Examples 5 and 6, but comprising a molecular scaffold with “targeting sequences” allowing surface and/or extracellular expression of the candidate regulator polypeptides, is constructed. Producer cells are selected that do not either induce or suppress induction of differentiation of RAW cells or other osteoclast progenitors. These may include but are not limited to fibroblastoid or lymphoid cells and cell lines or RAW cells themselves. In a preferred embodiment, RAW cells are employed as an indicator target for differentiation. These or similarly responsive cells are transfected with an indicator gene construct comprising a promoter that normally drives expression of a gene product that is recognized as a marker of fully differentiated osteoclasts but which is linked in this construct to expression of an easily detected indicator gene product. In a preferred embodiment the promoter is that of the osteoclast differentiation marker TRAP and the indicator gene encodes the enzyme luciferase (TRAP/luciferase).

Multiple cultures of producer cells are separately infected with vaccinia virus libraries of regulator polypeptides expanded from a small initial pool, preferably an initial pool of between 1 and 1000 viral pfu is expanded to 10 to 10,000 pfu prior to infection of between 100 and 10,000 producer cells. Each pool of infected producer cells is cocultured with indicator cells that have been transfected with TRAP/luciferase or a similar indicator construct.

Secreted molecules that induce differentiation. Membrane expression or secretion of any candidate regulator polypeptide that promotes differentiation of the indicator cells to the mature TRAP expressing phenotype will result in synthesis of luciferase in those cells and, upon addition of luciferase assay reagents as is well known in the art, will give rise to a readily detectable signal from wells that express that recombinant gene product. Recombinant vaccinia viruses are extracted from positive wells and further diluted to isolate in a repetition of the same assay with producer and indicator cells the specific recombinant with differentiation promoting activity. RANKL (Lacey, D. L. et al., Cell 93:165-76 (1998)) is itself an example of a positive regulator of osteoclast differentiation that could have been isolated through this method.

Secreted molecules that inhibit differentiation. RAW cells or other progenitor cells transfected with TRAP/luciferase or similar indicator construct are treated with an agent that induces differentiation, in a preferred embodiment with RANKL at the lowest concentration that, in the absence of vaccinia recombinants, reproducibly induces differentiation and a positive indicator signal in every microculture of producer and indicator cells. Under these conditions, only microcultures that include a producer cell infected with a regulator polypeptide that leads to membrane expression or secretion of an inhibitor of osteoclast differentiation to the mature TRAP expressing phenotype will fail to induce luciferase synthesis and, upon addition of luciferase assay reagents, will not give rise to a readily detectable signal. Recombinant vaccinia viruses are extracted from these negative wells and further diluted to isolate in a repetition of the same assay with producer and indicator cells the specific recombinant with differentiation inhibiting activity. Osteoprotegerin (OPG), Simonet, W. S. et al., Cell 89:309-19 (1997), which is identical to osteoclastogenesis inhibitory factor (OCIF), Yasuda, H. et al., Endocrinology 139:1329-37 (1998), is an example of a type of negative regulator of osteoclast differentiation that can be isolated through this method.

### 7.4 Vector Construction

TRAP/DT-A. The pTH-1 vector has been described (Maxwell, I. H. et al., Cancer Research 46:4660-4664(1986)). This vector contains the diptheria toxin A chain gene, with expression controlled by the human metallothionein IIA promoter. The metallothionein IIA promoter is excised from this vector by digestion with XmaIII and NcoI, and replaced with another promoter. The pTH-1 vector is digested with XmaIII, blunt ended with T4 DNA Polymerase, and then digested with NcoI. These manipulations remove the metallothionein IIA promoter, and leave the vector with a 5′ blunt end, and a 3′ NcoI overhang. The TRAP(-1846-+2) promoter is excised from pBSmTRAP5′ ( Reddy, S. V. et al., J. Bone and Mineral Research 8:1263-1270 (1993)) with SmaI and BglII. The TRAP promoter is prepared for insertion into pTH-1 by ligation of an oligodeoxynucleotide adapter that converts the BglII overhang into a NcoI overhang. This adapter is constructed from 2 single stranded oligodeoxynucleotides. BglII-NcoI Sense:5′ GATCTCGGTAACCGC 3′(SEQ ID NO:105); BglII-NcoI Antisense:5′ CATGGCGGTTACCGA 3′ (SEQ ID NO:106). These two oligos are annealed together, and then ligated onto the TRAP molecule using T4 DNA Ligase. The modified TRAP is then inserted into the blunt/NcoI sites of pTH- 1.

Other DT-A constructs, pIBI30-DT-A, and a plasmid with an attenuated DT-A sequence, pIBI30-176 have been reported (Palmiter et al., Cell 50:43 5-43 (1987)). One possible advantage of the attenuated sequence is that a transfectant with leaky expression is less likely to undergo spontaneous lysis.

TRAP/Luciferase. The pKB5 vector was constructed by insertion of the mouse TRAP promoter (−1846 bp to +2 bp (positions are relative to the ATG start codon of TRAP)) into the KpnI and BglII sites of the pGL2 Basic vector (Promega).In this vector the TRAP promoter controls expression of the luciferase gene. Construction of this vector has been described (Reddy, S. V. et al., J. Bone and Mineral Research 8:1263-1270 (1993)).

GST-OPGL. For synthesis of murine and human RANKL in bacteria, the murine and human OPGL cDNA was cloned into the SmaI and Hind3 sites of pGEX-2TK (Amersham Pharmacia) to generate a GST fusion protein. Following purification of the fusion protein on glutathione sepharose, the glutathione S-tyransferase (GST) affinity tag is separated from the recombiannt protein by digestion with thrombin. Approximately 30 mg of purified RANKL can be recovered from a 1 liter bacterial culture.

### 7.5 Mesenchymal Stem Cells and Their Role in the Musculoskeletal System

Mesenchymal stem cells are pluripotent and have the capacity to differentiate into mature cells with the phenotypic expression of fat, muscle, bone, cartilage, ligament, and tendon (Gerson, S. et al., Nature Med. 5:262-64 (1999); Majumdar, M. et al., J. Cell. Physiol. 176:57-66 (1998)). Mesenchymal stem cells are critical during limb development and populate the limb bud, giving rise to the various mature mesenchymal tissues in the limb (Johnson, R., and Tabin, C., Cell 90:979-990 (1997)). The signals necessary for this process are poorly defined but are recapitulated in adult tissues during skeletal repair processes.

Mesenchymal stem cells remain in post-embryonic tissues and are present in periosteum, perichondrium, muscle, bone marrow and at other sites (Bruder, S. et al., J. Cell. Biochem. 64:278-94 (1997)). These cells retain the capacity to undergo differentiation and develop the characteristics of differentiated cells necessary for skeletal repair processes. Successful skeletal repair involves the capacity of these cells to respond to appropriate stimuli. Fracture healing is an example of this process, whereby mesenchymal cells proliferate, undergo chondrogenesis, with subsequent bone formation occurring by endochondral ossification. Ultimately this results in fracture union and healing with subsequent remodeling of the new bone. More complete knowledge of the genes involved in this process will provide targets to improve repair processes and provide the possibility of therapeutic intervention.

In other diseases of the musculoskeletal system, adequate repair rarely, if ever, occurs. An example of inadequate repair involves repair of articular cartilage defects. Joint formation is completed during embryologic development and the joint surface is composed of articular chondrocytes embedded in a highly specialized matrix. Articular cartilage is a low friction surface that is highly resistant to compressive and shear forces. Mature articular chondrocytes are terminally differentiated and have little capacity to initiate repair. Loss of the articular surface, with exposure of the underlying subchondral bone, occurs with increasing frequency with aging and is the pathological process that occurs in osteoarthritis.

Currently there are several therapies that have been used to repair articular cartilage defects, but none of these treatments have had a high degree of efficacy. In a procedure call mosaic-plasty, cores of articular cartilage and underlying bone are taken from one location and transplanted to a new location, filling in an articular cartilage defect. Frequently, several separate cores are required to fill a defect. While there is an attempt to harvest the tissue from sites with minimal need for the cartilage, this procedure has significant donor morbidity. Similarly, while there is an attempt to match the donor cartilage to the normal contour of the cartilage defect, incongruency of the repaired cartilage inevitably remains and the wear resistance of the transplanted tissue is limited.

Other procedures currently in use depend upon the development of normal tissue from transplanted cells. In the first case, terminally differentiated articular chondrocytes are harvested from a joint surface, the cell population expanded in culture, and transplanted into the defective surface (Brittberg, M. et al., N. Eng. J. Med. 331:889-895 (1994)). The cells are placed under a covering of periosteum. Although early results suggested excellent reconstitution of the tissue, later results are less promising (Buckwalter, J. Bull. Am. Acad. Orthop. Surg. 44:24-26 (1996)). In the second case, periosteum is harvested from the bone surface and placed over the cartilage defect with the cambium layer, which contains the highest proportion of mesenchymal cells, facing the defect. In both of these cases, the cellular transplants are performed in association with preparation of the underlying subchondral bone surface. However, instead of forming a hyaline cartilage surface with a high content of aggregating proteoglycans, a fibrocartilaginous reparative tissue, characterized by the expression of type I collagen and an absence of aggregating proteoglycans, forms. This tissue has inferior mechanical properties compared to normal articular cartilage. Similar results have been reported in combination with cell and perichondrial tissue transplantation. Since one of the important differences between fibrocartilage and hyaline cartilage is the production of type II collagen and aggrecan by hyaline cartilage, identification of genes and signals important in the maintenance of these genes could have tremendous clinical relevance for the development of effective reparative tissue.

Chondrogenesis. Chondrogenesis is the formation of cartilage cells and tissues from mesenchymal stem cells. At an early stage of limb development mesenchymal cells condense and shift from the production of type I to type II collagen (Erlebacher, A. et al, Cell 80:371-378 (1995)). The cells also begin to produce and secrete aggregating proteoglycans. A highly cellular and distinct lining tissue surrounds this early cartilage anlagen, which is the earliest precursor to the skeleton. This lining tissue persists and becomes the periosteum, in areas where it surrounds bone, and the perichondriun, in areas where it surrounds cartilage. The periosteal and perichondrial tissue contains mesenchymal stem cells and during development, additional cartilage cells differentiate form this tissue as the skeleton increases in width during development (Erlebacher, A. et al., Cell 80:371-378 (1995)). In the adult, this tissue provides a reservoir of cells for skeletal repair processes.

As development proceeds, the chondrocytes undergo a process of maturation that results in endochondral bone formation. In the center of the cartilaginous anlagen, chondrocytes hypertrophy, and increase approximately 5 to 10-fold in size. Associated with cell hypertrophy is an increase in alkaline phosphatase activity and the expression of type X collagen. Type X collagen is a globular collagen which is expressed only in chondrocytes undergoing terminal differentiation and committed to completion of endochondral ossification (Castagnola, P. et al., J. Cell Biol 102:2310-2317 (1986)). Although the mechanisms involved in the process are not understood, the phenotypic changes are essential for normal bone development and defects in type X collagen expression are associated with chondrodysplasias (Warman, M. L. et al., Nature Genet. 5:79-82 (1993)). Terminally differentiated chondrocytes undergo apoptosis and the calcified cartilage serves as a template for the primary bone formation. Vascular ingrowth into the region of calcified cartilage precedes bone formation. As the central region of the bone becomes ossified, the cartilaginous regions move toward opposite ends of the long bone and constitute the growth plate which is necessary for skeletal growth throughout development. The process of chondrocyte hypertrophy and terminal differentiation continues through adolescence. The entire process is recapitulated during fracture healing.

### 7.6 C3H10T½ Cells: A Model for Chondrogenesis and Osteoblastogenesis.

Several cell lines have been used to study chondrogenesis and the factors associated with this process. C3H10T½ cells are a multipotential murine embryonic mesenchymal cell line with the potential to undergo chondrogenesis, osteogenesis, myogenesis, and adipogenesis (Denker, A. et al., Differentiation 64:67-76 (1999)). These cells can undergo muscle differentiation and myotubule formation following treatment with 5-azacytidine. Chondrogenesis and adipocitogenesis also occur following this treatment (Taylor, S. and Jones, P., Cell 17:771-79 (1979)). C3H10T½ cells are particularly responsive to differentiation following treatment with bone morphogenetic proteins (BMPs). In the presence of BMPs the cells can undergo differentiation along three lineages (Atkinson, B. et al. J. Cell Biochem. 65:325-39 (1997); Katagiri, T. et al., Biochem. Biophys. Res. Commun. 172:295-299 (1990); Wang, E. et al., Growth Factors 9:57-71 (1993)), although myogenic differentiation is inhibited. However, in high density cultures, BMP treatment preferentially favors chondrogenesis. TGF-β also stimulates chondrogenesis in these cells, as does azacytadine. Similar to primary mesenchymal cells, N-cadherin is induced during chondrogenesis and appears to play an important role in this process (Haas, A. and Tuan, R. Differentiation 64:77-89 (1999)).

Sox 9 is a member of the Sox family, a group of transcription factors important in developmental processes (Pevny, L. and Lovell-Badge, R. Curr. Opin. Genet. Dev. 7:338-44 (1997)). Sox9 expression is high in chondroprogenitor cells and in chondrocytes during endochondral bone formation (Wright, E. et al., Nat. Genet. 9:15-20 (1995)). Sox9 appears to be an important regulator of type II collagen, a chondrocyte specific gene (Lefebvre, V. et al., Mol. Cell Biol. 17:2336-2346 (1997)). Zehentner, B. et al., J. Bone Min. Res. 14:1734-1741 (1999) have recently shown that BMP-2 causes a4-fold induction in Sox9 expression in C3H10T½ cells and a marked up-regulation of type II collagen gene expression. While the plating density of the C3H10T½ cells was not defined in this study, low levels of type II collagen were expressed under basal conditions. Surprisingly, type X collagen, a marker of a differentiated chondrocyte committed to endochondral bone formation, was induced. In control cultures, no type X collagen could be observed, while high levels were observed following BMP-2 treatment (200 ng/ml). Anti-sense oligonucleotides to Sox9 partially inhibited the induction of type II and type X collagen expression (Zehentner, B. et al, J. Bone Min. Res. 14:1734-1741 (1999)). Thus, marked induction of chondrocyte specific genes occurs in C3H10T½ cells following BMP-2 treatment. The hedgehog proteins can synergistically enhance differentiation of C3H10T/½ cells (Nakamura, T. et al., Biochem. Biophys. Res. Commun. 247:465-69 (1997)).

Osteoblast differentiation has been characterized in C3H10T½ cells (Katagiri, T. et al., Biochem. Biophys. Res. Commun. 172:295-299 (1990); Wang, E. et al., Growth Factors 9:57-71 (1993); Harada, H. et al., J. Biol. Chem. 274:6972-6978 (1999)). BMP-2 stimulates the differentiation of osteoblasts, and differential display has been used with C3H10T½ cells to clone osteoblast-specific genes following differentiation (Kobayashi, T. et al., Gene 198:341-49 (1997)). The osteoblast phenotype is characterized by the expression of several genes, including alkaline phosphatase, osteocalcin, and osteopontin. CBFA1 (core-binding factor) has been identified as a transcription factor essential for osteoblast differentiation. Targeted disruption of this gene in mice results in the absence of osteoblast formation (Komori, T. et al., Cell 89:755-64 (1997)) and this gene is involved in the human disorder cleidocranial dysplasia (Lee, B. et al., Nat. Genet 16:307-10 (1997)). Recently, it has been shown that co-transfection of BMP-4 and CBFA1 synergistically enhanced the expression of the osteocalcin, osteopontin, alkaline phosphatase, and type I collagen genes. The expression of osteocalcin, alkaline phosphatase, and osteopontin were undetectable in mock-transfected cells, but were highly expressed in the CBFA1 and BMP-4 transfected cells (Harada, H. et al., J. Biol. Chem. 274:6972-6978 (1999)).

Osteoarthritis and type X collagen expression. Chondrocytes express type II collagen, and are distinguished from other mesenchymal cells by the expression of this structural collagen. Chondrocytes can further differentiate into cells that calcify cartilage, ultimately leading to bone formation. This process is called endochondral ossification. Chondrocytes which undergo endochondral ossification, such as growth plate chondrocytes or chondrocytes in skeletal repair processes (fracture healing) express type X collagen. Articular chondrocytes (which line the joint) do not express type X collagen, but in arthritis, these cells begin to express this gene. Thus, type X collagen is a marker of both reparative and disease processes involving chondrocytes.

C3H10T½ cells are a multipotential murine embryonic mesenchymal cell line that normally express type I collagen and are induced to express type II collagen when they undergo chondrogenesis. Chondrogenesis is enhanced by high density plating of the cultures and by growth factors. Zehentner et al., J. Bone Min. Res. 14:1734-1741, (1999) show that BMP-2 markedly enhances the expression of type II collagen. Even more importantly, type X collagen, which cannot be detected in control cultures, is strongly expressed in the treated cultures. Other markers of chondrogenic differentiation, including aggrecan, are markedly induced.

Detection and isolation of genes that positively or negatively regulate differentiation of chondrocytes and osteoblasts. As described earlier, the invention comprises methods to detect and isolate regulator molecules, e.g., regulator polypeptides or regulator U1 SnRNAs that either positively or negatively regulate stem cell differentiation including genes that are expressed in the differentiating cell itself and that are a secreted or membrane product of another producing cell that influences differentiation in a paracrine fashion. In a preferred embodiment, the method is applied to detect and isolate recombinant vaccinia viruses expressing regulator molecules that regulate differentiation of chondrocytes and osteoblasts. One or more cell types or cell lines are required that can be induced to differentiate into chondrocytes or osteoblasts in response to a specific signal. In a preferred embodiment, high density cultures of C3H10T½ cells are induced by BMP-2 to differentiate into chondrocytes. In another preferred embodiment continued differentiation of the same pluripotent C3H10T½ cells into osteoblasts is induced by TGFβ (Joyce, M. et al., J. Cell Biol. 110:2195-207 (1990)). Further discrimination in the readout of cell differentiation is possible by employing C3H10T½ cells transfected with promoter/suicide or promoter/indicator constructs (as previously described for isolation of genes that regulate osteoclast differentiation) where, in this case, the promoter is specific for expression of either a marker of chondrocyte differentiation or a marker of osteoblast differentiation. As markers of chondrocyte differentiation, type II collagen or aggrecan are preferred, and type X collagen is especially preferred. As a marker of osteoblast differentiation, osteocalcin is especially preferred.

The most important and meaningful information regarding the collagen promoter construct is whether or not it is expressed in a manner consistent with the in vivo expression pattern. If it is not, then it is uncertain that it would be a good marker or endpoint for the differentiated phenotype. Tissue specific expression patterns have been examined in mice transgenic for either the mouse type X collagen promoter (Eerola, I. et al., Ann. NY Acad Sci. 785:248-50(1996)) or the chicken type X promoter (Jacenko, O et al., Nature 365:56-61, (1993)). Interestingly, the chicken type X collagen promoter (in the mouse), provides an expression pattern identical to the in vivo expression of the mouse type X collagen gene. The mouse type X collagen promoters tested were expressed in a number of different tissues, including brain, skin, and in some cases hypertrophic chondrocytes. More importantly, a mutation that should cause a chondrodysplasia (and does in the chicken constructs) did not cause this using the mouse sequences. Thus, the chicken promoter, at least, appears to offer expression with the specificity of the normal gene. The mouse promoter appears to be less specific. The chicken type X collagen promoter is preferred for this embodiment of the invention.

### 7.7 Vector Construction

Osteocalcin-DT-A. The OC2 promoter is excised from pOC2CAT with XhoI and HindIII. Adapters is ligated onto this molecule in order to convert the XhoI overhang into a XmaIII overhang. This is done using oligos XhoI-XmaIII sense: 5′ GGCCGAAATAACCGC 3′ (SEQ ID NO:107), and XhoI-XmaIII antisense:5′ TCGAGCGGTTATTTC 3′ (SEQ ID NO:108). The HindIII overhang is converted into a NcoI overhang using oligos H3-NcoI sense 5′ AGCTTCGGTAACCGC 3′ (SEQ ID NO:109), and H3-NcoI antisense 5′ CATGGCGGTTACCGA 3′ (SEQ ID NO:110). These adapters are annealed together, and then ligated onto the OC2 molecule. The adapter modified OC2 promoter is then inserted into the XmaIII and NcoI sites of pTH-1.

Osteocalcin-Luciferase. The pGL3-Basic Vector (Promega) contains a promoterless luciferase gene. The 1.Kb Osteocalcin promoter has been described (Frenkel, B. et al., Endocrinology 138:2109-2116 (1997)). The OC2 promoter is available in vector pOC2-CAT. The OC2 promoter is excised from this vector with XhoI and HindIII, and inserted into the matching XhoI and HindIII sites of pGL3-Basic Vector. This new vector, pOC2-Luc, has the luciferase gene controlled by the OC2 promoter.

Chicken CollagenX-DT-A. The B640-CAT construct has been described (Volk, S. W. et al., J. Bone Min. Res. 13:1521-1529 (1998)). This vector contains the Chick Collagen X “B” Fragment/promoter controlling expression of the CAT gene. The “B” Fragment/promoter is excised from this construct using PstI and SalI. Adapters are ligated onto this molecule in order to convert the PstI overhang into a XmaIII overhang. This is done using oligos PstI-XmaIII sense: 5′ GGCCGGAAATAACCGCTGCA 3′ (SEQ ID NO:111), and PstI-XmaIII antisense:5′ GCGGTTATTTCC 3′ (SEQ ID NO:112). The SalI overhang will be converted into a NcoI overhang using oligos SalI-NcoI sense 5′ CTGAGGAAATAACCGC 3′ (SEQ ID NO:113), and SalI-NcoI antisense 5′ CATGGCGGTTATTTCC 3′ (SEQ ID NO:114). These adapters are annealed together, and then ligated onto the Chick Collagen X promoter molecule. The adapter modified Chick Collagen X promoter is then inserted into the XmaIII and NcoI sites of pTH-1.

Chicken Collagen-X-Luciferase. The B640-Luciferase was constructed by insertion of the 1610 bp upstream “B” fragment and promoter of Chick Collagen X into the SpeI and SalI sites of pRLnull (Promega). In this vector the Chick Collagen X “B” Fragment/promoter controls expression of the luciferase gene. Construction of this vector has been described (Volk, S. W. et al., J. Bone Min. Res. 13:1521-1529 (1998)).

All of the elements required to apply the methods of this invention to detect and isolate genes that regulate differentiation of chondrocytes and osteoblasts are available: (i) Precursor cells, C3H10T½, can be induced to differentiate into either chondrocytes or osteoblasts by addition of well-defined soluble factors, BMP-2 under high density culture conditions for chondrocytes and TGFβ for osteoblasts; (ii) tissue-specific markers of differentiation are known, type X collagen for chondrocytes and osteocalcin for osteoblasts, whose promoters have been isolated and can be employed for construction of differentiation sensitive suicide or other reporter gene constructs; (iii) representative vaccinia cDNA libraries of regulator molecules, e.g., regulator polypeptides or regulator U1 SnRNAs are constructed as described herein. Employing these reagents, all of the same strategies previously described to detect and isolate regulator molecules that regulate osteoclast differentiation can be applied to chondrocyte and osteoblast differentiation. Some issues of special interest in this situation include whether differentiated osteoblasts express factors that inhibit differentiation to chondrocytes and vice versa. Examples of positive regulators of differentiation that could have been isolated through this method include CBFA1 (Mundlos, S. et al., Cell 89:773 (1997); Otto, F. et al., Cell 89:765 (1997); Inada, M. et al., Dev Dyn 214:279 (1999)); Ihh, indian hedgehog signaling (Vortkamp, A. et al., Science 273:613 (1996); St-Jacques, B. et al., Genes Dev 13:2072 (1999)); and PTHrP, parathyroid hornone-related peptide (Lanske, B. et al., J Clin Invest 104:399 (1999); Karaplis, A. C. et al., Genes Dev 8:277 (1994)).

Human differentiation factors and stem cells. The C3H10T½ precursor to osteoblasts and chondrocytes and the previously described RAW precursor to osteoclasts are of murine origin. Although the gene products identified through use of these cell lines will also be of murine origin, there are strong and numerous precedents for homology between factors that regulate differentiation of homologous tisues in mice and humans. In general, the murine genes isolated can be used to isolate human homologs which can then be tested for the ability to regulate differentiation of the corresponding human stem cells. In an increasing number of instances human stem cells are becoming available. In particular several human stem cell lines have been recently isolated by SV40 transformation from both embryonic cartilage and adult cartilage, Moulton, P. J. et al., British Journal of Rheumatology 36(5):522-529 (1997); Goldring, M. B. and Berenbaum, F. Osteoarthritis & Cartilage 7(4):386-388 (1999)). These cell lines will have to be induced to express type X collagen. It is expected that they will provide suitable human material to directly detect and isolate human genes that regulate chondrocyte and osteoblast differentiation.

### Example 8

### Host Cells

Cells and cells lines for use as host or recipient or library cells according to the present invention include those disclosed in scientific literature such as American Type Culture Collection publications including American Type Culture Collection Catalogue of Cell Lines and Hybridomas, 7th Ed., ATCC, Rockville, Md. (1992), which literature lists deposited and commercially available cell lines as well as culture conditions, and additional references.

For example, host cells according to the present invention include the monkey kidney cell line, designated “COS,” including COS cell clone M6. COS cells are those that have been transformed by SV40 DNA containing a functional early gene region but a defective origin of viral DNA replication. Also preferred are murine “WOP” cells, which are NIH 3T3 cells transfected with polyoma origin deletion DNA.

Other examples of host cells for use in the disclosed methods are monkey kidney CVI line transformed by SV40 (COS-7, ATCC CRL 165 1); human embryonic kidney line (293, Graham, et al., J. Gen Virol 36:59 (1977)); baby hamster kidney cells (BHK, ATCC CCL 10); chinese hamster ovary-cells-DHFR (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci. 383:44-68 (1982)); human B cells (Daudi,ATCC CCL 213); human T cells (MOLT-4, ATCC CRL 1582); and human macrophage cells (U-937, ATCC CRL 1593).

Preferred cell types for use in the invention will vary with the cellular phenotype to be modified. Suitable cells include, but are not limited to, mammalian cells, including animal (rodents, including mice, rats, hamsters and gerbils), primates, and human cells, particularly including tumor cells of all types, including breast, skin, lung, cervix, colorectal, leukemia, brain, etc.

The murine stem cell line RAW (Hsu, H. et al., Proc Natl Acad Sci USA 96(7):3540-45 (1999); Owens, J. M. et al., J Cell Physiol 179:170 (1999)) and pluripotent stem cell line C3H10T½ (Denker, A. et al., Differentiation 64:67-76 (1999)) are especially preferred for studies of osteoclast and chondrocyte or osteoblast differentiation.

However, the choice of cells or cell lines is not limited to those described herein, and may be any cell or cell line. As indicated below, the choice depends on the system under study, or the particular polynucleotide which is desired to be isolated.

As another example, to isolate a polynucleotide which is growth suppressive or toxic in breast cancer, it is preferable to use as host cells breast cancer cell lines such as 21NT, 21PT, 21MT-1, AND 21MT-2. Band et al., Cancer Res. 50:7351-7 (1990). Once a growth suppressive polynucleotide is isolated, it may be tested in non transformed controls, such as normal breast epithelial cell line H16N2, to determine whether its growth suppressive activity is specific for tumor cells.

Many cell types can be used in the selection method of the invention. Cells include dividing cells, non dividing cells, terminally differentiated cells, pluripotent stem cells, committed progenitor cells and uncommitted stem cells.

Cells and cell types also include muscle cells such as cardiac muscle cells, skeletal muscle cells and smooth muscle cells; epithelial cells such as squamous epithelial cells, including endothelial cells, cuboid epithelial cells and columnar epithelial cells; nervous tissue cells such as neurons and neuroglia.

Cells that can be used in the selection method of the present invention also include nervous system cells such as neurons, including cortical neurons, inter neurons, central effector neurons, peripheral effector neurons and bipolar neurons; and neuroglia, including Schwann cells, oligodendrocytes, astrocytes, microglia and ependyma.

Additionally, endocrine and endocrine-associated cells may also be used such cells as pituitary gland cells including epithelial cells, pituicytes, neuroglia, agranular chromophobes, granular chromophils (acidophils and basophils); adrenal gland cells including epinephrine-secreting cells, non-epinephrine-secreting cells, medullary cells, cortical cells (cells of the glomerulosa, fasciculata and reticularis); thyroid gland cells including epithelial cells (principal and parafollicular); parathyroid gland cells including epithelial cells (chief cells and oxyphils); pancreas cells including cells of the islets of Langerhans (alpha, beta and delta cells); pineal gland cells including parenchymal cells and neuroglial cells; thymus cells including parafollulicular cells; cells of the testes including seminiferous tubule cells, interstitial cells (“Leydig cells”), spermatogonia, spermatocytes (primary and secondary), spermatids, spermatozoa, Sertoli cells and myoid cells; cells of the ovary including ova, oogonia, oocytes, granulosa cells, theca cells (internal and external), germinal epithelial cells and follicle cells (primordial, vesicular, mature and atretic).

Also included are muscle cells such as myofibrils, intrafusal fibers and extrafusal fibers; skeletal system cells such as osteoblasts, osteocytes, osteoclasts and their progenitor cells.

Circulatory system cells are also included such cells as heart cells (myocardial cells); cells of the blood and lymph including erythropoietin-sensitive stem cells, erythrocytes, leukocytes (such as eosinophils, basophils and neutrophils (granular cells) and lymphocytes and monocytes (agranular cells)), thrombocytes, tissue macrophages (histiocytes), organ-specific phagocytes (such as Kupffer cells, alveolar macrophages and microglia), B-lymphocytes, T-lymphocytes (such as cytotoxic T cells, helper T cells and suppressor T cells), megaloblasts, monoblasts, myeloblasts, lymphoblasts, proerythroblasts, megakaryoblasts, promonocytes, promyelocytes, prolymphocytes, early normoblasts, megakaryocytes, intermediate normoblasts, metamyelocytes (such as juvenile metamyelocytes, segmented metamyelocytes and polymorphonuclear granulocytes), late normoblasts, reticulocytes and bone marrow cells.

Respiratory system cells are also included such as capillary endothelial cells and alveolar cells; as are urinary system cells such as nephrons, capillary endothelial cells, granular cells, tubule endothelial cells and podocytes; digestive system such as simple columnar epithelial cells, mucosal cells, acinar cells, parietal cells, chief cells, zymogen cells, peptic cells, enterochromaffin cells, goblet cells, Argentaffen cells and G cells; and sensory cells such as auditory system cells (hair cells); olfactory system cells such as olfactory receptor cells and columnar epithelial cells; equilibrium/vestibular apparatus cells including hair cells and supporting cells; visual system cells including pigment cells, epithelial cells, photoreceptor neurons (rods and cones), ganglion cells, amacrine cells, bipolar cells and horizontal cells are also included.

Additionally, mesenchymal cells, stromal cells, hair cells/follicles, adipose (fat) cells, cells of simple epithelial tissues (squamous epithelium, cuboidal epithelium, columnar epithelium, ciliated columnar epithelium and pseudostratified ciliated columnar epithelium), cells of stratified epithelial tissues (stratified squamous epithelium (keratinized and non-keratinized), stratified cuboidal epithelium and transitional epithelium), goblet cells, endothelial cells of the mesentery, endothelial cells of the small intestine, endothelial cells of the large intestine, endothelial cells of the vasculature capillaries, endothelial cells of the microvasculature, endothelial cells of the arteries, endothelial cells of the arterioles, endothelial cells of the veins, endothelial cells of the venules, etc.;cells of the connective tissue include chondrocytes, adipose cells, periosteal cells, endosteal cells, odontoblasts, osteoblasts, osteoclasts and osteocytes; endothelial cells, hepatocytes, keratinocytes and basal keratinocytes, muscle cells, cells of the central and peripheral nervous systems, prostate cells, and lung cells, cells in the lung, breast, pancreas, stomach, small intestine, and large intestine; epithelial cells such as sebocytes, hair follicles, hepatocytes, type II pneumocytes, mucin-producing goblet cells, and other epithelial cells and their progenitors of the skin, lung, liver, and gastrointestinal tract may be used in the methods of the present invention, preferably the selection and screening methods.

The cells may be in any cell phase, either synchronous or not, including M, G1, S, and G2. In a preferred embodiment, cells that are replicating or proliferating are used. Alternatively, non-replicating cells may be used.

### Example 9

### Expression Profiling

Many of the identification (e.g., screening and/or selection) methods described herein depend on expression of host cell genes or target host cell transcriptional regulatory regions, which are directly or indirectly influenced by regulator molecules. It is important to note that most preferred embodiments of the present invention require that host cells be infected with a eukaryotic virus vector, preferably a poxvirus vector, and even more preferably a vaccinia virus vector. It is well understood by those of ordinary skill in the art that some host cell protein synthesis is rapidly shut down upon poxvirus infection in some cell lines, even in the absence of viral gene expression. This problem is not intractable, however, because in certain cell lines, inhibition of host protein synthesis remains incomplete until after viral DNA replication. See Moss, B., “Poxviridae and their Replication” in Virology, 2d Edition, Fields, B. N. and Knipe, D. M. et al., Eds., Raven Press, p. 2096 (1990). There is a need, however, to rapidly screen a variety of host cells for their ability to express gene products which are upregulated by a regulator molecule upon infection by a eukaryotic virus vector, preferably a poxvirus vector, and even more preferably a vaccinia virus vector; and to screen desired host cells for differential expression of cellular genes upon virus infection with various mutant and attenuated viruses.

Accordingly, a method is provided for screening a variety of host cells for the expression host cell genes and/or the operability of target host cell transcriptional regulatory regions effecting a particular phenotype, upon infection by a virus vector, through expression profiling of particular host cells in microarrays of ordered cDNA libraries. Expression profiling in microarrays is described in Duggan, D. J. et al., Nature Genet. 21(1 Suppl):10-14 (1999), which is incorporated herein by reference in its entirety.

According to this method, expression profiling is used to compare host cell gene expression patterns in uninfected host cells and host cells infected with a eukaryotic virus expression vector, preferably a poxvirus vector, even more preferably a vaccinia virus vector, where the particular eukaryotic virus vector is the vector used to construct the regulator molecule library of the present invention. In this way, suitable host cells which continue to undergo expression of the necessary inducible proteins upon infection with a given virus, can be identified.

Expression profiling is also used to compare host cell gene expression patterns in a given host cell, for example, comparing expression patterns when the host cell is infected with a fully infectious virus vector, and when the host cell is infected with a corresponding attenuated virus vector. Expression profiling in microarrays allows large-scale screening of host cells infected with a variety of attenuated viruses, where the attenuation is achieved in a variety of different ways, as described above. For example, certain attenuations are achieved through genetic mutation. Many vaccinia virus mutants have been characterized. These may be fully defective mutants, i.e., the production of infectious virus particles requires helper virus, or they may be conditional mutants, e.g., temperature sensitive mutants. Conditional mutants are particularly preferred, in that the virus-infected host cells can be maintained in a non-permissive environment, e.g., at a non-permissive temperature, during the period where host gene expression is required, and then shifted to a permissive environment, e.g., a permissive temperature, to allow virus particles to be produced. Alternatively, a fully infectious virus may be “attenuated” by chemical inhibitors which reversibly block virus replication at defined points in the infection cycle. Chemical inhibitors include, but are not limited to hydroxyurea and 5-fluorodeoxyuridine. Virus-infected host cells are maintained in the chemical inhibitor during the period where host gene expression is required, and then the chemical inhibitor is removed to allow virus particles to be produced.

Using this method, expression profiling in microarrays may be used to identify suitable host cells, suitable transcription regulatory regions, and/or suitable attenuated viruses in any of the selection/screening methods described herein.

### Example 10

### Attenuation of Poxvirus Mediated Host Shut-off by Reversible Inhibitor of DNA Synthesis

As discussed infra, attenuated or defective virus is sometimes desired to reduce cytopathic effects. Cytopathic effects during poxvirus infection might interfere with selection and identification of regulator molecules that regulate specific gene expression in the host cell. Such effects can be attenuated with a reversible inhibitor of DNA synthesis such as hydroxyurea (HU) (Pogo, B. G. and Dales, S. Virology, 43(1):144-51 (1971)). HU inhibits both cell and viral DNA synthesis by depriving replication complexes of deoxyribonucleotide precursors (Hendricks, S. P. and Mathews, C. K. J. Biol. Chem, 273(45):29519-23 (1998)). Inhibition of viral DNA replication blocks late viral RNA transcription while allowing transcription and translation of genes under the control of early vaccinia promoters (Nagaya et al., Virology 40(4):1039-51 (1970)). Thus, treatment with reversible inhibitor of DNA synthesis such as HU allows the detection of effects of regulator molecules (under the control, for example, of an early viral promoter) on host gene expression. Following appropriate incubation, HU inhibition can be reversed by washing the host cells so that the viral replication cycle continues and infectious recombinants can be recovered (Pogo, B. G. and Dales, S. Virology 43(1):144-51 (1971)).

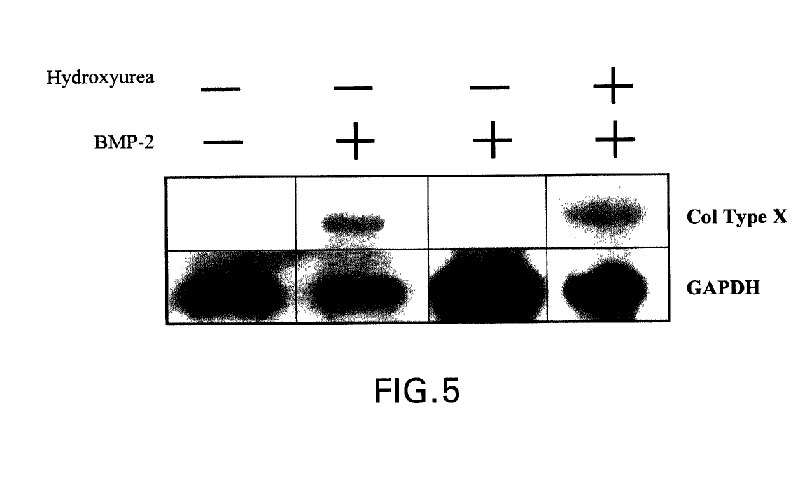

The results in FIG. 5 demonstrate that induction of type X collagen synthesis, a marker of chondrocyte differentiation, in C3H10T1/2 progenitor cells treated with BMP-2 (Bone Morphogenetic Protein-2) is blocked by vaccinia infection but that its synthesis can be rescued by HU mediated inhibition of viral DNA synthesis. When HU is removed from cultures by washing with fresh medium, viral DNA synthesis and assembly of infectious particles proceeds rapidly so that infectious viral particles can be isolated as soon as 2 hrs post-wash.

C3H10T½ cells were infected with WR vaccinia virus at MOI=1 and 1 hour later either medium or 400 ng/ml of BMP-2 in the presence or absence of 2 mM HU was added. After a further 21 hour incubation at 37° C., HU was removed by washing with fresh medium. The infectious cycle was allowed to continue for another 2 hours to allow for initiation of viral DNA replication and assembly of infectious particles. At 24 hours RNA was extracted from cells maintained under the 4 different culture conditions. Northern analysis was carried out using a type X collagen specific probe. The uninduced C3H10T½ cells have a mesenchymal progenitor cell phenotype and as such do not express type X collagen (first lane from left). Addition of BMP-2 to normal, uninfected C3H10T½ cells induces differentiation into mature chondrocytes and expression of type X collagen (compare first and second lanes from left), whereas addition of BMP-2 to vaccinia infected C3H10T½ cells fails to induce synthesis of type X collagen (third lane from left). In the presence of 2mM HU, BMP-2 induces type X collagen synthesis even in vaccinia virus infected C3H10T½ cells (fourth lane from left).

This strategy for attenuating viral cytopathic effects is applicable to other cell types and to selection of regulator molecules that regulate expression of other host genes.

The present invention is not to be limited in scope by the specific embodiments described which are intended as single illustrations of individual aspects of the invention, and any constructs, viruses or enzymes which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference. The disclosure and claims of U.S. application Ser. No. 08/935,377, filed Sep. 22, 1997, U.S. application Ser. No. 60/192,586, filed Mar. 28, 2000, U.S. application Ser. No. 60/263,226, filed Jan. 23, 2001, and U.S. application Ser. No.60/271,426, filed Feb. 27,2001 are herein incorporated by reference in their entireties.

What is claimed is:

1. A method of identifying polynucleotides which encode a regulator polypeptide, whose expression induces the activation of a target transcriptional regulatory region in a eukaryotic host cell, comprising:
(a) providing a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which results in host cell death, and wherein said selectable gene product is expressed upon activation of said target transcriptional regulatory region;
(b) introducing into said population of host cells a library of polynucleotides encoding, through operable association with a vector transcriptional regulatory region, a plurality of candidate regulator polypeptides, each candidate regulator polypeptide comprising (i) a candidate peptide, and (ii) a molecular scaffold fused to said peptide such that said peptide is displayed on the surface of said candidate regulator polypeptide;
(c) permitting expression of said plurality of candidate regulator polypeptides in said population of host cells under conditions wherein host cell death can be detected; and
(d) recovering polynucleotides of said library from those individual host cells which undergo cell death.

2. The method of claim 1, further comprising:
(e) providing a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which results in host cell death, and wherein said selectable gene product is expressed upon activation of said target transcriptional regulatory region;
(f) introducing said recovered polynucleotides into said population of host cells;
(g) permitting expression of candidate regulator polypeptides encoded by said recovered polynucleotides in said population of host cells under conditions wherein host cell death can be detected; and
(h) recovering polynucleotides of said library from those individual host cells which undergo cell death.

3. The method of claim 2, further comprising repeating steps (e)-(h) one or more times, thereby enriching for polynucleotides of said library which encode regulator polypeptides whose expression induces activation of a target transcriptional regulatory region in a eukaryotic host cell.

4. The method of claim 1, further comprising isolating said recovered polynucleotides.

5. The method of claim 1, wherein expression of said selectable gene product results in cell death through a process selected from the group consisting of irreversible growth inhibition, apoptosis, and cell lysis.

6. The method of claim 1, wherein said candidate peptide comprises a random chain of at least 4 amino acids.

7. The method of claim 1, wherein said molecular scaffold comprises a conformation or motif selected from the group consisting of (a) a β-sandwich motif; (b) a zinc finger motif; and (c) an α-helical bundle motif.

8. The method of claim 1, wherein said molecular scaffold is an immunoglobulin polypeptide, or fragment thereof.

9. The method of claim 1, wherein said molecular scaffold is selected from the group consisting of a fibronectin type III domain (FN3), a minibody, tendamistat, a CP1 zinc finger, a staphylococcal protein A analog Z domain, a pancreatic secretory trypsin inhibitor (PSTI), a synthetic coiled coil, a human lipoprotein-associated coagulation inhibitor (LACI-DI), and a cytochrome b₅₆₂ protein.

10. The method of claim 9, wherein said molecular scaffold is FN3.

11. The method of claim 10, wherein said candidate peptide is fused into a region selected from the group consisting of: the FN3 BC loop, the FN3 FG loop, the FN3 terminal tail, and a combination of two or more of said regions.

12. The method of claim 1 1, wherein said combination comprises the FN3 BC loop and the FN3 FG loop.

13. The method of claim 1, wherein the candidate regulator polypeptides further comprise one or more fusion partners.

14. The method of claim 1, wherein said target cellular process is selected from the group consisting of cellular differentiation, growth regulation, apoptosis, and hormonal response.

15. The method of claim 5, wherein expression of said selectable gene product induces apoptosis.

16. The method of claim 15, wherein said selectable gene product is an apoptosis-related gene product which directly promotes apoptosis.

17. The method of claim 15, wherein said selectable gene product indirectly promotes apoptosis.

18. The method of claim 5, wherein said selectable gene product comprises a death domain containing receptor expressed on the surface of said host cells, and wherein said host cells are contacted with a ligand specific for said death domain containing receptor.

19. The method of claim 5, wherein expression of said selectable gene product results in a cytotoxic T-lymphocyte induced lytic event.

20. The method of claim 19, wherein said target transcriptional regulatory region is operably associated with a polynucleotide encoding a target epitope for a cytotoxic T lymphocyte (CTL).

21. The method of claim 20, wherein said target epitope is expressed on the surface of said host cells in the context of a native MHC molecule expressed on said host cell, and wherein said host cells are contacted with CTLs which are restricted for said MHC molecule and specific for said target epitope.

22. The method of claim 5, wherein expression of said selectable gene product results in cell suicide.

23. The method of claim 22, wherein said target transcriptional regulatory region is operably associated with a polynucleotide encoding said heterologous suicide protein.

24. The method of claim 23, wherein said heterologous suicide protein comprises a protein selected from the group consisting of a diphtheria toxin A chain polypeptide, a Pseudomonas exotoxin A chain polypeptide, a ricin A chain polypeptide, an abrin A chain polypeptide, a modeccin A chain polypeptide, and an alpha-sarcin polypeptide.

25. The method of claim 1, wherein said library of polynucleotides is introduced into said population of eukaryotic host cells by means of a eukaryotic virus vector.

26. The method of claim 25, wherein said population of eukaryotic host cells are infected with said library at a multiplicity of infection ranging from about 1 to about 10.

27. The method of claim 25, wherein said eukaryotic virus vector is an animal virus vector.

28. The method of claim 25, wherein said eukaryotic virus vector is a plant virus vector.

29. The method of claim 27, wherein said vector is capable of producing infectious virus particles in mammalian cells.

30. The method of claim 29, wherein the naturally-occurring genome of said vector is DNA.

31. The method of claim 30, wherein the naturally-occurring genome of said vector is linear, double-stranded DNA.

32. The method of claim 31, wherein said vector is selected from the group consisting of an adenovirus vector, a herpesvirus vector and a poxvirus vector.

33. The method of claim 32, wherein said vector is a poxvirus vector.

34. The method of claim 33, wherein said poxvirus vector is selected from the group consisting of an orthopoxvirus vector, an avipoxvirus vector, a capripoxvirus vector, a leporipoxvirus vector, an entomopoxvirus vector, and a suipoxvirus vector.

35. The method of claim 34, wherein said poxvirus vector is an orthopoxvirus vector selected from the group consisting of a vaccinia virus vector and a raccoon poxvirus vector.

36. The method of claim 35, wherein said poxvirus vector is a vaccinia virus vector.

37. The method of claim 36, wherein said host cells are permissive for the production of infectious virus particles of said vaccinia virus vector.

38. The method of claim 36, wherein said vaccinia virus vector is attenuated.

39. The method of claim 38, wherein said vaccinia virus vector is deficient in D4R synthesis.

40. The method of claim 33, wherein said vector transcriptional regularoty region of said library of polynucleotides functions in the cytoplasm of a poxvirus-infected cell.

41. The method of claim 40, wherein said vector transcriptional regulatory region comprises a promoter.

42. The method of claim 41, wherein said promoter is constitutive.

43. The method of claim 42, wherein said promoter is a vaccinia virus p7.5 promoter.

44. The method of claim 43, wherein said promoter is a synthetic early/late promoter.

45. The method of claim 41, wherein said promoter is a T7 phage promoter active in cells in which T7 RNA polymerase is expressed.

46. The method of claim 40, wherein said vector transcriptional regulatory region comprises a transcriptional termination region.

47. The method of claim 25, wherein said library of polynucleotides is constructed by a method comprising:
(a) Providing a population of host cells permissive for the production of infectious viral particles of said eukaryotic virus vector;
(b) cleaving an isolated linear DNA fragment comprising the genome of said eukaryotic virus vector to produce a first viral fragment and a second viral fragment, wherein said first fragment is nonhomologous with said second fragment;
(c) providing a population of transfer plasmids comprising polynucleotides encoding said plurality of candidate regulator polypeptides through operable association with a transcription control region, wherein each of said polynucleotides is flanked by a 5′ flanking region and a 3′ flanking region, wherein said 5′ flanking region is homologous to said first viral fragment and said 3′ flanking region is homologous to said second viral fragment;
(d) introducing said transfer plasmids and said first and second viral fragments into said population of host cells under conditions wherein each of said transfer plasmids, said first viral fragment, and said second viral fragment undergo in vivo homologous recombination, thereby producing a population of viable modified virus genomes, each comprising a polynucleotide which encodes a candidate regulator polypeptide; and
(e) recovering said population of modified virus genomes.

48. A kit for the identification of a regulator polypeptide, whose expression induces the activation of a target transcriptional regulatory region in a eukaryotic host cell, comprising:
(a) a library of polynucleotides encoding, through operable association with a vector transcriptional regulatory region, a plurality of candidate regulator polypeptides, each candidate regulator polypeptide comprising (i) a candidate peptide, and (ii) a molecular scaffold fused to said peptide such that said peptide is displayed on the surface of said candidate regulator polypeptide, wherein said library is constructed in a eukaryotic virus vector; and
(b) a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which results in host cell death, and wherein said selectable gene product is expressed upon activation of said target transcriptional regulatory region;
wherein polynucleotides encoding said regulator polypeptides are recoverable from individual host cells which undergo cell death.

49. An isolated polynucleotide encoding a regulator polypeptide, whose expression induces activation of a target transcriptional regulatory region in a eukaryotic host cell, produced by the method of claim 1.

50. A composition comprising the regulator polypeptide of claim 49, and a pharmaceutically acceptable carrier.

51. A method of identifying polynucleotides which encode a regulator polypeptide, whose expression influences the activation of a target transcriptional regulatory region in a eukaryotic host cell, comprising:
(a) providing a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which causes said host cells to exhibit a predetermined modified phenotype, and wherein said selectable gene product is expressed upon activation of said target transcriptional regulatory region;
(b) introducing into said population of host cells a library of polynucleotides constructed in a poxvirus vector, wherein said library of polynucleotides encode, through operable association with a poxvirus transcriptional regulatory region, a plurality of candidate regulator polypeptides, each candidate regulator polypeptide comprising (i) a candidate peptide, and (ii) a molecular scaffold fused to said peptide such that said peptide is displayed on the surface of said candidate regulator polypeptide;
(c) permitting expression of said plurality of candidate regulator polypeptides in said population of host cells under conditions wherein said modified phenotype can be detected; and
(d) recovering poxvirus vector particles comprising polynucleotides of said library from those individual host cells selected from the group consisting of: those host cells which exhibit said modified phenotype, and those host cells which fail to exhibit said modified phenotype.

52. The method of claim 51, further comprising:
(e) providing a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which causes said host cells to exhibit a predetermined modified phenotype, and wherein said selectable gene product is expressed upon activation of said target transcriptional regulatory region;
(f) introducing said recovered poxvirus vector particles into said population of host cells;
(g) permitting expression of candidate regulator polypeptides encoded by said recovered polynucleotides in said population of host cells under conditions wherein said modified phenotype can be detected; and
(h) recovering poxvirus particles comprising polynucleotides of said library from those individual host cells selected from the group consisting of: those host cells which exhibit said modified phenotype, and those host cells which fail to exhibit said modified phenotype.

53. The method of claim 52, further comprising repeating steps (e)-(h) one or more times, thereby enriching for polynucleotides of said library which encode regulator polypeptides whose expression influences activation of a target transcriptional regulatory region in a eukaryotic host cell.

54. The method of claim 51, further comprising isolating polynucleotides of said library from said recovered poxvirus vector particles.

55. The method of claim 51, wherein expression of said regulator polypeptide influences the activation of said target transcriptional regulatory region by inducing said activation, and wherein poxvirus particles are recovered from those host cells which exhibit said modified phenotype.

56. The method of claim 51 wherein expression of said regulator polypeptide influences the activation of said target transcriptional regulatory region by suppressing said activation, and wherein poxvirus particles are recovered from those host cells which fail to exhibit said modified phenotype.

57. The method of claim 51, wherein said candidate peptide comprises a random chain of at least 4 amino acids.

58. The method of claim 51, wherein said molecular scaffold comprises a conformation or motif selected from the group consisting of (a) a β-sandwich motif; (b) a zinc finger motif, and (c) an α-helical bundle motif.

59. The method of claim 51, wherein said molecular scaffold is an immunoglobulin polypeptide, or fragment thereof.

60. The method of claim 51, wherein said molecular scaffold is selected from the group consisting of a fibronectin type III domain (FN3), a minibody, tendamistat, a CP1 zinc finger, a staphylococcal protein A analog Z domain, a pancreatic secretory trypsin inhibitor (PSTI), a synthetic coiled coil, a human lipoprotein-associated coagulation inhibitor (LACI-DI), and a cytochrome b₅₆₂ protein.

61. The method of claim 60, wherein said molecular scaffold is FN3.

62. The method of claim 61, wherein said candidate peptide is fused into a region selected from the group consisting of: the FN3 BC loop, the FN3 FG loop, the FN3 terminal tail, and a combination of two or more of said regions.

63. The method of claim 62, wherein said combination comprises the FN3 BC loop and the FN3 FG loop.

64. The method of claim 51, wherein said candidate regulator polypeptides further comprise one or more fusion partners.

65. The method of claim 51, wherein said target cellular process is selected from the group consisting of cellular differentiation, growth regulation, apoptosis, and hormonal response.

66. The method of claim 51, wherein said modified phenotype is cell death.

67. The method of claim 66, wherein expression of said selectable gene product results in cell death through a process selected from the group consisting of irreversible growth inhibition, apoptosis, and cell lysis.

68. The method of claim 67, wherein expression of said selectable gene product induces apoptosis.

69. The method of claim 67, wherein said selectable gene product comprises a death domain containing receptor expressed on the surface of said host cells, and wherein said host cells are contacted with a ligand specific for said death domain containing receptor.

70. The method of claim 67, wherein expression of said selectable gene product results in a cytotoxic T-lymphocyte induced lytic event.

71. The method of claim 70, wherein said target transcriptional regulatory region is operably associated with a polynucleotide encoding a target epitope for a cytotoxic T lymphocyte (CTL).

72. The method of claim 71, wherein said target epitope is expressed on the surface of said host cells in the context of a native MHC molecule expressed on said host cell, and wherein said host cells are contacted with CTLs which are restricted for said MHC molecule and specific for said target epitope.

73. The method of claim 67, wherein expression of said selectable gene product results in cell suicide.

74. The method of claim 73, wherein said target transcriptional regulatory region is operably associated with a polynucleotide encoding said heterologous suicide protein.

75. The method of claim 74, wherein said heterologous suicide protein comprises a protein selected from the group consisting of a diphtheria toxin A chain polypeptide, a Pseudomonas exotoxin A chain polypeptide, a ricin A chain polypeptide, an abrin A chain polypeptide, a modeccin A chain polypeptide, and an alpha-sarcin polypeptide.

76. The method of claim 51, wherein said population of eukaryotic host cells is adherent to a solid support and wherein said modified phenotype is nonadherence.

77. The method of claim 51, wherein said selectable gene product comprises a membrane receptor molecule, and wherein said modified phenotype is expression of said membrane receptor molecule.

78. The method of claim 77, wherein expression of said membrane receptor molecule is detected by binding of a ligand specific for said membrane receptor molecule.

79. The method of claim 51, wherein said selectable gene product comprises a membrane receptor molecule, and wherein said modified phenotype is reduced expression of said membrane receptor molecule.

80. The method of claim 79, wherein reduced expression of said membrane receptor molecule is detected by a reduction in binding of a ligand specific for said membrane receptor molecule.

81. The method of claim 51, wherein said poxvirus vector is selected from the group consisting of an orthopoxvirus vector, an avipoxvirus vector, a capripoxvirus vector, a leporipoxvirus vector, an entomopoxvirus vector, and a suipoxvirus vector.

82. The method of claim 81, wherein said poxvirus vector is an orthopoxvirus vector selected from the group consisting of a vaccinia virus vector and a raccoon poxvirus vector.

83. The method of claim 82, wherein said poxvirus vector is a vaccinia virus vector.

84. The method of claim 83, wherein said host cells are permissive for the production of infectious virus particles of said vaccinia virus vector.

85. The method of claim 83, wherein said vaccinia virus vector is attenuated.

86. The method of claim 85, wherein said vaccinia virus vector is deficient in D4R synthesis.

87. The method of claim 51, wherein said poxvirus transcriptional regulatory region of said library of polynucleotides functions in the cytoplasm of a poxvirus-infected cell.

88. The method of claim 87 wherein said poxvirus transcriptional regulatory region comprises a promoter.

89. The method of claim 88, wherein said promoter is constitutive.

90. The method of claim 89, wherein said promoter is a vaccinia virus p7.5 promoter.

91. The method of claim 90, wherein said promoter is a synthetic early/late promoter.

92. The method of claim 88, wherein said promoter is a T7 phage promoter active in cells in which T7 RNA polymerase is expressed.

93. The method of claim 87, wherein said poxvirus transcriptional regulatory region comprises a transcriptional termination region.

94. The method of claim 51, wherein said library of polynucleotides is constructed by a method comprising:
(a) Providing a population of host cells permissive for the production of infectious viral particles of said poxvirus vector;
(b) cleaving an isolated linear DNA fragment comprising the genome of said poxvirus vector to produce a first viral fragment and a second viral fragment, wherein said first fragment is nonhomologous with said second fragment;
(c) providing a population of transfer plasmids comprising polynucleotides encoding said plurality of candidate regulator polypeptides through operable association with a transcription control region, wherein each of said polynucleotides is flanked by a 5′ flanking region and a 3′ flanking region, wherein said 5′ flanking region is homologous to said first viral fragment and said 3′ flanking region is homologous to said second viral fragment;
(d) introducing said transfer plasmids and said first and second viral fragments into said population of host cells under conditions wherein each of said transfer plasmids, said first viral fragment, and said second viral fragment undergo in vivo homologous recombination, thereby producing a population of viable modified poxvirus genomes, each comprising a polynucleotide which encodes a candidate regulator polypeptide; and
(e) recovering said population of modified virus genomes.

95. A kit for the identification of a regulator polypeptide, whose expression induces activation of a target transcriptional regulatory region in a eukaryotic host cell, comprising:
(a) a library of polynucleotides encoding, through operable association with a poxvirus transcriptional regulatory region, a plurality of candidate regulator polypeptides, each candidate regulator polypeptide comprising (i) a candidate peptide, and (ii) a molecular scaffold fused to said peptide such that said peptide is displayed on the surface of said candidate regulator polypeptide, wherein said library is constructed in a poxvirus vector; and
(b) a population of eukaryotic host cells capable of expressing said regulator polypeptide, wherein said host cells comprise a target transcriptional regulatory region which is naturally induced in a target cellular process, wherein said target transcriptional regulatory region is operably associated with a polynucleotide encoding a selectable gene product, the expression of which causes said host cells to exhibit a predetermined modified phenotype, and wherein said selectable gene product is expressed upon activation of said target transcriptional regulatory region;
wherein polynucleotides encoding said regulator polypeptides are recoverable from individual host cells selected from the group consisting of: those host cells which exhibit said modified phenotype, and those host cells which fail to exhibit said modified phenotype.

96. An isolated polynucleotide encoding a regulator polypeptide, whose expression influences activation of a target transcriptional regulatory region in a eukaryotic host cell, produced by the method of claim 51.

97. A composition comprising the regulator polypeptide of claim 96, and a pharmaceutically acceptable carrier.


SEQUENCE LISTING (116 sequences)

SEQ ID NO: 1 - protein, 61 aa, Artificial Sequence
  Coiled-coil molecular scaffold
MGCAALESEVSALESEVASLESEVAALGRGDMPLAAVKSKLSAVKSKLASVKSKLAACGPP

SEQ ID NO: 2 - protein, 6 aa, Artificial Sequence
  Loop region of coiled-coil molecular scaffold
GRGDMP

SEQ ID NO: 3 - protein, 69 aa, Artificial Sequence
  Minibody molecular scaffold
MGRNSQATSGFTFSHFYMEWVRGGEYIAASRHKHNKYTTEYSASVKGRYIVSRDTSQSILYLQKKKGPP

SEQ ID NO: 4 - protein, 7 aa, Artificial Sequence
  Nuclear localization signal of SV40 (monkey virus)large T Antigen
PKKKRKV

SEQ ID NO: 5 - protein, 6 aa, Artificial Sequence
  Human retionic acid receptor-beta nuclear localization signal
ARRRRP

SEQ ID NO: 6 - protein, 10 aa, Artificial Sequence
  NFKB p50
EEVQRKRQKL

SEQ ID NO: 7 - protein, 9 aa, Artificial Sequence
  NFKB p65
EEKRKRTYE

SEQ ID NO: 8 - protein, 20 aa, Artificial Sequence
  The Xenopus protein, nucleoplasmin
AVKRPAATKKAGQAKKKKLD

SEQ ID NO: 9 - protein, 31 aa, Artificial Sequence
  Signal sequence from CD8
MASPLTRFLSLNLLLLGESILGSGEAKPQAP

SEQ ID NO: 10 - protein, 21 aa, Artificial Sequence
  Signal sequences from ICAM-2
MSSFGYRTLTVALFTLICCPG

SEQ ID NO: 11 - protein, 51 aa, Artificial Sequence
  Transmembrane domain from CD8, amino acids 145- 195
PQRPEDCRPRGSVKGTGLDFACDIYIWAPLAGICVALLLSLIITLICYHSR

SEQ ID NO: 12 - protein, 33 aa, Artificial Sequence
  Transmembrane domain from ICAM-2, amino acids 224-256
MVIIVTVVSVLLSLFVTSVLLCFIFGQHLRQQR

SEQ ID NO: 13 - protein, 37 aa, Artificial Sequence
  DAF GPI anchor
PNKGSGTTSGTTRLLSGHTCFTLTGLLGTLVTMGLLT

SEQ ID NO: 14 - protein, 14 aa, Artificial Sequence
  Myristylation sequence of c-src
MGSSKSKPKDPSQR

SEQ ID NO: 15 - protein, 26 aa, Artificial Sequence
  Palmitoylation sequence from the G protein- coupled receptor kinase GRK6
LLQRLFSRQDCCGNCSDSEEELPTRL

SEQ ID NO: 16 - protein, 20 aa, Artificial Sequence
  Palmitoylation sequence from rhodopsin
KQFRNCMLTSLCCGKNPLGD

SEQ ID NO: 17 - protein, 19 aa, Artificial Sequence
  Palmitoylation sequence from p21 II-ras 1 protein
LNPPDESGPGCMSCKCVLS

SEQ ID NO: 18 - protein, 5 aa, Artificial Sequence
  Lamp-2 lysosomal degradation sequence
KFERQ

SEQ ID NO: 19 - protein, 36 aa, Artificial Sequence
  Lysosomal membrane sequences from Lamp-I
MLIPIAGFFALAGLVLIVLIAYLIGRKRSHAGYQTI

SEQ ID NO: 20 - protein, 35 aa, Artificial Sequence
  Lysosomal membrane sequences from Lamp-2
LVPIAVGAALAGVLILVLLAYFIGLKHHHAGYEQF

SEQ ID NO: 21 - protein, 27 aa, Artificial Sequence
  Mitochondrial matrix sequence of yeast alcohol dehydrogenase III
MLRTSSLFTRRVQPSLFSRNILRLQST

SEQ ID NO: 22 - protein, 25 aa, Artificial Sequence
  Mitochondrial inner membrane sequence of yeast cytochrome c oxidase subunit IV
MLSLRQSIRFFKPATRTLCSSRYLL

SEQ ID NO: 23 - protein, 63 aa, Artificial Sequence
  Mitochondrial intermembrame space sequence of yeast cytochrome c1
MFSMLSKRWAQRTLSKSFYSTATGAASKSGKLTQKLVTAGVMAGITASTLLYADSLTAEAMTA

SEQ ID NO: 24 - protein, 41 aa, Artificial Sequence
  Mitochondrial outer membrane sequence of yeast 70 kD outer membrane protein
MKSFITRNKTAILATVAATGTAIGAYYYYNQLQQQQQRGKK

SEQ ID NO: 25 - protein, 4 aa, Artificial Sequence
  Endoplasmic reticulum sequence from calreticulin
KDEL

SEQ ID NO: 26 - protein, 15 aa, Artificial Sequence
  Endoplasmic reticulum sequence from adenovirus E3/19K protein
LYLSRRSFIDEKKMP

SEQ ID NO: 27 - protein, 19 aa, Artificial Sequence
  Farnesylation sequence for P21 H-ras 1
LNPPDESGPGCMSCKCVLS

SEQ ID NO: 28 - protein, 15 aa, Artificial Sequence
  Geranylgeranylation sequence for protein rab-5A
LTEPTQPTRNQCCSN

SEQ ID NO: 29 - protein, 9 aa, Artificial Sequence
  Destruction sequences for cyclin Bl
RTALGDIGN

SEQ ID NO: 30 - protein, 20 aa, Artificial Sequence
  Secretory signal sequence from IL-2
MYRMQLLSCIALSLALVTNS

SEQ ID NO: 31 - protein, 29 aa, Artificial Sequence
  Secretory signal sequence from growth hormone
MATGSRTSLLLAFGLLCLPWLQEGSAFPT

SEQ ID NO: 32 - protein, 27 aa, Artificial Sequence
  Secretory signal sequence from preproinsulin
MALWMRLLPLLALLALWGPDPAAAFVN

SEQ ID NO: 33 - protein, 18 aa, Artificial Sequence
  Secretory signal sequence from influenza HA protein
MKAKLLVLLYAFVAGDQI

SEQ ID NO: 34 - protein, 24 aa, Artificial Sequence
  Signal leader sequence from IL4, amino acids 1-24
MGLTSQLLPPLFFLLACAGNFVHG

SEQ ID NO: 35 - protein, 4 aa, Artificial Sequence
  Heterologous signal sequence for the endoplasmic reticulum
KDEL

SEQ ID NO: 36 - protein, 4 aa, Artificial Sequence
  Heterologous signal sequence for the endoplasmic reticulum
DDEL

SEQ ID NO: 37 - protein, 4 aa, Artificial Sequence
  Heterologous signal sequence for the endoplasmic reticulum
DEEL

SEQ ID NO: 38 - protein, 4 aa, Artificial Sequence
  Heterologous signal sequence for the endoplasmic reticulum
QEDL

SEQ ID NO: 39 - protein, 4 aa, Artificial Sequence
  Heterologous signal sequence for the endoplasmic reticulum
RDEL

SEQ ID NO: 40 - protein, 7 aa, Artificial Sequence
  Heterologous signal sequence for the nucleus
PKKKRKV

SEQ ID NO: 41 - protein, 7 aa, Artificial Sequence
  Heterologous signal sequence for the nucleus
PQKKIKS

SEQ ID NO: 42 - protein, 5 aa, Artificial Sequence
  Heterologous signal sequence for the nucleus
QPKKP

SEQ ID NO: 43 - protein, 4 aa, Artificial Sequence
  Heterologous signal sequence for the nucleus
RKKR

SEQ ID NO: 44 - protein, 12 aa, Artificial Sequence
  Heterologous signal sequence for the nucleolar region
RKKRRQRRRAHQ

SEQ ID NO: 45 - protein, 16 aa, Artificial Sequence
  Heterologous signal sequence for the nucleolar region
RQARRNRRRRWRERQR

SEQ ID NO: 46 - protein, 19 aa, Artificial Sequence
  Heterologous signal sequence for the nucleolar region
MPLTRRRPAASQALAPPTP

SEQ ID NO: 47 - protein, 15 aa, Artificial Sequence
  Heterologous signal sequence for the endosomal compartment
MDDQRDLISNNEQLP

SEQ ID NO: 48 - protein, 32 aa, Artificial Sequence
  Heterologous signal sequence for the mitochondrial matrix
MLFNLRXXLNNAAFRHGHNFMVRNFRCGQPLX

SEQ ID NO: 49 - protein, 8 aa, Artificial Sequence
  Myristoylation signal sequence from p56USTRATCK
GCVCSSNP

SEQ ID NO: 50 - protein, 8 aa, Artificial Sequence
  Myristoylation signal sequence from Mul.V gag
GQTVTTPL

SEQ ID NO: 51 - protein, 8 aa, Artificial Sequence
  Myristoylation signal sequence from M-PMV gag
GQELSQHE

SEQ ID NO: 52 - protein, 8 aa, Artificial Sequence
  Myristoylation signal sequence from BLV gag
GNSPSYNP

SEQ ID NO: 53 - protein, 8 aa, Artificial Sequence
  Myristoylation signal sequence from MMTV gag
GVSGSKGQ

SEQ ID NO: 54 - protein, 8 aa, Artificial Sequence
  Myristoylation signal sequence from FCL.V gag
GQTITTPL

SEQ ID NO: 55 - protein, 8 aa, Artificial Sequence
  Myristoylation signal sequence from BaEV gag
GQTLTTPL

SEQ ID NO: 56 - protein, 8 aa, Artificial Sequence
  Myristoylation signal sequence from HTLV-I gag
GQIFSRSA

SEQ ID NO: 57 - protein, 8 aa, Artificial Sequence
  Myristoylation signal sequence from HTLV-II gag
GQIHGLSP

SEQ ID NO: 58 - protein, 8 aa, Artificial Sequence
  Myristoylation signal sequence from HIV (HTLV- III) gag
GARASVLS

SEQ ID NO: 59 - protein, 8 aa, Artificial Sequence
  Myristoylation signal sequence from bovine brain G0 alpha-subunit
GCTLSAEE

SEQ ID NO: 60 - protein, 8 aa, Artificial Sequence
  Myristoylation signal sequence from Hepatitis B Virus pre-S1
GQNLSTSN

SEQ ID NO: 61 - protein, 8 aa, Artificial Sequence
  Myristoylation signal sequence from Polyoma Virus VP2
GAALTILV

SEQ ID NO: 62 - protein, 8 aa, Artificial Sequence
  Myristoylation signal sequence from SV40 Virus VP2
GAALTLLG

SEQ ID NO: 63 - protein, 8 aa, Artificial Sequence
  Myristoylation signal sequence from Poliovirus VP4
GAQVSSQK

SEQ ID NO: 64 - protein, 8 aa, Artificial Sequence
  Myristoylation signal sequence from Bovine Enterovirus VP4
GAQLSRNT

SEQ ID NO: 65 - protein, 8 aa, Artificial Sequence
  Myristoylation signal sequence from cAMP- dependent kinase
GNAAAAKK

SEQ ID NO: 66 - protein, 8 aa, Artificial Sequence
  Myristoylation signal sequence from calcincurin B
GNEASYPL

SEQ ID NO: 67 - protein, 8 aa, Artificial Sequence
  Myristoylation signal sequence from P60SFC
GSSKSKPK

SEQ ID NO: 68 - protein, 6 aa, Artificial Sequence
  Purification sequence that can be used as a peptide rescue sequence
HHHHHH

SEQ ID NO: 69 - protein, 7 aa, Artificial Sequence
  Stability sequence
MGXGGPP

SEQ ID NO: 70 - protein, 2 aa, Artificial Sequence
  Glycine-serine polymers used as a linker
GS

SEQ ID NO: 71 - protein, 5 aa, Artificial Sequence
  Glycine-serine polymers used as a linker
GSGGS

SEQ ID NO: 72 - protein, 4 aa, Artificial Sequence
  Glycine-serine polymers used as a linker
GGGS

SEQ ID NO: 73 - protein, 119 aa, Artificial Sequence
  Intracellular, membrane-anchored, linked (i.e. tethered) free peptide
MRPLAGGEHTMASPLTRFLSLNLLLLGESIILGSGPQRPEDCRPRGSVKGTGLDFACDIYIWAPLAGICVALLLSLIITLICYHSRGSGGSGSGGSGSGGSGSGGSGSGGSGGGXGGPP

SEQ ID NO: 74 - protein, 168 aa, Artificial Sequence
  Intracellular, membrane-anchored, linked coiled coil
MRPLAGGEHTMASPLTRFLSLNLLLLGESIILGSGPQRPEDCRPRGSVKGTGLDFACDIYIWAPLAGICVALLLSLIITLICYHSRGSGGSGSGGSGSGGSGSGGSGSGGSGGGCAALESEVSALESEVASLESEVAALXLAAVKSKLSAVKSKLASVKSKLAACGPP

SEQ ID NO: 75 - protein, 121 aa, Artificial Sequence
  Surface-tethered extracellular, non-constrained
MRPLAGGEHTMASPLTRFLSLNLLLLGESHLGSGGGXGGSGGSGSGGSGSGGSGSGGSGSGGSGGGPQRPEDCRPRGSVKGTGLDFACDIYIWAPLAGICVALLLSLIITLICYHSRGGPP

SEQ ID NO: 76 - protein, 171 aa, Artificial sequence
  Surface-tethered, extracellular constrained
MRPLAGGEHTMASPLTRFLSLNLLLLGESIILGSGGGCAALESEVSALESEVASLESEVAALXLAAVKSKLSAVKSKLASVKSKLAACGGSGGSGSGGSGSGGSGSGGSGSGGSGGGPQRPEDCRPRGSVKGTGLDFACDIYIWAPLAGICVALLLSLHTLICYHSRGGPP

SEQ ID NO: 77 - protein, 42 aa, Artificial Sequence
  Secreted, non-constrained
MRPLAGGEHTMASPLTRFLSLNLLLLGESIILGSGGGXGGPP

SEQ ID NO: 78 - protein, 90 aa, Artificial Sequence
  Secreted, constrained
MRPLAGGEHTMASPLTRFLSLNLLLLGESIILGSGGGAALESEVSALESEVASLESEVAALXLAAVKSKLSAVKSKLASVKSKLAACGPP

SEQ ID NO: 79 - protein, 9 aa, Artificial Sequence
  Oligos/peptides wherein Xaa is a randomized amino acid residue
XXXPPXPXX

SEQ ID NO: 80 - DNA, 63 bp, Artificial Sequence
  Oligos/peptides
atgggcnnknnknnknnknnkagacctctgcctccasbkgggsbksbkggaggcccaccttaa

SEQ ID NO: 81 - protein, 20 aa, Artificial Sequence
  Oligos/peptides
MGXXXXXRPLPPXPXXGGPP

SEQ ID NO: 82 - protein, 12 aa, Artificial Sequence
  Cyclized peptide library
CXXXXXXXXXXC

SEQ ID NO: 83 - DNA, 21 bp, Artificial Sequence
  Gus sense specific primer
atgttacgtcctgtagaaacc

SEQ ID NO: 84 - DNA, 21 bp, Artificial Sequence
  Gus antisense specific primer
tcattgtttgcctccctgctg

SEQ ID NO: 85 - DNA, 28 bp, Artificial Sequence
  NX-Gus Sense primer
aaagcggccgccccgggatgttacgtcc

SEQ ID NO: 86 - DNA, 29 bp, Artificial Sequence
  AA-Gus antisense primer
aaagggcccggcgcgcctcattgtttgcc

SEQ ID NO: 87 - DNA, 37 bp, Artificial Sequence
  D4R-Sense primer
aaaggatccataatgaattcagtgactgtatcacacg

SEQ ID NO: 88 - DNA, 34 bp, Artificial Sequence
  D4R Antisense primer
cttgcggccgcttaataaataaacccttgagccc

SEQ ID NO: 89 - DNA, 34 bp, Artificial Sequence
  D4R Flank sense primer
attgagctcttaatacttttgtcgggtaacagag

SEQ ID NO: 90 - DNA, 29 bp, Artificial Sequence
  D4R Flank antisense primer
ttactcgagagtgtcgcaatttggatttt

SEQ ID NO: 91 - DNA, 29 bp, Artificial Sequence
  7.5 Gus Sense primer
aaagaattcctttattgtcatcggccaaa

SEQ ID NO: 92 - DNA, 30 bp, Artificial Sequence
  7.5 Gus antisense primer
aatctgcagtcattgtttgcctccctgctg

SEQ ID NO: 93 - DNA, 37 bp, Artificial Sequence
  D4R-sense primer
aaagaattcataatgaattcagtgactgtatcacacg

SEQ ID NO: 94 - DNA, 32 bp, Artificial Sequence
  D4R-antisense primer
cttggatccttaataaataaacccttgagccc

SEQ ID NO: 95 - DNA, 29 bp, Artificial Sequence
  D4R left flank sense primer
aataagctttgactccagatacatatgga

SEQ ID NO: 96 - DNA, 25 bp, Artificial Sequence
  D4R left flank antisense primer
aatctgcagcaccagttccatcttt

SEQ ID NO: 97 - DNA, 23 bp, Artificial Sequence
  D4R right flank sense primer
aatggatcctcatccagcggcta

SEQ ID NO: 98 - DNA, 27 bp, Artificial Sequence
  D4R right flank antisense primer
aatgagctctagtacctacaacccgaa

SEQ ID NO: 99 - DNA, 28 bp, Artificial Sequence
  EL-Gus sense primer
aaagtcgacggccaaaaattgaaatttt

SEQ ID NO: 100 - DNA, 25 bp, Artificial Sequence
  EL-Gus antisense primer
aatggatcctcattgtttgcctccc

SEQ ID NO: 101 - DNA, 62 bp, Artificial Sequence
  Single stranded primer, +37BC Sense”, encoding the BC Loop region of FN3
aaaatgatcagctgggatgctcctgcannknnkgtgcgttattaccgtatcacgtacggtga

SEQ ID NO: 102 - DNA, 18 bp, Artificial Sequence
  BC antisense primer
tcaccgtacgtgatacgg

SEQ ID NO: 103 - DNA, 70 bp, Artificial Sequence
  A single stranded primer, “FG Sense”, encoding these three randomized codons
gggtgtcgactataccatcactgtatacgctgttactggcnnknnknnkagcccagcgagctccaagcca

SEQ ID NO: 104 - DNA, 18 bp, Artificial Sequence
  FG Antisense primer
tggcttggagctcgctgg

SEQ ID NO: 105 - DNA, 15 bp, Artificial Sequence
  BglII-NcoI Sense primer
gatctcggtaaccgc

SEQ ID NO: 106 - DNA, 15 bp, Artificial Sequence
  BglII-NcoI Antisense primer
catggcggttaccga

SEQ ID NO: 107 - DNA, 15 bp, Artificial Sequence
  XhoI-XmaIII sense primer
ggccgaaataaccgc

SEQ ID NO: 108 - DNA, 15 bp, Artificial Sequence
  XhoI-XmaIII antisense primer
tcgagcggttatttc

SEQ ID NO: 109 - DNA, 15 bp, Artificial Sequence
  H3-NcoI sense primer
agcttcggtaaccgc

SEQ ID NO: 110 - DNA, 15 bp, Artificial Sequence
  H3-NcoI antisense primer
catggcggttaccga

SEQ ID NO: 111 - DNA, 20 bp, Artificial Sequence
  PstI-XmaIII sense primer
ggccggaaataaccgctgca

SEQ ID NO: 112 - DNA, 12 bp, Artificial Sequence
  PstI-XmaIII antisense primer
gcggttatttcc

SEQ ID NO: 113 - DNA, 16 bp, Artificial Sequence
  SalI-NcoI sense primer
ctgaggaaataaccgc

SEQ ID NO: 114 - DNA, 16 bp, Artificial Sequence
  SalI-NcoI antisense primer
catggcggttatttcc

SEQ ID NO: 115 - protein, 9 aa, Artificial Sequence
  H-2Kd restricted CTL epitope
GYKAGMIHI

SEQ ID NO: 116 - protein, 8 aa, Artificial Sequence
  Epitope  known to bind with high affinity to the mouse class I MHC molecule Kb
SIINFEKL